SEQUENCE LISTING (242 sequences)

SEQ ID NO: 1 - protein, 60 aa, Actinidia eriantha
TAIFGVSRALVHVRSVRRKGCVVESNPSPHGGRGALPSEGGSPSDLLFLAGGGHFAFSVY

SEQ ID NO: 2 - protein, 62 aa, Cucumis sativus
TAIHVVSRSFFHVRAVRRKGCITPTNPSPHGGRGALPSEGGSPSDLLFLAGGGFSCFFSSSY

SEQ ID NO: 3 - protein, 58 aa, Glycine max
TAILRVSRSLIHVPTVRRRTGCVTATNPSPHGGRGALPSEGGSPSDLLFLAGGGSAVF

SEQ ID NO: 4 - protein, 61 aa, Solanum lycopersicum
TAIHKVNRRPLLHVPAVRRKGCVTATNPAPHGGRGALPSEGGSPSDLLFLAGGGSFLSFSY

SEQ ID NO: 5 - protein, 59 aa, Vitis vinifera
TAIQRIPPPLIHVRAVRRKGCVIESNPSPHGGRGALPSEGGSPSDLLFLAGGGSNAFLC

SEQ ID NO: 6 - protein, 64 aa, Arabidopsis thaliana
TAIHGISRGVSSHVHIVRQKGCLIETNPLPHGGRGALPSEGGSPSDLLFLAGGGSSFNFFSFRF

SEQ ID NO: 7 - protein, 57 aa, Arabidopsis thaliana
TAIYGIKPRPLSFHVQRKGCLIITNPLPHGGRGALPSEGGSPSDLLFLAGGGSTNSH

SEQ ID NO: 8 - protein, 64 aa, Malus domestica
TAIPRAPRPLVHVRGVGRKGCVIESNPSPHGGRGALPSEGGSPSDLLFLAGGGSPSSVFLFCFY

SEQ ID NO: 9 - protein, 61 aa, Medicago truncatula
TAFLRVYRSLSHARTVRRKGCCLTPTNPSPHGGRGALPSEGGSPSDLLFLAGGGVFAPSSF

SEQ ID NO: 10 - protein, 63 aa, Populus trichocarpa
TAIHGVTRSLIHVRAVRRKGCVIESSNPSPHGGRGALPSEGGSPSDLLFLAGGGFFFFCLVVV

SEQ ID NO: 11 - protein, 80 aa, Picea sitchensis
TAIKRILRFQPHDRRRILRSRNAGCALESTPSPHGGRGALPSEGGSPSDLLFLAGGGCDRALFCLAHPFLLSRGEIIWER

SEQ ID NO: 12 - protein, 57 aa, Physcomitrella patens
MSKDFYRAGIQLSRSLSSSLSLHGGRGAAPSEGGRPSDLSALAGGGFLSNFHHGADP

SEQ ID NO: 13 - protein, 60 aa, Actinidia arguta
TAILGVSRPLIHVRSVRRKGCVVESNPSPHGGRGALPSEGGSPSDLLFLAGGGHFAFSVY

SEQ ID NO: 14 - protein, 60 aa, Actinidia eriantha; identical to SEQ ID NO: 13

SEQ ID NO: 15 - protein, 60 aa, Actinidia chinensis; identical to SEQ ID NO: 13

SEQ ID NO: 16 - protein, 64 aa, Fragaria vulgaris
TAIHRVSRPLIHVRRTVRRKGCVIESNPSPHGGRGALPSEGGSPSDLLFLAGGGVHVSDLRFFF

SEQ ID NO: 17 - protein, 61 aa, Solanum tuberosum; identical to SEQ ID NO: 4

SEQ ID NO: 18 - protein, 55 aa, Chlamydomonas reinhardtii
MHLREPVKTAFSEAARVQSAASQPATANRCSGGRGAAPSCGGKPKDALSKAGGGQ

SEQ ID NO: 19 - protein, 60 aa, Zea mays
  SITE 56: note = misc_feature - Xaa can be any naturally occurring amino acid
TASVAAPRRGPAAAQVEPTGTIASAVASSPAPHGGRGALPSAGGSPSDLLFLAGGXAARL

SEQ ID NO: 20 - protein, 61 aa, Selaginella moellendorffii
TASIELLSRHPIICYGHSVSSCKTLLSSLSCHGGRGASPSEGGHPSDLTFLAGGGLLLGAP

SEQ ID NO: 21 - protein, 28 aa, Actinidia eriantha
NPSPHGGRGALPSEGGSPSDLLFLAGGG

SEQ ID NO: 22 - protein, 28 aa, Cucumis sativus
NPSPHGGRGALPSEGGSPSDLLFLAGGG

SEQ ID NO: 23 - protein, 28 aa, Glycine max
NPSPHGGRGALPSEGGSPSDLLFLAGGG

SEQ ID NO: 24 - protein, 28 aa, Solanum lycopersicum
NPAPHGGRGALPSEGGSPSDLLFLAGGG

SEQ ID NO: 25 - protein, 28 aa, Vitis vinifera
NPSPHGGRGALPSEGGSPSDLLFLAGGG

SEQ ID NO: 26 - protein, 28 aa, Arabidopsis thaliana
NPLPHGGRGALPSEGGSPSDLLFLAGGG

SEQ ID NO: 27 - protein, 28 aa, Arabidopsis thaliana
NPLPHGGRGALPSEGGSPSDLLFLAGGG

SEQ ID NO: 28 - protein, 28 aa, Malus domestica
NPSPHGGRGALPSEGGSPSDLLFLAGGG

SEQ ID NO: 29 - protein, 28 aa, Medicago truncatula
NPSPHGGRGALPSEGGSPSDLLFLAGGG

SEQ ID NO: 30 - protein, 28 aa, Populus trichocarpa
NPSPHGGRGALPSEGGSPSDLLFLAGGG

SEQ ID NO: 31 - protein, 28 aa, Picea sitchensis
TPSPHGGRGALPSEGGSPSDLLFLAGGG

SEQ ID NO: 32 - protein, 28 aa, Physcomitrella patens
SLSLHGGRGAAPSEGGRPSDLSALAGGG

SEQ ID NO: 33 - protein, 28 aa, Actinidia arguta
NPSPHGGRGALPSEGGSPSDLLFLAGGG

SEQ ID NO: 34 - protein, 28 aa, Actinidia eriantha
NPSPHGGRGALPSEGGSPSDLLFLAGGG

SEQ ID NO: 35 - protein, 28 aa, Actinidia chinensis
NPSPHGGRGALPSEGGSPSDLLFLAGGG

SEQ ID NO: 36 - protein, 28 aa, Fragaria vulgaris
NPSPHGGRGALPSEGGSPSDLLFLAGGG

SEQ ID NO: 37 - protein, 28 aa, Solanum tuberosum
NPAPHGGRGALPSEGGSPSDLLFLAGGG

SEQ ID NO: 38 - protein, 28 aa, Chlamydomonas reinhardtii
ANRCSGGRGAAPSCGGKPKDALSKAGGG

SEQ ID NO: 39 - protein, 26 aa, Zea mays
PAPHGGRGALPSAGGSPSDLLFLAGG

SEQ ID NO: 40 - protein, 28 aa, Selaginella moellendorffii
SLSCHGGRGASPSEGGHPSDLTFLAGGG

SEQ ID NO: 41 - DNA, 180 bp, Actinidia eriantha
  source 1..180: mol_type = genomic DNA
acggctatactcggagtttctcggccgctcatacatgtccggtctgtacgacgcaagggttgtgtagtcgagagcaacccttcgccgcacggaggacgtggcgccttgccgtccgaaggcggtagcccctccgacctcctcttcctcgccggcggcggtcacttcgctttctccgtctac

SEQ ID NO: 42 - DNA, 186 bp, Cucumis sativus
  source 1..186: mol_type = genomic DNA
acggctatacacgtagtttcccggtcgttctttcatgtcagggctgtacgacggaagggttgtataactccgacaaacccttcgccgcacggcggacgtggtgctttgccttccgaaggtggtagtccttctgatcttctttttctcgccggcggtggtttctcttgcttcttctcttcttcgtat

SEQ ID NO: 43 - DNA, 174 bp, Glycine max
  source 1..174: mol_type = genomic DNA
acggctattctaagagtgtctcgctctctcattcatgtcccaactgtacgacggaggacaggttgcgtaactgccaccaacccttcgccgcacggtgggcgtggtgctttgccttctgaaggtggtagcccttcagacttgctcttcttagctggtggtggttctgctgtcttc

SEQ ID NO: 44 - DNA, 183 bp, Solanum lycopersicum
  source 1..183: mol_type = genomic DNA
acggctatacacaaagtaaaccgccgaccacttttacatgttccagcagtacgtcgtaagggttgtgtaacagctactaaccctgcgccgcacggtggacgtggcgctttgccttctgaaggtggtagtccttccgacctcctcttccttgccggcggcggttctttcctctccttctcctac

SEQ ID NO: 45 - DNA, 177 bp, Vitis vinifera
  source 1..177: mol_type = genomic DNA
acggctatacaaagaattccgccgcctctcatacatgtccgggcggtacgacgcaagggytgtgtaattgagagcaacccttcgccgcacggcgggcgtggcgctttgccttcagaaggcggtagtccctctgatctgctcttcctcgccggcggtggttccaacgctttcctctgc

SEQ ID NO: 46 - DNA, 192 bp, Arabidopsis thaliana
  source 1..192: mol_type = genomic DNA
acggctatacacgggatatcacggggtgttagctcacatgtccatattgtccgacagaagggttgtttaatcgaaactaatcctttgccgcacggaggacgtggagctctgccgtctgaaggcggcagcccttccgatctcctctttctcgccggtggcggttccagctttaacttcttttcctttaggttt

SEQ ID NO: 47 - DNA, 183 bp, Arabidopsis thaliana
  source 1..183: mol_type = genomic DNA
acggcttttcttagagtttatcggtcactttcacatgcccgaactgtacgacgtaagggttgttgcttaactccgactaacccttcgccgcacggtgggcgtggtgctctgccttctgaaggtggtagcccctccgatcttctcttcctcgccggtggtggtgtctttgctccttcttccttc

SEQ ID NO: 48 - DNA, 192 bp, Malus domestica
  source 1..192: mol_type = genomic DNA
acggctatacccagagctcctcggccgctcgttcatgtccggggtgtcggacgaaagggttgtgtaattgagagcaacccttcgccgcacggcgggcgtggtgctttgccttccgaaggcggtagcccgtctgacctcctcttcctcgccggtggcggctccccttcctctgtttttctcttctgcttttat

SEQ ID NO: 49 - DNA, 183 bp, Medicago truncatula; identical to SEQ ID NO: 47
  source 1..183: mol_type = genomic DNA

SEQ ID NO: 50 - DNA, 189 bp, Populus trichocarpa
  source 1..189: mol_type = genomic DNA
acggctatacacggagtaactcggtccctaattcatgtccgggctgttcgacgtaagggctgtgtaatagagagcagcaacccttcgccgcacggtggacgtggtgctttaccctcggaaggcggtagcccttctgatctcctctttctagctggtggcggtttcttcttcttctgtttagtagtagtt

SEQ ID NO: 51 - DNA, 240 bp, Picea sitchensis
  source 1..240: mol_type = genomic DNA
acggctattaaacgtattttgcggttccagccccatgatcgcagaagaattctgcggagcaggaacgctggttgtgcactagagagtaccccgtcgccgcacggtgggagaggagccttgccctcggaaggcgggagcccctctgatctcctctttctcgcaggaggcggttgcgaccgagcccttttctgcctcgcccatccatttcttctgagcagaggagaaataatttgggagagg

SEQ ID NO: 52 - DNA, 171 bp, Physcomitrella patens
  source 1..171: mol_type = genomic DNA
atgagcaaagacttctaccgtgcaggaatccaactgtcccgatctctctcttcctctctttccttgcacggtggtcggggggccgcccccagtgaaggcggtcgtccctctgacctatctgcacttgctgggggaggttttctctcaaactttcaccacggtgctgatcct

SEQ ID NO: 53 - DNA, 180 bp, Actinidia arguta
  source 1..180: mol_type = genomic DNA
acggctatactcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggtggcggtcacttcgctttctccgtctac

SEQ ID NO: 54 - DNA, 180 bp, Actinidia eriantha; identical to SEQ ID NO: 41
  source 1..180: mol_type = genomic DNA

SEQ ID NO: 55 - DNA, 180 bp, Actinidia chinensis
  source 1..180: mol_type = genomic DNA
acggctatactcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctac

SEQ ID NO: 56 - DNA, 192 bp, Fragaria vulgaris
  source 1..192: mol_type = genomic DNA
acggctatacacagagtttcacggccactcattcatgtccgccggactgtccgacgtaagggttgtgtaattgagagcaacccttcgccgcacggcgggcgtggtgctttgccgtccgaaggaggaagtccttccgacctccttttcctcgccggcggtggtgtccacgtttccgacttgcgtttctttttc

SEQ ID NO: 57 - DNA, 183 bp, Solanum tuberosum
  source 1..183: mol_type = genomic DNA
acggctatacacaaagtaaaccgccgaccacttttacatgttccagcagtacgtcgtaagggttgtgtaacagctactaaccctgcgccgcacggtggacgtggcgctttgccctctgaaggtggcagtccttccgacctcctcttccttgccggcggcggttctttcctctccttctcctac

SEQ ID NO: 58 - DNA, 165 bp, Chlamydomonas reinhardtii
  source 1..165: mol_type = genomic DNA
atgcatttgcgtgagccggtaaaaacggcgttctctgaggcagcccgcgttcagtcggctgcgtcgcagcctgcgaccgccaatcggtgctccggtggccgtggtgcagccccctcgtgcggtggcaagcccaaggacgctctgagcaaggcgggcggtggccag

SEQ ID NO: 59 - DNA, 174 bp, Zea mays
  source 1..174: mol_type = genomic DNA
acggctagcgttgcagccccccggcgcggccccgcagctgcgcaggtggagcccacgggcaccatcgcctccgctgtcgcgtccagccccgctccgcacggcggccgcggggcgctgccctcggccggaggaagcccgtccgatctcctcttcctcgccggcggsgktcgcctc

SEQ ID NO: 60 - DNA, 183 bp, Selaginella moellendorffii
  source 1..183: mol_type = genomic DNA
acggcttcaattgagcttttgagtcgacaccccatcatttgttatggacacagtgtttccagttgtaagactctgctgagttccctctcttgccacggtggtcgaggggctagcccctcggaaggaggacacccttcagatcttacctttcttgctggtggtggccttcttctcggagcacca

SEQ ID NO: 61 - DNA, 105 bp, Actinidia eriantha
  source 1..105: mol_type = genomic DNA
aagggttgtgtagtcgagagcaacccttcgccgcacggaggacgtggcgccttgccgtccgaaggcggtagcccctccgacctcctcttcctcgccggcggcggt

SEQ ID NO: 62 - DNA, 105 bp, Cucumis sativus
  source 1..105: mol_type = genomic DNA
aagggttgtataactccgacaaacccttcgccgcacggcggacgtggtgctttgccttccgaaggtggtagtccttctgatcttctttttctcgccggcggtggt

SEQ ID NO: 63 - DNA, 108 bp, Glycine max
  source 1..108: mol_type = genomic DNA
aggacaggttgcgtaactgccaccaacccttcgccgcacggtgggcgtggtgctttgccttctgaaggtggtagcccttcagacttgctcttcttagctggtggtggt

SEQ ID NO: 64 - DNA, 105 bp, Solanum lycopersicum
  source 1..105: mol_type = genomic DNA
aagggttgtgtaacagctactaaccctgcgccgcacggtggacgtggcgctttgccttctgaaggtggtagtccttccgacctcctcttccttgccggcggcggt

SEQ ID NO: 65 - DNA, 105 bp, Vitis vinifera
  source 1..105: mol_type = genomic DNA
aagggytgtgtaattgagagcaacccttcgccgcacggcgggcgtggcgctttgccttcagaaggcggtagtccctctgatctgctcttcctcgccggcggtggt

SEQ ID NO: 66 - DNA, 105 bp, Arabidopsis thaliana
  source 1..105: mol_type = genomic DNA
aagggttgtttaatcgaaactaatcctttgccgcacggaggacgtggagctctgccgtctgaaggcggcagcccttccgatctcctctttctcgccggtggcggt

SEQ ID NO: 67 - DNA, 105 bp, Arabidopsis thaliana
  source 1..105: mol_type = genomic DNA
aagggttgtttaatcataactaatcctttgcctcacggaggacgtggagctctgccgtctgaaggcggcagtccctccgatctcctcttcctcgccggaggcggt

SEQ ID NO: 68 - DNA, 105 bp, Malus domestica
  source 1..105: mol_type = genomic DNA
aagggttgtgtaattgagagcaacccttcgccgcacggcgggcgtggtgctttgccttccgaaggcggtagcccgtctgacctcctcttcctcgccggtggcggc

SEQ ID NO: 69 - DNA, 107 bp, Medicago truncatula
  source 1..107: mol_type = genomic DNA
aagggttgttgcttaactccgactaacccttcgccgcacggtgggcgtggtgctctgccttctgaaggtggtagcccctccgatcttctcttcctcgccggtggtgg

SEQ ID NO: 70 - DNA, 108 bp, Populus trichocarpa
  source 1..108: mol_type = genomic DNA
aagggctgtgtaatagagagcagcaacccttcgccgcacggtggacgtggtgctttaccctcggaaggcggtagcccttctgatctcctctttctagctggtggcggt

SEQ ID NO: 71 - DNA, 102 bp, Picea sitchensis
  source 1..102: mol_type = genomic DNA
ggttgtgcactagagagtaccccgtcgccgcacggtgggagaggagccttgccctcggaaggcgggagcccctctgatctcctctttctcgcaggaggcggt

SEQ ID NO: 72 - DNA, 78 bp, Physcomitrella patens
  source 1..78: mol_type = genomic DNA
tccttgcacggtggtcggggggccgcccccagtgaaggcggtcgtccctctgacctatctgcacttgctgggggaggt

SEQ ID NO: 73 - DNA, 105 bp, Actinidia arguta
  source 1..105: mol_type = genomic DNA
aagggttgtgtagtcgagagcaacccttcgccgcacggcggacgtggcgccttgccgtccgaaggcggtagcccctccgacctcctcttcctcgccggtggcggt

SEQ ID NO: 74 - DNA, 105 bp, Actinidia eriantha; identical to SEQ ID NO: 61
  source 1..105: mol_type = genomic DNA

SEQ ID NO: 75 - DNA, 105 bp, Actinidia chinensis
  source 1..105: mol_type = genomic DNA
aagggttgtgtagtcgagagcaacccttcgccgcacggcggacgtggcgccttgccgtccgaaggcggtagcccctccgacctcctcttcctcgccggcggcggt

SEQ ID NO: 76 - DNA, 105 bp, Fragaria vulgaris
  source 1..105: mol_type = genomic DNA
aagggttgtgtaattgagagcaacccttcgccgcacggcgggcgtggtgctttgccgtccgaaggaggaagtccttccgacctccttttcctcgccggcggtggt

SEQ ID NO: 77 - DNA, 105 bp, Solanum tuberosum
  source 1..105: mol_type = genomic DNA
aagggttgtgtaacagctactaaccctgcgccgcacggtggacgtggcgctttgccctctgaaggtggcagtccttccgacctcctcttccttgccggcggcggt

SEQ ID NO: 78 - DNA, 69 bp, Chlamydomonas reinhardtii
  source 1..69: mol_type = genomic DNA
ggtggccgtggtgcagccccctcgtgcggtggcaagcccaaggacgctctgagcaaggcgggcggtggc

SEQ ID NO: 79 - DNA, 81 bp, Zea mays
  source 1..81: mol_type = genomic DNA
cccgctccgcacggcggccgcggggcgctgccctcggccggaggaagcccgtccgatctcctcttcctcgccggcggsgkt

SEQ ID NO: 80 - DNA, 72 bp, Selaginella moellendorffii
  source 1..72: mol_type = genomic DNA
cacggtggtcgaggggctagcccctcggaaggaggacacccttcagatcttacctttcttgctggtggtggc

SEQ ID NO: 81 - DNA, 655 bp, Actinidia eriantha
  misc_feature 219: note = n is a, c, g, or t
  misc_feature 356: note = n is a, c, g, or t
  misc_feature 364: note = n is a, c, g, or t
  source 1..655: mol_type = genomic DNA
tctctccgtccctctcaatagttgtctccattcgcagtaaaatcactaaggccgctcgtccctcagtgcacaccacggcccctccacagccgcattcacctctctctctctctctctgctctatctatatatccccccaatctggcctctcttcacctcacccccaaaatctacacaaaatcaatccttcatcttccacatcggcctccaaaacccacnctcttctccacaatccagacacaccttgagcggctggcgttgagcgaatagatagagatagagagagagattttctgcttcgatcgggggtaaaacccggtgtttgacaagttgtagacatcacggctatactcggnagtttctncggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctactagcttattaggtttattcttacttagtgagtaattcgtcctattatagttcgtaagttcatcaaagatctgttacttgattcgtctttcgttgctcgagtcttggtgttttttgcgttttctgagttcgag

SEQ ID NO: 82 - DNA, 600 bp, Cucumis sativus
  source 1..600: mol_type = genomic DNA
tccctttctatataattgcttcattccccacctttccatgttcgtgcagcccattcaatcccctcattttaacccacttcctctttttctttttctccttcctcttccagttcccttttccccatctgggttctcttgatttctcttaaaatccaacaacccatgttcgactttgaggaattggtgcgttggaattgagttttcggagaagatttttcgttttttatcacaacccatctactccaggtaaggggtaaaacccgggttcgtcaggctgtagacatcacggctatacacgtagtttcccggtcgttctttcatgtcagggctgtacgacggaagggttgtataactccgacaaacccttcgccgcacggcggacgtggtgctttgccttccgaaggtggtagtccttctgatcttctttttctcgccggcggtggtttctcttgcttcttctcttcttcgtattagctttccgtttgtgttttagctctaccggtttaggatttgacatcagcaagtttctgtttcgcgtttatttcttttgggtgggggagattttggtgttcggtttggtttgaattagaagcagacgat

SEQ ID NO: 83 - DNA, 429 bp, Glycine max
  source 1..429: mol_type = genomic DNA
gacaaactcacaccacatccatctttgccttctgcatgttggttctctgtaaacagatactgcaaaagaagaataatattgaatatttggttgggttgaatccctgggttgaagcgttgcagacatcacggctattctaagagtgtctcgctctctcattcatgtcccaactgtacgacggaggacaggttgcgtaactgccaccaacccttcgccgcacggtgggcgtggtgctttgccttctgaaggtggtagcccttcagacttgctcttcttagctggtggtggttctgctgtcttctagcttcttcttaactcttttttctttttactacttttaagctaccttgttttcaaaataacaaaaacaaacactttcttacttcataagatcaccttttcttcatcttctacttctccttcactaaa

SEQ ID NO: 84 - DNA, 680 bp, Solanum lycopersicum
  source 1..680: mol_type = genomic DNA
atttgttcggtatactgtaaccccctgtttgcgattggccttgtagccccgttttacatcttccagagactccatttgtatcggttcacatacagtagcaaagcgccattatcttactctaccccattggcaaacccacagccacaattttccaatcctccattatcccttctacaattttctatataaatacccacatctctctgctctactcccttattatcaacaacaaccaccaaatttcttcttttttttcttcgatagtagcaatctatcaacaaaaacagagaccccatcacaagaatcttggaattttagtgttgggtttaagaggaaaaggggttattgtattttgcagttttgagggtaaagcccagtttaacaagttgtagacatcacggctatacacaaagtaaaccgccgaccacttttacatgttccagcagtacgtcgtaagggttgtgtaacagctactaaccctgcgccgcacggtggacgtggcgctttgccttctgaaggtggtagtccttccgacctcctcttccttgccggcggcggttctttcctctccttctcctactagatatagttatacttactatagatctctagcttattacgtacagttgtatctagtattctattgattattcgaagaaaacacacaaaaagaagtaaag

SEQ ID NO: 85 - DNA, 600 bp, Vitis vinifera
  source 1..600: mol_type = genomic DNA
acacaaaagcctctcaagtccaacagataggccgccttcttcctcatttcactgcagctagcttcctatttttcttcttccaaaatccagggatttcaagagaagagaaagagaaataagggttctgcgggttgtgggtcggtgtgttttgtggttttctatatagggtttctaaggagggagggattttgatttgggttgtgacggtggtggggtaaaacccttgttcgacgagttgtagacatcacggctatacaaagaattccgccgcctctcatacatgtccgggcggtacgacgcaagggytgtgtaattgagagcaacccttcgccgcacggcgggcgtggcgctttgccttcagaaggcggtagtccctctgatctgctcttcctcgccggcggtggttccaacgctttcctctgctagtttaggcttatattctgcataatatagctactgtctttaggattagatcaaccaatccgtatcgaacactcgatctctcgctttagccatttctttagatcaatcaatccgtatcgaacaatcgatctctcgctttagccatttctttaggttggtgtttgagttcttgaaaaa

SEQ ID NO: 86 - DNA, 503 bp, Arabidopsis thaliana
  source 1..503: mol_type = genomic DNA
gtatcatcaaaaaacacctcaaagaattattcattcaggcatcttctcaaatttttgtttgtgaaaaaaacccacatcaaaagatctctcatttattcgtttcgtttctgctgttttgagtgtcgggttcgttttagctgtaatctttttttccggcgttcgatttgaaaaaatccggggaacaggtgatcggaatcacggctatacacgggatatcacggggtgttagctcacatgtccatattgtccgacagaagggttgtttaatcgaaactaatcctttgccgcacggaggacgtggagctctgccgtctgaaggcggcagcccttccgatctcctctttctcgccggtggcggttccagctttaacttcttttcctttaggttttaggagttagggtttgttagtgttttttccttcttctttttttggtgctcttgaatcgcttttttcttgggggaagttttttcttttgctcttcgaaatttgtcttttttgaga

SEQ ID NO: 87 - DNA, 420 bp, Arabidopsis thaliana
  source 1..420: mol_type = genomic DNA
caaaatctcacacatcaaacacgtgaatttgctctctttctggttcactcacctaccattactataagtctgaaagagtgattgaaacccacctcgaaaaatctatccttttttttgttttccttctccggcgaatccccggggagattggtaatcggtaatcacggctatttacgggataaagccacggcctttgagctttcatgtccaacgaaagggttgtttaatcataactaatcctttgcctcacggaggacgtggagctctgccgtctgaaggcggcagtccctccgatctcctcttcctcgccggaggcggttccacaaatagccactaaccctaaccctttttctaattaggtttttagttcttagagtcctgtattaatctgttatttcgagattataatatttgtgagca

SEQ ID NO: 88 - DNA, 554 bp, Malus domestica
  source 1..554: mol_type = genomic DNA
ccccacctgctcccccaattccccagtacccatttgtgtttttcgattcagttcgaaaccaggcggtcttcacctttctggttgttttcctatctcggttttaaggaggaagaagaaaggaaggcgttttgatcattttcttttcgaatttcttttggggtaagacccaggttcgacgagttgtagacatcacggctatacccagagctcctcggccgctcgttcatgtccggggtgtcggacgaaagggttgtgtaattgagagcaacccttcgccgcacggcgggcgtggtgctttgccttccgaaggcggtagcccgtctgacctcctcttcctcgccggtggcggctccccttcctctgtttttctcttctgcttttattagttttatttttatagagtttcttgcttagatttttagagattttttgttctataaagcgctcgagtagatcgtatttttgttttcgggggttttttttttttgtggtgtttgatttttactgagaaatcgagaaaaaaagggagagagagagagagaaagaaggcgagt

SEQ ID NO: 89 - DNA, 600 bp, Medicago truncatula
  source 1..600: mol_type = genomic DNA
aacaaaacaccctcctcgtgcttgattctagtacaagtataccaaactcattcacaaccacatttccttaatctcttttcattttctcttatctatataacactttacaatctccacctcattttcttcatcaaaaacaattcatctttcttctttgctatcatcactaaattttcttgagaattcagaaaataaaagagagatttgaatgtttgggttgattccctgtattgacacgttgtagacatcacggcttttcttagagtttatcggtcactttcacatgcccgaactgtacgacgtaagggttgttgcttaactccgactaacccttcgccgcacggtgggcgtggtgctctgccttctgaaggtggtagcccctccgatcttctcttcctcgccggtggtggtgtctttgctccttcttccttctaatttcttgtttttagtttaactttctttagatttttacgactcaaaactataagctaaaactagactttgtaagtaagatttatttgaaggtgcttcttcttcttcttctttcttcttcttgttgttaaaaaaaaaagtatttttgtgtttggtttggtgaaaatg

SEQ ID NO: 90 - DNA, 621 bp, Populus trichocarpa
  source 1..621: mol_type = genomic DNA
gaaccattagtgtgtaccctcctcaccaaaaaagaataaaaactctcacaaccaaaccccaattcacagcaaaaccataaccacaattcccacttctctcttatctatacaatatcccatctcctttttcttcttttatctcttgctctcatcaaaatcccagcacctctcctctcgctgctaataaacttcaactcccatttctgcggcggcggctgcagcccagtggttcttgttttttctttatttattgtgatctgtaaaaaaaaatctaaagagtacaagaacaagggttaaaatcccagggtagacaagttgtagacatcacggctatacacggagtaactcggtccctaattcatgtccgggctgttcgacgtaagggctgtgtaatagagagcagcaacccttcgccgcacggtggacgtggtgctttaccctcggaaggcggtagcccttctgatctcctctttctagctggtggcggtttcttcttcttctgtttagtagtagtttagtgctattgttgttgttaattattattattattattattaaatctaaaataaaaggccagtctgcaaataagtaaaagtattagatctcgaataagcgaacaag

SEQ ID NO: 91 - DNA, 640 bp, Picea sitchensis
  source 1..640: mol_type = genomic DNA
cggtgaagaagtggtgagaagtgggtttgtggttaacgtttgttcagccgtcctctgatatctcttcagccttacacgtattttctggtataaacgtattgtcatctgaatctgaatttattagctgcgtctttatgaggttcgaaagcgggatctcctcagcttcagagtttactttctggacggaaagaaatctgtattcatagtttgtgaaggcagggaggggattcgatccggtgcagttgtggaggaaattcggagggtaacatcacggggactggtgtcgggagacgaaaataaggccattcaagaagggaaagaaaggtctggcaagttggggcaacacggctattaaacgtattttgcggttccagccccatgatcgcagaagaattctgcggagcaggaacgctggttgtgcactagagagtaccccgtcgccgcacggtgggagaggagccttgccctcggaaggcgggagcccctctgatctcctctttctcgcaggaggcggttgcgaccgagcccttttctgcctcgcccatccatttcttctgagcagaggagaaataatttgggagaggtagaggtggtttagcctaatttaggcgtcaatcaatcgctcatattaaccaccacc

SEQ ID NO: 92 - DNA, 253 bp, Physcomitrella patens
  source 1..253: mol_type = genomic DNA
cgcgtccatatgagcaaagacttctaccgtgcaggaatccaactgtcccgatctctctcttcctctctttccttgcacggtggtcggggggccgcccccagtgaaggcggtcgtccctctgacctatctgcacttgctgggggaggttttctctcaaactttcaccacggtgctgatccttaggggggggtgctagggagaattttctgctcaggtaacaggaagtgtctaggagactattcaacctgcaaaa

SEQ ID NO: 93 - DNA, 477 bp, Actinidia arguta
  source 1..477: mol_type = genomic DNA
gcaaaatcaacccttcatcttccacatcggcctcccaaacccacctcttctccacaatccaggcacaccttgagcggctggcgttgagggaatagagagagagagagagagagattttctgcttcgatcgggggtaaaacccggtgtttgacaagttgtagacatcacggctatactcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggtggcggtcacttcgctttctccgtctactagcttattaggtttattcttacttagtgagtaatttgtcctattatagttcgtaagttcgtcgaagatctgttgcttgattcgtctttcgttgctcgagtcttggtgtttttgcgttttctgagttcgag

SEQ ID NO: 94 - DNA, 655 bp, Actinidia eriantha; identical to SEQ ID NO: 81
  misc_feature 219: note = n is a, c, g, or t
  misc_feature 356: note = n is a, c, g, or t
  misc_feature 364: note = n is a, c, g, or t
  source 1..655: mol_type = genomic DNA

SEQ ID NO: 95 - DNA, 473 bp, Actinidia chinensis
  source 1..473: mol_type = genomic DNA
cacaaaatcaatccttcatcttccacatcggcctccaaaacccacctcttctccacaatccagacacaccttgagctgctggcgttgagcgaatagatagagagagagattttctgcttcgatcgggggtaaaacccggtgtttgacaagttgtagacatcacggctatactcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctactagcttattaggtttattcttacttagtgagtaattcgtcctattatagttcgtaagttcatcaaagatctgttacttgattcgtctttcgttgctcgagtcttggtgttttttgcgttttctgagttcgag

SEQ ID NO: 96 - DNA, 444 bp, Fragaria vulgaris
  source 1..444: mol_type = genomic DNA
aatacaccaccacataaccaaaacccactgccccattttctcgattctcattcgcatctgactaggaaggagagattttctgcgttgggtttgattccggtttggggtaaaacccgggtcgacgagttgtagacatcacggctatacacagagtttcacggccactcattcatgtccgccggactgtccgacgtaagggttgtgtaattgagagcaacccttcgccgcacggcgggcgtggtgctttgccgtccgaaggaggaagtccttccgacctccttttcctcgccggcggtggtgtccacgtttccgacttgcgtttctttttctagctttttgtagattcgggtttagctcaaagattattgtttcgcaagtagatcgtgtttgctgtcgttgctgtgctttgatttttcttttgagaagaacaagaacataacaaaa

SEQ ID NO: 97 - DNA, 600 bp, Solanum tuberosum
  source 1..600: mol_type = genomic DNA
ccatttgtatcggttcacacacagtaagcaagcgccattatccatctctacccccattggcaaacccacagccacaattttcctatcctccattatcccttctccaatttattatataaatacccacatctccctgctctttctcccttatcatcaacaacaacaaccaaatttcttctttttttcttccacagtagcaatctatcaacaaaacagagaccccattacaagaatcttggaattttagttttgggtttaagaggaaggggttattgtatttgcagttttgagggtaaagcccagtttaacaaggtgtagacatcacggctatacacaaagtaaaccgccgaccacttttacatgttccagcagtacgtcgtaagggttgtgtaacagctactaaccctgcgccgcacggtggacgtggcgctttgccctctgaaggtggcagtccttccgacctcctcttccttgccggcggcggttctttcctctccttctcctactagatatagttatacttaccgtagatctctagcttattacgtacagttctatctagtattctcttgattattcgaagaaaaacacaaaaagatg

SEQ ID NO: 98 - DNA, 499 bp, Chlamydomonas reinhardtii
  source 1..499: mol_type = genomic DNA
cagcttccaaactttgtctgtattatctgccacgcttgctcgagctgcattacctgagagtgcgacaaagcgacctcgcgtaaattgaatggcttgtgcggctactttctcccaacttcttcatgcatttgcgtgagccggtaaaaacggcgttctctgaggcagcccgcgttcagtcggctgcgtcgcagcctgcgaccgccaatcggtgctccggtggccgtggtgcagccccctcgtgcggtggcaagcccaaggacgctctgagcaaggcgggcggtggccagtaaccttcctcgcgcaacacaccgcgcgagcgcctgcggctgttggacagccagcagcgtgtgtcgaccccgcgccaggacaccggcagcgacgtcgacggctagtatcatctagcctttagcaactctagcctagaaacttagtattcgctcacgaaacttttaggagcttttcgtcgatcaacatcgcctcgctcgtcgccgcggacaca

SEQ ID NO: 99 - DNA, 480 bp, Zea mays
  source 1..480: mol_type = genomic DNA
aaaccaaacccagctcaggcacaaccccaacgcaccgccgccgctcgcctggcctcacctcgccggagaagagttataagacaggagaaccccgtccccggggcgcaggcctcggcagcttccgtgaccaccgcccaatcctgccacagctgccctccctcctcccctctgggcgtggccgagttgtaggcatcacggctagcgttgcagccccccggcgcggccccgcagctgcgcaggtggagcccacgggcaccatcgcctccgctgtcgcgtccagccccgctccgcacggcggccgcggggcgctgccctcggccggaggaagcccgtccgatctcctcttcctcgccggcggsgktcgcctctgagccgttcctgtctctcaccttcttcctttcctagcaattagtcccttctgctaacccccccggaccccgacgagttctcaccccgagagtaactgaccgacctaccggc

SEQ ID NO: 100 - DNA, 337 bp, Selaginella moellendorffii
  source 1..337: mol_type = genomic DNA
gcttttgattcaggtatggttgctccccaattcagcacatttcctttctttttccttgcagggtttattcttatgcactgactagcctggaagaaggctagtccgtgccaaaattttgacaaccacggcttcaattgagcttttgagtcgacaccccatcatttgttatggacacagtgtttccagttgtaagactctgctgagttccctctcttgccacggtggtcgaggggctagcccctcggaaggaggacacccttcagatcttacctttcttgctggtggtggccttcttctcggagcaccatagtgggggtttcaatcttattggctaaag

SEQ ID NO: 101 - DNA, 2130 bp, Actinidia eriantha
  misc_feature 556: note = n is a, c, g, or t
  misc_feature 1261: note = n is a, c, g, or t
  misc_feature 1750: note = n is a, c, g, or t
  misc_feature 1754..1755: note = n is a, c, g, or t
  source 1..2130: mol_type = genomic DNA
atcccaaaatatttgttcacttagaaaattaactgataaaataatgcaaactctcctttttgttctcctcttttgaattgacgtgacacatttatctttttaattttagataatttcgaattattgaaaaaaaattaaactgttttccaaataataattttttagaaataatgcaaataatagttttttaaactattttccaaatattttttttcaaaaataatgcattattaagaataatattaaaaaatatcttcaaatattaaaaaatatatttttgataaaatttaataatatataaaaataaatgaatgtttttgtttgcataaacaatttctaataaaatattttgcgaaaatgtttttgtttgcataaacaatttctaataaaatattttgcgaaaatgtttttgttcgcataaacaatttctgataaaattttttgcaaaactaaacctaacacaaatgggtagcatttttgcttctttaaaatcttggattccctaaattagacaaataaattgggacggatcaacatttattttcttcttaattwntttctctaacactacaacaaaataattaaatagataagaaaagagaaaaaggaacttgagaacccacccaacttttaaacattgcagttgggtccttccgtacgttgcagtggtcctccacaacgtccacatgaaccacatgggcgtggttaatacaacgcaccccactctctctctctctctctctctctcgataattgtctccattcgcagtaaaattaccaaggccactcgtcccacagtgcacaccacggccgatccacagccacactcaccaatcacctctctctctctctctctctctagaatttatttgttgctcttggagcaacacgtcactttttgacacgtggtggtcggatccaatcatctcacgccatccaagcactcagtttcatgtgtttgccacgtcaccacaacaattccaccacaaacccaggtaaacacaagactaacagaacctcactccgttaatgccatcttcctgtcgctgactcgcatgaaataccaccacttttggaaaccaaacgccagaaaagattactctcaccaatattctctatgaacaaagaaattgggttattatttattatttacaagaaataaatggcaccaaccaaatttaaaaagacgtctctgcagcgattttcacctcattttattttttgagcttttaggtgtctcgtccgaaaccgacgccttctntattatgcaatttttcactcttctttgccttctcagtcccgaaatgactattttcaggcaacatcatagggtgattgggttgtttagctatgtaggtacgaaatctaaaaatttgaatttgtaaagtttatgaatatttcatcgcatcgagtactggcggaatgttcacggggttaacaggatttgaactccgttatttctttcttgagtaaacggacgtggctgaatacacggacaaccaattaaatggtgtatgatatttcgtgtggagcaccacgcgtagaaagtgaggtgttgcgtcaagagcatcaaataatttctcctctctctctctccctcttctctatctatatatcccccaatctggcctctcctcacctcacccccaaagtctacacagaatcaacccttcatctcccgcataggcctcccaaacccacctcttctccacaatccagacacacctcgagtggccggagtttagagagnaganngagagagagattttctgcttcgatcggggggtaaaacccggtgtttgacaagttgtagacatcacggctataatcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctactagcttattaggtttattcttacttagtgagtaattcgtcctattatagttcgtaagttcatcaaagatctgttacttgattcgtctttcgttgctcgagtcttggtgttttttgcgttttctgagttcgag

SEQ ID NO: 102 - DNA, 1959 bp, Arabidopsis thaliana
  source 1..1959: mol_type = genomic DNA
ttttgatacttgtcctctcgaaattgatgtttactgacgcacgattattgggaatttgggacttagtggtgtaccaatagcatctcagctttcatggttacaaagaggccagtgaaagaggcttggtcaaccgcaattatgatccgttacttgcactgactaccaccgatccaccgccgtaactctttaaatcttaatgcaatactaattcgaattacatgtcgaggtcttaaaacatgataaatggttgtttaaagtaaaaaaagaaaagaaacaataaataaaacaaacggtgatggaaggagggacgtacgatcgtagtcgtagagactgccaaataaataatggaccactattatgtggttcaagaactatatccttttcttagattccgaatcttaacttgtgtttcttgccttgtttggttttttttttgtttattttgtagttttgtctcataaaataacgaacaaaaagcaatggtctaaatatttctcaatatatgtatttaaaaattatatcatagtttcgcaaaaaaacgaccgaacgaacgatggtagtcatttgcccaaattgagcctcgtagtagttgctacgaccacacccccgtcccatgaataaacacgaccatgtaaatattatctataaagacaacataaatttacaaattaatgttgaacagaaaaagtcaaaaagaaaatttcgttaatataataataatattctatgataatataaaacgtggcataacacatgacttcacatgacatcataagaagacatatgccacatgaactcttcatcgcctccatccttttagtctcgtttacatgcagcaaactacgatctacgattatatacaatgaaattcaaattcataatcaattggcattaaacatacgtatatcataattcataaggttaactaggtttagcaaatgttattctctttggataaacgtttagcaattgtttatctcaaatttaattgaaaatacttgtaagacacagttacaattagttctcaatttccaatctacacaagatttaaatctttctttagttcattctgatctatttttctctcaaagaaaatgtagtgctccaacttttctcccaagactaataaaaattttcttaattgcatgcaaaaatattttttccaactttaatcttatcaaacttagatctcattttatcttccgaataagatttttgttttggtcctttgcctaatcaaactaactaattagattagaagaaaaatacaattagacaaaataatatcgatgaaaaaataaaatccacaagaaaggacctaagaaatttcacgtccgaatcacaaccacagaaaaaaagagagaaatattagtatataattaaaattttgtcgtctgtcttctctggttcactcatctcctatctatttaaagcccattcgatatcctaaaacactgtatcatcaaaaaacacctcaaagaattattcattcaggcatcttctcaaatttttgtttgtgaaaaaaacccacatcaaaagatctctcatttattcgtttcgtttctgctgttttgagtgtcgggttcgttttagctgtaatctttttttccggcgttcgatttgaaaaaatccggggaacaggtgatcggaatcacggctatacacgggatatcacggggtgttagctcacatgtccatattgtccgacagaagggttgtttaatcgaaactaatcctttgccgcacggaggacgtggagctctgccgtctgaaggcggcagcccttccgatctcctctttctcgccggtggcggttccagctttaacttcttttcctttaggttttaggagttagggtttgttagtgttttttccttcttctttttttggtgctcttgaatcgcttttttcttgggggaagttttttcttttgctcttcgaaatttgtcttttttgaga

SEQ ID NO: 103 - DNA, 1161 bp, Arabidopsis thaliana
  source 1..1161: mol_type = genomic DNA
acgcgtgatgaatcaatcattcattcacgccaacgtaaaaaaaaaaaaaaaaaaaaaatttaactaaaattatacggatgatttatttacatggacgctgattaaaaaaggtcggtccaattaattatttacttaataattaattaaagtgttgtgtgtatttttggattcccataatgttttgaggggtatataaaggagattattttttctattttcaatgtctacgaataaaatggaacatcctcgttccaatatatactgctgttttattactaaggtttataattggagtgtataaaaacactccaaaataatatcatgaataatgtttttatacgtataatacataaaataaaatctctttcctcatagctggtcctctgattactatcaaacatatgattcaattgcaaatttgcaattattaatgcaagaagaatgagtggaacattaaagttagatcaaaaactcttcattacaaacataaataaattcattttggtgctttctaaattaatttattgatcattttttggtatgtagctaggggttaactcatcaggaagaacattacctagatgatgtcattgtcaaagtcaataggttgacttatccgatggatactaatccaacgggtcaaaaagtgccaaatcgggtcaaaatcaaaaacctcaaagatgacccgtgaacacttgtttttatcttcctcacaaaatccacacgtccaaatcacaacctcaaaatctcacacatcaaacacgtgaatttgctctctttctggttcactcacctaccattactataagtctgaaagagtgattgaaacccacctcgaaaaatctatccttttttttgttttccttctccggcgaatccccggggagattggtaatcggtaatcacggctatttacgggataaagccacggcctttgagctttcatgtccaacgaaagggttgtttaatcataactaatcctttgcctcacggaggacgtggagctctgccgtctgaaggcggcagtccctccgatctcctcttcctcgccggaggcggttccacaaatagccactaaccctaaccctttttctaattaggtttttagttcttagagtcctgtattaatctgttatttcgagattataatatttgtgagca

SEQ ID NO: 104 - DNA, 2003 bp, Malus domestica
  source 1..2003: mol_type = genomic DNA
gacgagatcagcacgatgttggaggagttagaggcctgacagaaaaaaaaaayattaaaacaggcgtctaacgtcagatccacctcaggctctcgggccggcgagaatcgacgggcctggggctcggggttgctcaggctcccttgccagccaacgctggacttcctacctcgggcaaccaagctcggggttgttcaacatctttctctctctccctctttcacatattcccatcaattttttctctcttctttcacctcacccatccaaaactacaatattcggttttattttttttatggaaaataaatttaktaaagagaaaatcatctcgaacaacatgttccaactccmaattcaattaaagttcaaagcatctaactccgaagcawggaggcaataattctaaaagtggggattggacaccaacaccccaacgtaagcaagcttatcaaccacaaaattgattttattatgtttccagaagaagtcgattgcaacacttggaaaataaaccaacagctacggaaaatcaaaacaaacacaacagaatttctatataaaaatgcttgtacacattgccataccctaattttcaccatgttatgaaattttcaatctgaattttagcaattgtaaatttccgaaatcttgaacaattttcttcatttcaacaaaagagaaatgttgtaaacgggaattgacatatttttaatccgataaacyctttctctktctcaattaattcaagagaactttaacgaaaaactytcgttactgttcaytttaataaaaaatcacatttttaaactaaaaagtcaatcmtkgtactatwcactttaccctttattttgtccttatcattaaaacttttcragtttttttcattagtttcyttttaattcaaarcsgtttaagtattatatatttcaaccctcattttcttgcatataaagtcaagtcttttttccctcsatccgtacacaaagtggtccatatacggcaatcataagtaccagtgaacctcaatctctcacacattttttttctcaattattgtcccaaaaaaggacccggcttttcttcgttctcactttctatacactttcaactcttcttaatttatttatgcarttacgsttaagttatgttaatattttatattaatttttttttataaarataataaaacaaaaaayaataataatataaaatattaatrtaayttaaccgtaatygcayaaataaaaaaaataasaatgtattaaarrtgggaggacaaagaagtcaggtccctagaaaatacaccatcaaaccaaccaaggccacgaaaccccccaaaatacaaacacaaaccccacggcacatcctctaccacaaatccacttttcctccccatgcctataaatccaccccctcatctcctctccacacccccacaaccacaaaccccacctgctcccccaattccccagtacccatttgtgtttttcgattcagttcgaaaccaggcggtcttcacctttctggttgttttcctatctgggttttaaggaggaagaagaaaggaaggcgttttgatcattttcttttcgaatttcttttggggtaagacccaggttcgacgagttgtagacatcacggctatacccagagctcctcggccgctcgttcatgtccggggtgtcggacgaaagggwtgtgtaattgagagcaacccttcgccgcacggcgggcgtggtgctttgccttccgaaggcggtagcccgtctgacctcctcttcctcgccggtggcggctccccttcctctgtttttctcttctgcttttattagttttatttttatagagtttcttgcttagatttttagagattttttgttctataaagcgctcgagtagatcgtatttttgttttcgggggttttttttttttgtggtgtttgatttttactgagaaatcgagaaaaaaagrgagagagagagagagagagaaagaaggc

SEQ ID NO: 105 - DNA, 2000 bp, Malus domestica
  source 1..2000: mol_type = genomic DNA
tgcccttctcaataacaataacatagaatgatactttgcakaactcaaayagactagggctcatctggactrcttttaaaatgattaaaasygtttttkgtgaaaatattttaatatcaatcyttagtaaaaattcaagtggaycctcaaaaacayttggagtgcttcctataagaatcatattggtgcttcttccaacaaacaattgaagtgctttttgaactcaaaattcatttcaccaaaaacactttcaaatattactatgcttcctcttttgtagttaagttcaaatcatctatcgctatgtccaaaaacaaaacattaaaaaaatgtgagatcatattaaaaaaaagaaaattggaactttgtcttcttgaactaaaatatcatgagtactcttgaacttgtcaaatatagaacaatggccatctagataacttgttaggacttacataacttatttgctcctttaattctttattttttatgaaagttctaactgtrttatctaaagaggttattgctccacattacgacaagctcaaaaaattcatcaaaattaattaggtataaaatctcttattgttggttacatgttcaatttactaaacttcagcgttggataccaatatatatgtgtttaggtacaagctttagaaacaaggataatgcagccatstagtgaaactggcatgaagaccccatgtttttatggtcaccgcccacacccatgtgagcaagacatggacaatggataaggtgattgtccttggatattmtggggcatgtcccttaattttaaggacgacgactgctactagtctactaccaccactttttgctttgcatagtactttgggccctgtgcgtccttgaatgtyctggtgcatttactataactttaaggacgacgacttagactactatttatttattttaattaactgttattaagagaaaaagatgggagttcgaactattacaaaaaataaaagagmgggtttagaattcataatgcatagataaaaatttaacattctattcattaagatattgaactacatgcactactaatacttctttagttgggragttgcaggcaaaacaactcgtgcatgtgaacacattgtgatttggttttctctcgagtacaacacactatcattttggttttctctagagttttagcgagttgtcatgaaattagtttttatttttatttgttattcaacgacatrgttacaccaagtgtagatgcataatttgggaaaaccayataataagctaactataacaatttaatatcaaatgcrtcatctatgaagttgagccttgagctcctacttatgtacaaatgaaaatgaataccgtartactaaatgactaacttagacatcaagaaattgataaaataaaawttaaaaaaaaaaactacaagcatgatgatgaggaatgaggatcctatctgaatcctctttatgaggactctaaagattctccaatcacgtccgtttattatagatcgtacgacttgtttttgttagatgttatttatattcaatttaaaaaaaattacaataatatataatcacatgatatacaataaaatagatataattaaaagattcctataattaaagaatcctcacaaagagatttccgaacgctctttcgaccatgagggtagttaggctcctaaatacttgaaaggtacagtacatcatcatggtaggtttgaagttataagtccacctcatccaaaacacagcatatagaatgattccctcatggatcccgaatcctaaatttttatttcaattaaaatttgtgtgcttgctttgtggaatgcggaccgtcttcttgtaaatttcgtaggaagactcggctccaattttttawgattaaaatccgctgccatggtcaattcactgtcgtctaattatcaacaggcagccttccaacccacgtgtttgtttgcacgcattagaatattcactgtc

SEQ ID NO: 106 - DNA, 2018 bp, Malus domestica
  source 1..2018: mol_type = genomic DNA
ccgaggacctcagaagcgagggggcaatgtttgggcccaaaataacagtttgggccgagtgtagggtcattctcggcccggaaggccttacgacaagaatactatggatcgttcagtacgtgggcatccaaacctaggtcagctaggtcataacgcaagaggtcgaatcctagtgcaataaggagtctcggcaggacccggaagtgaatccggctcagttagggactaggttcacagtcctagtgaaggtaggactggtcgagttgatgctgatcaggggaagaaaacctagtccgagtaggtttttaactcgaccttggggggagccattgctataaatagaaaaggttgcacatccttcaatgacccctgcaaatcaatacaaaattgccctgcgcaaattctcacaacttgtgatttttctttttccttttttcgctgacacatcttccgttggcatcaacagcactgtggaagcaaccggtgatatcttaagtcggcatagatagctctgtcaccgtagagttagtcggtctcgcagtatcttccgttggcatcaacagcactgcggcgagaayggttgattacctatccaagtctcggtcragaagggtttctaaatccttattggtcgaggtcatctcatcagccttctcggcgragtgaggtgttacagttattacattcggcacattgaaagccgaatttgatattgaacttcktaagaatagtaaccttgtcttcaggttcgagagcccaagaggycgagacgtgttcctttctcggccgcaatcgcaagacgcagaagtcagtagcgcgacccaacgcaacatcaayaaatttactcmtcggccgagctcggcckacgagttggcacgccccgcattcaccgaaggacgtagttagctcattaattactcggcctgcgcgccacgtaggctttgtagtttctagggtcaacaaggtgaaattcttgaatttatgaaagacaaacaattacaaaaatgtttgctaatgatgttttcattaattaagaatgaaaattggaaatccgaagatgatcacataccaatgggctttgtgatgtcatttatgttttagaccgatgcccctctcgaatgaggatcctctctggatcctctctgtgagaatcatctccggatcctctttttaagaatctcgagaattctcaaatcgtgttcgtttatcgtacatcatgcgatcaagtttcttcagttattgttcatgtttaattttaaataaaaatatttaaaatgatttttgatcgaacaatgtacgataaaagaacacgatttaagaattttcagaattatagtaaagaatatccgaagaggatcctcattcgccccctctatacccaaaaaaccaataaatagatccgaccgaatatccaaaatccaaaaacagttgcgtaatatctttacacatgaacggttgggatcggtcaaaggacggaaaaccagaacacacgtgtccgtctccgttcgtcccagtagcagaccgtagaccggaactctgtatcgcgaaatcaaaagacgtcacgatatagaagaagaagagaaagagaaacggagatagatatttgatacagattttccgatcgcacaatcatcgacattctcgaccaatttgaagactcggaatcgctggtccggcgagggctggcttcgtagatgcaatcacggctctaatcaaagcaatcactagtattaagatttcgagaggcagcagagaatgcatgagagccttccctaattccctgcttgcatgcccttcgtttcacggcggtcgcggtgccctaccctccgccggtgggcatccatccgatctcacctacctggccggcggcggttgcttctgaaaacgacgaccgcctgtgtttccccttctgggttttgagtgtacttcgttttttattaagcagtaatcaatggttacggttaagcagcttcaagatggtaactttacgtcgcaattccctccattttctctgcaag

SEQ ID NO: 107 - DNA, 2000 bp, Actinidia chinensis
  source 1..2000: mol_type = genomic DNA
ttggtgtataaagatgagtaaatagggatcccggcaaatggtctccacaaaagtgtagtgtcttaggggcactatagagattccaaatctccctagatattttgttccacagtatatgatccacaatgagccatgtattttatataattttatttatttttttataattactcataggttatgtttgaatgctagtaatcacaaggagaatagcaatatgatttcaaatctttggaattttaccatttggataaagtaggaataagaatataatatttattgatattttaaattagattcataaagtaacgaggaatctgattctgcaaagatcctaattaatttcaaatttctccgtcatttttttataaatttgataacaaatataaatccttttttaaaatattatttcgtgattaagattctgaacaaaggcaagtacttgaataagattaactctttcattaaattattaaaaattatattattaataacgaaaacgatcatccaaacacagcataggggaatggatatattccgtggtatcaatgaaatttgcaaatgtgcgtgagaacccaactttattcatggtgaaaataagcgaataggaagggcaaatttcgccaatggtctctgtagtttaacaaaagtttcactttggttcttataattttaatataaacaattaaaattttgaacttttaattttatttttatatagttgttctatctataatcgttaagaatttgaatggaaagtgagaacatagagctcacgtaggccctttttagaggataattttactactatttttgcttataatttggttaaaattttactttagtccttataaataatcacgagttttcacatgctttttctccatttatgttattaatttttattgacagagggaagctacattgaaatccaattgaaactttagagatataactgtccaaattgaaaataaaggaactaaagtgaacttttaatcaaattacaaagacaagatgaaattttcccgaataacaacttagcacattggcatattcgtcgctctcttgtgtatagacaacactgccctcaactattcactgttactacaaaattgccacacattcaagctaaagcatcaacccactatgatactatgatgctgcaacacagccttgaatattaggtatttattgtagttgagtttatatttaaattgaatttttattaaaaattaaatatttaaaaatagaagttaaaaaatattaaaataaacttttttaaaatttaactttttaattttttagaagttatttatagtccataagtagaaaaactataataaacacagactttaaggcaaaataaatcaaaagtttctactaataagtaacaacaaaacatgggtgtttgatagagtgaacttacactataattgccaatttaaaaataaaatttaaaaaattttaaaataaagattttaagaatcagctatttttcaactttcgtagtttgttttggcccataagtagaagtagacgcaaaaactgtgctaaacactttggcgcaacttttaagacaaaaatggctataagttgtatgccaaacatgcaatattcgctctaaaattgccaagtatgtcaccattggtcattccgattaaacccaggggtacttttgtctttaaatttctataatttatgtactttttcattttttttttgctttttgttataattataaattaatgatgtccactctgttcttccatgtaccacctgaaagatcaagaagcaagatatatatattgttattctaccattctctctctctctctctctctctcatcagacaattctctttcaaaaaacaaaggaatggccaaaaacggtacggccccattaatctctcttctgtgtgtgtgtgtgtgtgtgtgtgtgtgtaaatgtgtatattgtgtacaatgtatttgttatctgtgtttcgatggatttgttgtgtg

SEQ ID NO: 108 - protein, 28 aa, synthetic construct
  REGION 1..28: note = Construct
NPSPHGGRGALPSEGGSPSDLLFLAGGG

SEQ ID NO: 109 - protein, 20 aa, synthetic construct
  REGION 1..20: note = Primer
TATCCGCTGGAAGATGGAAC

SEQ ID NO: 110 - DNA, 20 bp, synthetic construct
  misc_feature 1..20: note = Primer
  source 1..20: mol_type = other DNA
tccacctcgatatgtgcatc

SEQ ID NO: 111 - DNA, 246 bp, Cucumis sativus
  source 1..246: mol_type = genomic DNA
gggtaaaacccgggttcgtcaggctgtagacatcacggctatacacgtagtttcccggtcgttctttcatgtcagggctgtacgacggaagggttgtataactccgacaaacccttcgccgcacggcggacgtggtgctttgccttccgaaggtggtagtccttctgatcttctttttctcgccggcggtggtttctcttgcttcttctcttcttcgtattagctttccgtttgtgttttagctct

SEQ ID NO: 112 - DNA, 254 bp, Actinidia eriantha
  source 1..254: mol_type = genomic DNA
gggtaaaacccggtgtttgacaagttgtagacatcacggctataatcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctactagcttattaggtttattcttacttagtgagtaattcgt

SEQ ID NO: 113 - DNA, 253 bp, Actinidia arguta
  source 1..253: mol_type = genomic DNA
ggtaaaacccggtgtttgacaagttgtagacatcacggctatactcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggtggcggtcacttcgctttctccgtctactagcttattaggtttattcttacttagtgagtaatttgt

SEQ ID NO: 114 - DNA, 255 bp, Actinidia eriantha
  misc_feature 49: note = n is a, c, g, or t
  misc_feature 57: note = n is a, c, g, or t
  source 1..255: mol_type = genomic DNA
ggtaaaacccggtgtttgacaagttgtagacatcacggctatactcggnagtttctncggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctactagcttattaggtttattcttacttagtgagtaattcgt

SEQ ID NO: 115 - DNA, 253 bp, Actinidia chinensis
  source 1..253: mol_type = genomic DNA
ggtaaaacccggtgtttgacaagttgtagacatcacggctatactcggagtttctcggccgctcatacatgtccggtctgtacgacgcaagggttgtgtagtcgagagcaacccttcgccgcacggcggacgtggcgccttgccgtccgaaggcggtagcccctccgacctcctcttcctcgccggcggcggtcacttcgctttctccgtctactagcttattaggtttattcttacttagtgagtaattcgt

SEQ ID NO: 116 - DNA, 254 bp, Malus domestica
  source 1..254: mol_type = genomic DNA
ggtaagacccaggttcgacgagttgtagacatcacggctatacccagagctcctcggccgctcgttcatgtccggggtgtcggacgaaagggttgtgtaattgagagcaacccttcgccgcacggcgggcgtggtgctttgccttccgaaggcggtagcccgtctgacctcctcttcctcgccggtggcggctccccttcctctgtttttctcttctgcttttattagttttatttttatagagtttcttgctt

SEQ ID NO: 117 - DNA, 247 bp, Fragaria vulgaris
  source 1..247: mol_type = genomic DNA
ggtaaaacccgggtcgacgagttgtagacatcacggctatacacagagtttcacggccactcattcatgtccgccggactgtccgacgtaagggttgtgtaattgagagcaacccttcgccgcacggcgggcgtggtgctttgccgtccgaaggaggaagtccttccgacctccttttcctcgccggcggtggtgtccacgtttccgacttgcgtttctttttctagctttttgtagattcgggttt

SEQ ID NO: 118 - DNA, 256 bp, Arabidopsis thaliana
  source 1..256: mol_type = genomic DNA
tgaaaaaatccggggaacaggtgatcggaatcacggctatacacgggatatcacggggtgttagctcacatgtccatattgtccgacagaagggttgtttaatcgaaactaatcctttgccgcacggaggacgtggagctctgccgtctgaaggcggcagcccttccgatctcctctttctcgccggtggcggttccagctttaacttcttttcctttaggttttaggagttagggtttgttagtgttttttcctt

SEQ ID NO: 119 - DNA, 245 bp, Arabidopsis thaliana
  source 1..245: mol_type = genomic DNA
aatccccggggagattggtaatcggtaatcacggctatttacgggataaagccacggcctttgagctttcatgtccaacgaaagggttgtttaatcataactaatcctttgcctcacggaggacgtggagctctgccgtctgaaggcggcagtccctccgatctcctcttcctcgccggaggcggttccacaaatagccactaaccctaaccctttttctaattaggtttttagttcttagagtc

SEQ ID NO: 120 - DNA, 256 bp, Populus trichocarpa
  source 1..256: mol_type = genomic DNA
ggttaaaatcccagggtagacaagttgtagacatcacggctatacacggagtaactcggtccctaattcatgtccgggctgttcgacgtaagggctgtgtaatagagagcagcaacccttcgccgcacggtggacgtggtgctttaccctcggaaggcggtagcccttctgatctcctctttctagctggtggcggtttcttcttcttctgtttagtagtagtttagtgctattgttgttgttaattattattatt

SEQ ID NO: 121 - DNA, 252 bp, Glycine max
  source 1..252: mol_type = genomic DNA
ggttgaatccctgggttgaagcgttgcagacatcacggctattctaagagtgtctcgctctctcattcatgtcccaactgtacgacggaggacaggttgcgtaactgccaccaacccttcgccgcacggtgggcgtggtgctttgccttctgaaggtggtagcccttcagacttgctcttcttagctggtggtggttctgctgtcttctagcttcttcttaactcttttttctttttactacttttaagcta

SEQ ID NO: 122 - DNA, 253 bp, Medicago truncatula
  source 1..253: mol_type = genomic DNA
ggttgattccctgtattgacacgttgtagacatcacggcttttcttagagtttatcggtcactttcacatgcccgaactgtacgacgtaagggttgttgcttaactccgactaacccttcgccgcacggtgggcgtggtgctctgccttctgaaggtggtagcccctccgatcttctcttcctcgccggtggtggtgtctttgctccttcttccttctaatttcttgtttttagtttaactttctttagattt

SEQ ID NO: 123 - DNA, 252 bp, Solanum lycopersicum
  source 1..252: mol_type = genomic DNA
gggtaaagcccagtttaacaagttgtagacatcacggctatacacaaagtaaaccgccgaccacttttacatgttccagcagtacgtcgtaagggttgtgtaacagctactaaccctgcgccgcacggtggacgtggcgctttgccttctgaaggtggtagtccttccgacctcctcttccttgccggcggcggttctttcctctccttctcctactagatatagttatacttactatagatctctagctta

SEQ ID NO: 124 - DNA, 252 bp, Solanum tuberosum
  source 1..252: mol_type = genomic DNA
gggtaaagcccagtttaacaaggtgtagacatcacggctatacacaaagtaaaccgccgaccacttttacatgttccagcagtacgtcgtaagggttgtgtaacagctactaaccctgcgccgcacggtggacgtggcgctttgccctctgaaggtggcagtccttccgacctcctcttccttgccggcggcggttctttcctctccttctcctactagatatagttatacttaccgtagatctctagctta

SEQ ID NO: 125 - DNA, 258 bp, Vitis vinifera
  source 1..258: mol_type = genomic DNA
gggtaaaacccttgttcgacgagttgtagacatcacggctatacaaagaattccgccgcctctcatacatgtccgggcggtacgacgcaagggytgtgtaattgagagcaacccttcgccgcacggcgggcgtggcgctttgccttcagaaggcggtagtccctctgatctgctcttcctcgccggcggtggttccaacgctttcctctgctagtttaggcttatattctgcataatatagctactgtctttaggatt

SEQ ID NO: 126 - DNA, 682 bp, Solanum lycopersicum
  source 1..682: mol_type = genomic DNA
atttgttcggtatactgtaaccccctgtttgcgattggccttgtagccccgttttacatcttccagagactccatttgtatcggttcacatacagtagcaaagcgccattatcttactctaccccattggcaaacccacagccacaattttccaatcctccattatcccttctacaattttctatataaatacccacatctctctgctctactcccttattatcaacaacaaccaccaaatttcttcttttttttcttcgatagtagcaatctatcaacaaaaacagagaccccatcacaagaatcttggaattttagtgttgggtttaagaggaaaaggggttattgtattttgcagttttgagggtaaagcccagtttaacaagttgtagacatcacggctatacacaaagtaaaccgccgaccacttttacatgttccagcagtacgtcgtaagggttgtgtaacagctactaaccctgcgccgcacggtggacgtggcgctttgccttctgaaggtggtagtccttccgacctcctcttccttgccggcggcggttctttcctctccttctcctactagatatagttatacttactatagatctctagcttattacgtacagttgtatctagtattctattgattattcgaagaaaacacacaaaaagaagtaaagcc

SEQ ID NO: 127 - DNA, 472 bp, Solanum tuberosum
  source 1..472: mol_type = genomic DNA
taagggggtgcttatataaagttggggagtctaccaatgagacgaactcattgaccaaatacgtctgcaggagaaagaccaccggagcaccaaacgccacccaacaaccacccattaaattcttccagaaaaaaacatcttcctcaaaattatcgatgaaggatcgttccttagtagttgttcgttgatcctacaaattcaatcacggctcttcttggatctttcgtttgtattctcacaattcatcatcaccgcaaagtgttgacccttaatccaactcttctggtggacgataagcaccggaccccttcccctcacggaggtaggggtgcctcacccgctgaaggcggttgcccctccgatctcctcttcctcgccggcggcggtccaattcttcctttctctttctccttctcctaatttttcgtgtaagaattgtatttttgattatccatccaagaacaggaccgcc

SEQ ID NO: 128 - DNA, 607 bp, Malus domestica
  source 1..607: mol_type = genomic DNA
ccacggtacaccctcagccacgaacaccccttcttctccccacacctataaatccaccccctcatctcctccccacacccccactcacttcagttcgaaacaggcgatcctcgcctttctgggttgtttcctattttatctgagggagaagaaaggaaggtgtttgatcaattttttggtatatttttaggggtaagacccaggttcgacgagttgtagacatcacggctatacacggagctcctcggccgctcattcatgtccgggctgtccgacgaaagggttgtgtaattgagagcaacccttcgccgcacggcgggcgtggcgctttgccttccgaaggcggtagcccctccgacctgctcttcctcgctggtggcggttctgcatcctctgtttttctcttctgcttatattagcttttttagactttcttggttagattcttaggagattttagagattttttttcttctataaagcgcacgagtagatcgtattgttgttttcggggggttttgggtttggtggtgtttgattttactgagaattaagaaaaaataaaaggaaaaaaaagagagagagaaagaaggggagggagcatgcc

SEQ ID NO: 129 - DNA, 183 bp, Solanum lycopersicum; identical to SEQ ID NO: 44
  source 1..183: mol_type = genomic DNA

SEQ ID NO: 130 - DNA, 213 bp, Solanum tuberosum
  source 1..213: mol_type = genomic DNA
acggctcttcttggatctttcgtttgtattctcacaattcatcatcaccgcaaagtgttgacccttaatccaactcttctggtggacgataagcaccggaccccttcccctcacggaggtaggggtgcctcacccgctgaaggcggttgcccctccgatctcctcttcctcgccggcggcggtccaattcttcctttctctttctccttctcc

SEQ ID NO: 131 - DNA, 192 bp, Malus domestica
  source 1..192: mol_type = genomic DNA
acggctatacacggagctcctcggccgctcattcatgtccgggctgtccgacgaaagggttgtgtaattgagagcaacccttcgccgcacggcgggcgtggcgctttgccttccgaaggcggtagcccctccgacctgctcttcctcgctggtggcggttctgcatcctctgtttttctcttctgcttatat

SEQ ID NO: 132 - protein, 61 aa, Solanum lycopersicum; identical to SEQ ID NO: 4

SEQ ID NO: 133 - protein, 71 aa, Solanum tuberosum
TALLGSFVCILTIHHHRKVLTLNPTLLVDDKHRTPSPHGGRGASPAEGGCPSDLLFLAGGGPILPFSFSFS

SEQ ID NO: 134 - protein, 64 aa, Malus domestica
TAIHGAPRPLIHVRAVRRKGCVIESNPSPHGGRGALPSEGGSPSDLLFLAGGGSASSVFLFCLY

SEQ ID NO: 135 - protein, 28 aa, Solanum lycopersicum
NPAPHGGRGALPSEGGSPSDLLFLAGGG

SEQ ID NO: 136 - protein, 28 aa, Solanum tuberosum
TPSPHGGRGASPAEGGCPSDLLFLAGGG

SEQ ID NO: 137 - protein, 28 aa, Malus domestica
NPSPHGGRGALPSEGGSPSDLLFLAGGG

SEQ ID NO: 138 - DNA, 84 bp, Solanum lycopersicum
  source 1..84: mol_type = genomic DNA
aaccctgcgccgcacggtggacgtggcgctttgccttctgaaggtggtagtccttccgacctcctcttccttgccggcggcggt

SEQ ID NO: 139 - DNA, 84 bp, Solanum tuberosum
  source 1..84: mol_type = genomic DNA
accccttcccctcacggaggtaggggtgcctcacccgctgaaggcggttgcccctccgatctcctcttcctcgccggcggcggt

SEQ ID NO: 140 - DNA, 84 bp, Malus domestica
  source 1..84: mol_type = genomic DNA
aacccttcgccgcacggcgggcgtggcgctttgccttccgaaggcggtagcccctccgacctgctcttcctcgctggtggcggt

SEQ ID NO: 141 - DNA, 276 bp, synthetic construct
  misc_feature 1..276: note = Consensus Sequence
  misc_feature 3: note = n is a, c, g, or t
  misc_feature 14: note = n is a, c, g, or t
  misc_feature 32: note = n is a, c, g, or t
  misc_feature 51: note = n is a, c, g, or t
  misc_feature 53: note = n is a, c, g, or t
  misc_feature 60: note = n is a, c, g, or t
  misc_feature 65..67: note = n is a, c, g, or t
  misc_feature 69: note = n is a, c, g, or t
  misc_feature 85: note = n is a, c, g, or t
  misc_feature 87..89: note = n is a, c, g, or t
  misc_feature 99: note = n is a, c, g, or t
  misc_feature 104..106: note = n is a, c, g, or t
  misc_feature 117: note = n is a, c, g, or t
  misc_feature 125..127: note = n is a, c, g, or t
  misc_feature 153: note = n is a, c, g, or t
  misc_feature 162: note = n is a, c, g, or t
  misc_feature 165: note = n is a, c, g, or t
  misc_feature 180: note = n is a, c, g, or t
  misc_feature 211..215: note = n is a, c, g, or t
  misc_feature 217: note = n is a, c, g, or t
  misc_feature 230..231: note = n is a, c, g, or t
  misc_feature 233..234: note = n is a, c, g, or t
  misc_feature 240..241: note = n is a, c, g, or t
  misc_feature 244..246: note = n is a, c, g, or t
  misc_feature 252: note = n is a, c, g, or t
  misc_feature 255..256: note = n is a, c, g, or t
  misc_feature 260: note = n is a, c, g, or t
  misc_feature 263: note = n is a, c, g, or t
  misc_feature 266..267: note = n is a, c, g, or t
  misc_feature 272..274: note = n is a, c, g, or t
  source 1..276: mol_type = other DNA
ggntaaaacccggngtttgacaagttgtagancatcacggctatacacggnangtttctncggcnnncnctcatacatgtccggncnnntgtacgacgnaaggnnngttgtgtaatngagagcannnacccttcgccgcacggcggacgtggngctttgccntcngaaggcggtagcccntccgacctcctcttcctcgccggcggcggtnnnnncnctttcttcttctnntnncttctnnttnnnttattnttnntttngtnganntttannntt

SEQ ID NO: 142 - protein, 58 aa, synthetic construct
  REGION 1..58: note = Consensus Sequence
TAINNVNRNLIHVRNAVRRNNNKGCVIESNNPSPHGGRGALPSEGGSPSDLLFLAGGG

SEQ ID NO: 143 - DNA, 12 bp, synthetic construct
  misc_feature 1..12: note = Construct
  source 1..12: mol_type = other DNA
gatctatagatc

SEQ ID NO: 144 - DNA, 2191 bp, Arabidopsis thaliana
  source 1..2191: mol_type = genomic DNA
ctggttcactcatctcctatctatttaaagcccattcgatatcctaaaacactgtatcatcaaaaaacacctcaaagaattattcattcaggcatcttctcaaatttttgtttgtgaaaaaaacccacatcaaaagatctctcatttattcgtttcgtttctgctgttttgagtgtcgggttcgttttagctgtaatctttttttccggcgttcgatttgaaaaaatccggggaacaggtgatcggaatcacggctatacacgggatatcacggggtgttagctcacatgtccatattgtccgacagaagggttgtttaatcgaaactaatcctttgccgcacggaggacgtggagctctgccgtctgaaggcggcagcccttccgatctcctctttctcgccggtggcggttccagctttaacttcttttcctttaggttttaggagttagggtttgttagtgttttttccttcttctttttttggtgctcttgaatcgcttttttcttgggggaagttttttcttttgctcttcgaaatttgtcttttttgagaatgttgaaaatcaaaagagttccgaccgttgtttcgaactaccagaaggacgatggagcggaggatcccgtcggctgtggacggaattgcctcggcgcttgttgccttaacggggctaggcttccattgtatgcatgtaagaatctggtaaaatccggagagaagcttgtaatcagtcatgaggctatagagcctcctgtagcttttctcgagtcccttgttctcggagagtgggaggataggttccaaagaggactttttcgctatgatgtcactgcctgcgaaaccaaagttatcccggggaagtatggtttcgttgctcagcttaacgagggtcgtcacttgaagaagaggccaactgagttccgtgtagataaggtgttgcagtcttttgatggcagcaaattcaacttcactaaagttggccaagaagagttgctcttccagtttgaagctggtgaagatgcccaagttcagttcttcccttgcatgcctattgaccctgagaattctcccagtgttgttgccatcaatgttagtccgatagagtatggccatgtgctgctgattcctcgtgttcttgactgcttgcctcaaaggatcgatcacaaaagccttttgcttgcagttcacatggctgctgaggctgctaatccatacttcagactcggttacaacagcttgggtgcttttgccactatcaatcatctccactttcaggcttattacttggccatgcctttcccactggagaaagctcctaccaagaagataactaccactgttagtggtgtcaaaatctcagagcttctaagttaccctgtgagaagtcttctctttgaaggtggaagctctatgcaagaactatctgatactgtttcagactgctgtgtttgccttcaaaacaacaacattcctttcaacattctcatctctgattgtggaaggcagatcttcttaatgccacagtgttacgcagagaaacaggctctaggtgaagtgagcccggaggtattggaaacacaagtgaacccagccgtgtgggagataagtggtcacatggtactgaagaggaaagaggattacgaaggtgcttcagaggataacgcgtggaggctccttgcggaagcttctctgtcggaggaaaggtttaaggaggttactgctctcgcctttgaagccataggttgtagtaaccaagaggaggatcttgaaggaaccatagttcatcagcaaaactctagtggcaatgttaaccagaaaagcaacagaacccatggaggtccgatcacaaatgggacggccgccgagtgccttgtccttcagtgaacaatatggtgacttggtggtttgtatgtataattaaaagcctaaataagcaaactctctttgtagttgcatttgaagcttcttggtttatgtatgatggttgtgggcattttgtgcctagacttctggttctttgttttttgttatgagttggtgtttatgaattatatatgttcttcactaatatgattattatttgtatagatttgattattcaaaggatatgtcttaagaaggggttcaggttcagccaactaattacttgtaaccaagtctttctaccatcaacaaccatgttaagattca

SEQ ID NO: 145 - DNA, 2459 bp, Arabidopsis thaliana
  source 1..2459: mol_type = genomic DNA
cgggtcgggtcggttcgtttcgggtcggttcgacccgtttttgctcccgccggtcgtccgcgagtcaatctctctttctatctataaaagcgagccaaccaaccttctcctccacccagccaaccaaacatccatcgtcgcccgcatcgcctgcaaaaaaaatctctccttttacctccgccatcgcccgcttcaaatcctaggaaaatccgaacccaagcttctcgattgttcggtcggtcggtcggtcgaaggaaatcgcggcgagtgttgtgtttgtcggggggtaaaacccggggcagacgagttgaggacatcacggctatacacggcgtagcccggccactcatccatgcccgctccgttcgacgccgcaagggttgcctgatcgccgcctcctgcgtcggcaacccttcgccgcacggcggacgaggcgccctcccctccgaaggcggcagcccctccgacctcctcttcctcgccggcggcggtccctcctccctcctctactgctagattattctttcctataggagtcttcgttttcgtccctagctacgacgtattccgccgcgtgtaacgagcaagtcgctgttcttttttttttttagatctcgttttttgtgtttttttcctcgtacttaaaaaaccggggagggaagatgatgctcaagattaagagggttccgaccgtcgtctcgaattaccagaaggaggaggttgaaggcgggcgcgtcgggggttgtggccgcaattgtctcaacaagtgctgcgttcaaggggcgaaattccctttatacgctttcaagaaggagaaggaagtcccaggcgacagggctttgcttgggcatgtggagagggagcctcccgtcgcttttctcgactctcttctgcttggagagtgggaggatcggatgcagaggggtctgtttcgctatgatgtcactgcctgtgaaactaaggtgattcctggtgagtgtggcttcattgctcagctgaatgaaggacgccatctcaagaagcgaccaactgagttccgcgttgataaggtgctccagcctttcgatggaagcaaattcaacttcaccaaagttggtcaggaagaggtgctctttcagtttgaagcaagcgtggatggtgaagctcagttctttcccaatgctccaattgatgttgagagttcccccagtgttgttgccatcaatgtcagtcctattgaatatggtcatgtgctgttgatccctcgcgtcctggagtgcctgccacaaaggattgatcgtgcaagctttttgcttgctttatacatggcttcagaagctggaaacccttatttccgactaggttacaacagcctgggggcatttgccaccatcaatcacctgcatttccaggcatactatttggctgtgcccttccctgttgagaaggcttccactcgaaacataacagccctggacagtggggtgaatgtatctgaacttgtcaattatccagtcagaggtcttgtttatgaaggtggaaataccatacaagacttggccaatgcagtttcggactcatgcatctgccttcaagacaacaatataccttacaacgtcctcatatctgattgtggaaagcgcattttcctcttcccacagtgctatgcggagaagcaagctctcggagaagtcgatgcggaggttctggacacgcaagtgaacccggcagtgtgggaaatcagtgggcatatggtcctgaagaggaagaaagactatgaggaggcatctgaggagaatgcatggaggctcctagcggaggtttccctatctgaggagaggttccgtgaagtgagcgacctgatctttgaagcaatctcttgcagcattgatgacgttgctccaagctcgcttgatgagcccaatgctgaacctctggcggactttgccgccccctgccccaatatggtgcgcgggaagaaggattgcctggttctgcagtgagaagcgatcctttcagtatttgcgatcctttcagtatttgcattgtgggactctgtgtgtgtgtgtgtgcgcgtggaatttccagctgctatggcattgccagcaactgcagaataagcgaacttggtttcgttgctctagcgtcggtgcgtcgtgtagtgtctatgtggaatgtgtcgtctgtgttcacctaaattctgttgctgtatattactgtattggtacgcttctcaggtataccatcaggttactcttgagagcatgatactcttttaagtcttagccaatatttttagttcagatcgatgtttatcgccttcaaataactgctagtcttcttcaggtagtcttctccctctagcctgaagaagactactttttaggggtgttttggcaagttttttcaaagagctttcggtcaaatgttgatcttaatttgaccttgggaaagttaaaagctcaagtaggggtgtcaaa

SEQ ID NO: 146 - protein, 439 aa, Arabidopsis thaliana
MMLKIKRVPTVVSNYQKEEVEGGRVGGCGRNCLNKCCVQGAKFPLYAFKKEKEVPGDRALLGHVEREPPVAFLDSLLLGEWEDRMQRGLFRYDVTACETKVIPGECGFIAQLNEGRHLKKRPTEFRVDKVLQPFDGSKFNFTKVGQEEVLFQFEASVDGEAQFFPNAPIDVESSPSVVAINVSPIEYGHVLLIPRVLECLPQRIDRASFLLALYMASEAGNPYFRLGYNSLGAFATINHLHFQAYYLAVPFPVEKASTRNITALDSGVNVSELVNYPVRGLVYEGGNTIQDLANAVSDSCICLQDNNIPYNVLISDCGKRIFLFPQCYAEKQALGEVDAEVLDTQVNPAVWEISGHMVLKRKKDYEEASEENAWRLLAEVSLSEERFREVSDLIFEAISCSIDDVAPSSLDEPNAEPLADFAAPCPNMVRGKKDCLVLQ

SEQ ID NO: 147 - DNA, 662 bp, Arabidopsis thaliana
  source 1..662: mol_type = genomic DNA
cgggtcgggtcggttcgtttcgggtcggttcgacccgtttttgctcccgccggtcgtccgcgagtcaatctctctttctatctataaaagcgagccaaccaaccttctcctccacccagccaaccaaacatccatcgtcgcccgcatcgcctgcaaaaaaaatctctccttttacctccgccatcgcccgcttcaaatcctaggaaaatccgaacccaagcttctcgattgttcggtcggtcggtcggtcgaaggaaatcgcggcgagtgttgtgtttgtcggggggtaaaacccggggcagacgagttgaggacatcacggctatacacggcgtagcccggccactcatccatgcccgctccgttcgacgccgcaagggttgcctgatcgccgcctcctgcgtcggcaacccttcgccgcacggcggacgaggcgccctcccctccgaaggcggcagcccctccgacctcctcttcctcgccggcggcggtccctcctccctcctctactgctagattattctttcctataggagtcttcgttttcgtccctagctacgacgtattccgccgcgtgtaacgagcaagtcgctgttcttttttttttttagatctcgttttttgtgtttttttcctcgtacttaaaaaaccggggagggaag

SEQ ID NO: 148 - protein, 121 aa, Arabidopsis thaliana
LQKKSLLLPPPSPASNPRKIRTQASRLFGRSVGRRKSRRVLCLSGGKTRGRRVEDITAIHGVARPLIHARSVRRRKGCLIAASCVGNPSPHGGRGALPSEGGSPSDLLFLAGGGPSSLLYC

SEQ ID NO: 149 - DNA, 3610 bp, Miscanthus sinensis
  source 1..3610: mol_type = genomic DNA
ccaccccgcctcctccgcccctcccaagccaaacccggcgcagcgcacaacccccaacgcaccgcaccgccgccgctcgccggagcagaagacaggagaaccccggggcgcaggcctcggcagcttccgcgaccccaccgctgcgatcccgcaacagcctgcccccctcccctcccctctgggcgtggccgagttgtaggcatcacggctagcgttgcagccccccggcgcggccccccagctgcgcaggtggagcccacggggaccatcgcctccgccgtcgcggccagccccgccccgcacggcggccgcggggcgctgccctcggccggaggaagcccgtccgatctcctcttcctcgccggcggcggtcgcctctgagccgcagccggtcctttctctcacccccttcctttcctagcgaatagtcccttagctaacccccgaccccgacgagttctcaccccgagagtgaccgagccaccggcgagagtaaccgacccaccggcatggagatgaagctcaccatcaagagggtgcccaccgtcgtctccaactaccaggacgacgccgacaagccgcgcgccggctgcgggcggaactgcctcggcgactgctgcctgcctggtgagccacctaccccgccgctcgcaggctttcgctttgttttacggtcaatctgacgtcgttttcgtccgcgcaatccgattgaaaatcttagaattattacgatcttggatcctgtcccattttaaattgtgccccgaagattcgtgcatgattcgttcagaaaattcccaaactttgtacagtaaaaggtgtcgtttgtgctccagaaagttgggggcctttgcacctaaacaatcgcaacagggttttcctttttttatctgcactatttttacccatccctccggttaaaaattgttacggccagtggttttggggaacaactggctgttgttattaccccgcctcgaattgggttggattaaataaggatataaataaacaaacatcttaggcaactgtagatatttatcataattctcagccggaaccccaggccatcataactggccttttgtagtattatgtgcctattgtatgcctcacttgaaaatatggtttgttttataactcagtagaaacctcactgagacatcagctctgcatgtacaaaaaactagtattatttgcttcacctgacatgaaagtagcaaattatgctgcacttttatttgaccgacatttgcatctttctttgagcagcttccaagcttcccctctacgcgttcaagttggatccggcaaagcacttgcaggaggaagctgcttcaaccaagttgcttgttgatatcctccttagtgaggtaaataaaagcttgtggcttttgttggacttcaactgtgctagttttaggatgtgatatattaggaatcaaattatctacatctagtctagtaagaaatatgcatatctcttaagaagttggggaaacatatttggaccattattgtattgaggagtggaaaacctgccaggcacaaaccaaacgagccctaagtccacttgagcagtagcaaacgcacgcttgtatggcttgtaagatgatagttgctgcaagaacagtgtctaatatcttcaaatatgggccattcttattttgattaatatataccttacaatggcactagaattggcactgtgaatctaaatgtcgaactctggtttcgaatgatatatggacagttttactttttgttctgtaccacattctataaaagtaggaaatactttattgtgaaagtttgctgaaataggtggataatgaagtctacttctaatatttatcttttcaggattcagattctgagtttagtattttgctttgtattctggcagtgggaggacaggatggcccgaggcttattccgctatgatgtcactgcttgtgagaccaaggtcatccctggcaaccttgggtttgttgcacaactcaatgaagggcgccaccttaagaagcgccctactgagtttcgtgtggaccgtgtgctccagccatttgaccctgccaagttcaatttcaccaaagttggccaggaagaggttctcttccaatttgagaatggcgatggtgatgacagctatttcctgaatgacgctccaatcattgctgttgatcgtgccccaaatgttgttgctatcaatgtacatctctcattcattcacactgacaatccaacttttattctatctcgcctattcttgtttccaatgactgtttgcgatggtttgcaggtgagcccaattgaatatggacatgtgcttctcattcctcgtgttctggaccacctgcctcagaagattgatccagagagcttcttgcttgcactgcaaatggcagctgaggcagctagcccgtacttcaggcttggttacaatagcttgggtgcctttgccaccatcaatcacctccactttcaggtattaaacttctgatggtatctaaatttacttctcacaacaaaatcctaaccaaataggagcagtattgtgaacatagtgttattgtggtaactcctttttttttgaaaatgttgtttagtactgactactgaatgataatttcttgttttctttacaggcatactacttgtcagtgcctttccctgtcgagaaggcagctacccacaagatccctctttctgaggacacaatgaagaatggagtgaccgtgtccaagctgataaattatcctgtgagaggcctggtatttgagggagggaacacactggaggacctggcaaatgttgtttccaatgcttgcatttggctgcaggacaacaatgtgccttacaacgtgctcatttctgactgtggcaagaggatcttcctgttcccccaggtaaaatcattacacaatttcgtagctttgtcaaatctggaccgaattgctggaaatctctttatcatgttctaacaaacttctatctgttcaataatgcagtgctatgcagagaagcaggctctgggcgaagtgagccaggagctgctggacactcaggtgaacccagctgtgtgggagatcagtggccacatcgtgctgaagcgcaggagtgactatgaggaggcatctgaaacctccgcgtggaagctcctcgccgaggtctccctctctgaggagcgctttgaggaagtgaaggcgtacatcttcagcgctgctggcctggttcagtctgacgaggaagaggaggaagccagcgagggcgagggcaccaccacctttgctcctgtcccggttacccctctgcctgtcgctgaaggctgcatcgtccgccagtgagtctgtggactggctgttgtgtcttcctggtcgccataactgtgctggcaataaagctctgttggtactgtataatgcttgcgagcgctgtgctctgctgctactttgctctcgtttgtgtcctgctgctctcgtggtgaactcggtaattcagtgctctgcaatattaagcagaggctattgtattgtttgggtcgtttgttaattattattactagaactaaatgaaatgtgataaacattactagtaaccgctgtaacac

SEQ ID NO: 150 - DNA, 509 bp, Miscanthus sinensis
  source 1..509: mol_type = genomic DNA
ccaccccgcctcctccgcccctcccaagccaaacccggcgcagcgcacaacccccaacgcaccgcaccgccgccgctcgccggagcagaagacaggagaaccccggggcgcaggcctcggcagcttccgcgaccccaccgctgcgatcccgcaacagcctgcccccctcccctcccctctgggcgtggccgagttgtaggcatcacggctagcgttgcagccccccggcgcggccccccagctgcgcaggtggagcccacggggaccatcgcctccgccgtcgcggccagccccgccccgcacggcggccgcggggcgctgccctcggccggaggaagcccgtccgatctcctcttcctcgccggcggcggtcgcctctgagccgcagccggtcctttctctcacccccttcctttcctagcgaatagtcccttagctaacccccgaccccgacgagttctcaccccgagagtgaccgagccaccggcgagagtaaccgacccaccggc

SEQ ID NO: 151 - protein, 435 aa, Miscanthus sinensis
MEMKLTIKRVPTVVSNYQDDADKPRAGCGRNCLGDCCLPASKLPLYAFKLDPAKHLQEEAASTKLLVDILLSEWEDRMARGLFRYDVTACETKVIPGNLGFVAQLNEGRHLKKRPTEFRVDRVLQPFDPAKFNFTKVGQEEVLFQFENGDGDDSYFLNDAPIIAVDRAPNVVAINVSPIEYGHVLLIPRVLDHLPQKIDPESFLLALQMAAEAASPYFRLGYNSLGAFATINHLHFQAYYLSVPFPVEKAATHKIPLSEDTMKNGVTVSKLINYPVRGLVFEGGNTLEDLANVVSNACIWLQDNNVPYNVLISDCGKRIFLFPQCYAEKQALGEVSQELLDTQVNPAVWEISGHIVLKRRSDYEEASETSAWKLLAEVSLSEERFEEVKAYIFSAAGLVQSDEEEEEASEGEGTTTFAPVPVTPLPVAEGCIVRQ

SEQ ID NO: 152 - protein, 68 aa, Miscanthus sinensis
MQPPGAAPQLRRWSPRGPSPPPSRPAPPRTAAAGRCPRPEEARPISSSSPAAVASEPQPVLSLTPFLS

SEQ ID NO: 153 - DNA, 3601 bp, Saccharum officinarum
  source 1..3601: mol_type = genomic DNA
tttctctcttcagagttcagaccgaaatctcggaggaaacttgctttgagtgagagattccaatctttggggcgccattgccgcctgctttctgtgctaggaaaggtaagggtgggcaaaatacccgatacccaattcccgtacccgaattacccgaacccagacccgaagtaccggatcacaatttcggatagtgatcataaaaacccaaaattagttcgggtaattcgggtaatgtttcccggtacccgaactacccaaattacccgaaatatctcaaatatttgctcttctattcctaatatgaagacatgaagtatttcaatgccatataaatatattatttggtatgaattaatgtagatttatgctaacttgataatttatttattggtaattgatatgaagacatgatgtgatttatatgactattgtttattgctttaaaatggtacttgaattcttccaaaatggttggttttgtcaatttcgggtatatcgggtaaaacccgaacctgaacccgaattatcgggtacccgaaatttcgggtaatactttttcggggcaaaaatcgggtaacaatttttaatacccgaatttccagctacccgaattacccgacccgaatttttcgggtaacccgaatgcccacccttggaaaggtgcgttcctttccctgttcattgctggctcgctgctcttttcctcccgctcactccgtcgctggctgcatccgaggtcgcttcctagttcctagtggggctgagcagtccgtcaccatcgtctatctctcggatgcggtgaatcagggccccaagtcttgcagagaaagcggcacaacagcgcgggggctgtcggcgactttggggaactccgtcctgcgtatccgtctctgctttccgtcctccatcgaagcatcccctcgggcgagcgccgtccgccggcgaccgcgagtccacgacagtaaccacttacaattgttgcatattaatccctgatttggatcgcgactaatgatcgtgatatcacacaaatggagcgcgcggccgcaccttcctctcatcgtgaccgtcctggcgctcctgctcgcctcccggtttcccttctctcccgttgcctatccacgtggggttgccgtgaatccaccgtcttctccgcccacaatctctcaacaaagccaccaaatccagtcatccagctgctgatttgagggaaggcctcctttgagtgagatccaacccttggaggccatttccacccgcgtcccccgcaataaaagttgggcatttcctatgttagtcgctgctcttcaaacccacctcgttccatcgctgccgctggttgctcctgctcccgcggcgtcgtttcttcggtatgccggcatcattctcttagctgatgtgatatttctgtgcttgtggtcagggaaaagagtggcctactggcctttatgacttgtcaccgacagcacacatggatcaattgaacatgtggtgtggaaaaactggtggtgaaaaatgggaattgatttgtgcatctggggaatggttttgagcctttggattgggacaaacccaaatgctgagctgtgaaacaacccgtctacttcatgcacctattttctacatctttctttttttttcttttgcacaattgttacatacttccatatgaacaattgggcatttcttaagtgacgcttgttcatagcgaccttgtattgaacgcagtgctagtattgactaatgagctgtatggaacttactagctaacacaaactcatatattgcaggcgctgtctgattcctgtatattttgacattgctttcagctgaaacagaactaaacatggatggcactagttttgacctgacagagcatgaaggttcaaatttgtcaccatttctacgcaaactttttaatgaggtttttacatgtcttagacacatcatgtctgcattccgattatgatttgtattgaaagaatgctagttaaagcatactaaacatgagcggttattgcagtgggacgatcgcaagatgagaggcttgtttcaccatgacatcagttcctgtgaaacaaaggttttctttttcattatctgtttgggtgatttcatctctcttaattttgcataatcatgtcataggtgctacctggagaacacaattttgtggcaacattaattgaaggacgtgaccaaaagaaacggccaactgagttcacaatgaaccaagtcctccagccatttgtcagtgagaagtttaatttcacaaaagttagcccattggaggtgatctttagattcaatgaaactaagaaagactctgcacaatattttgatggtgttcctggtaccgtttcagcttcttctagtgccatcttgattaatgtcagtgaccccccccccccccctggcaaatttacttaatgccttgctgatagaaatacgacatgatttgattaacatttttgtcatcttccgcgtcacaggtgagtccaattgggtactgccatgtccttttgactcccaagatccaggactgtttaccgcaaaggattgatcaagagagctttttaatagccatgtatgttgcaagagaggcaaggaatcccttcttcagagttggttataacagcctgggcggctttgcaactatcaatcaccttcacttccaggttagtcttcagaatgtctatatgtcatatacttctgggttgcagtgtgacaattgacaagtgttcgtacgttacaatcctctcagcaagtcatatactggcattttgttcatcaggcatactacctgaaagtacagtatccagttgaaaaggcaaccacggagaagctcacaagcctagggaatggcgtgagcatttttcacctggtggattatccagtcagtggctttttgttcgaaggtggtgcgagcctggaagatttgtcggatgtggtctccaaagtgtgcatcttcttgcaagagaacaacagacctttcaatgtcctcatctctgaatccggcaagagagttttcctcctaccacaggtattgctatgcaacctcagttgtttgttaagttctgaactctgaaacaatgttcacaattcatcctttagcaatgtgttggcatttatcttcctcaaaaccgattggttagtgctttgcggagaagcagctccttggaaaggcgagccaagagttcctcgacatgagaatcaatccagcaatctgggagctcagtggccatttggtgctgaagaggaggaaggattacgacgaagcctctgaggcaaacatatgcagatttttggttgaagcagctctttctgaaaaagagtttcaggagttgaaccgatgtgtgctcgactttgtgaccagctctcttggttgtaactaggatatagcttcctagacagtaacatttctgaagtttcagttaacagttgtattggtaactgttactttatgatggtatatcgtgtagtagtatttctgatttcactcatcacttctgcgatgaaagtaatatgtaatatgttttctccgacttcaaattcttaccaaacgcacataatcctgctgctgcagagcaactccagg

SEQ ID NO: 154 - DNA, 2618 bp, Medicago truncatula
  source 1..2618: mol_type = genomic DNA
ggaggaattaataattagtaataaaaaaaaaagtggttttcaaaagtaattgggaagttgagagagagatagagcaataaccagtaaccacccaccaaatggtccatcaatatgagaggcagccactaagaatagagtcaaatcttcaaaaaatggaaaagaagaagaagtagacacaccctctttttcctttctcattcaaatctttgtctctttctctctcactttcctctcttgtgaatgtgtaaatgtaacacacacacacacacacaatactaggaaggggtggttacttagccattcattaacaaacactttgcttaacacaacaaaacacaacacaaaaaaacataaaaacaaaacaccctcctcgtgcttgattctagtacaagtataccaaactcattcacaaccacatttccttaatctcttttcattttctcttatctatataacactttacaatctccacctcattttcttcatcaaaaacaattcatctttcttctttgctatcatcactaaattttcttgagaattcagaaaataaaagagagatttgaatgtttgggttgattccctgtattgacacgttgtagacatcacggcttttcttagagtttatcggtcactttcacatgcccgaactgtacgacgtaagggttgttgcttaactccgactaacccttcgccgcacggtgggcgtggtgctctgccttctgaaggtggtagcccctccgatcttctcttcctcgccggtggtggtgtctttgctccttcttccttctaatttcttgtttttagtttaactttctttagatttttacgactcaaaactataagctaaaactagactttgtaagtaagatttatttgaaggtgcttcttcttcttcttctttcttcttcttgttgttaaaaaaaaaagtatttttgtgtttggtttggtgaaaatgatgctaaaaatcaaaagggttcccaccgttgtttccaattatcaaaaagaggaggttggagaggctgctcctcgtactgtcggaggttgtggtcggaattgtctcaaatcttgttgtattcaagatgcaaagctacctttgtatgcttttaaaaagattgacaaagtaaccgaaaaggatttggctatccatgagtgtcaagaggagcttcctatggcctttttggactcacttgttcttggagagtgggaagatcgcatgcaaagaggactttttcgttatgatgttaccgcctgtgaaaccaaggtgattccgggtgagtgtggtttcattgctcagcttaacgagggccgacacctcaagaagagaccaactgagttcagagttgataaggttcttcagccctttgatgaaaacaaattcaactttactaaagttgggcaagaagaggttctgtttcaatttgaagctagtgaagatggtgaagtccagttttatccaaatgccccaattgatgttgacaattatcccagttttgttgccatcaatgtcagtcctattgagtatggacatgttttgctgattccccgcattttcgagtgtttgcctcaaaggattgatcatgagagcttcttgcttgcacttcacatggcagctgaagctgctaatccatattttcgattgggttacaacagccttggtgcatttgcaactataaaccatcttcacttccaggcttattatttggctatgccctttcctattgagaaggctcccaccaagaaaattgcaacttcaaatggtggtgtgaaggtatctgaattgctgaagtatccggttagaggtcttgtctttgaaggcggcgacacgctcgaagatttatcaaaaattgtttcagatgcttgtatctccctacaaaacaacaacataccttacaatgttcttatttctgactgtggaacacaagtgtttcttttaccacagtgttatgcagagaaacaagctcttggagaagtggatgctgagcttcttgacacacaagtaaatcccgcggtgtgggagattagtggacacatggttttgaagaggaaaaaggattttgacgaggcgtctgaagccaatgcttggaggcttcttgcagaagtttcactctctgaagaaaggtttcaagaagttaatgctattatctttgaagccattgccttaactgaagaattggatgacaatgttcaatgtcttccaaaagatgattctgtcgactcgagcacataccctactgtggtggctggttcacaagaatgtgttgttctacagtaaaataattcagtggttgtgcagtgtttaaaaaatttgctgaattatgtctggttgaaataagtacctctaatggtaacatttgcttaagttctagaattatgatgtgcctaatgtttgtgtcatgcattagtactctattgtagtttttgtcatggcttcaccaagcctgaaataacaacttcatgtatctgtttccttagcaaatcagactgcaccctgttgttaaggataatgtcttctaccttttgtatttctggctccacatttgtaataaataaacgtggcattcttcttttgttgatatattttcaatgcaatttgccattaaaaaatgtgcaatttctta

SEQ ID NO: 155 - DNA, 1320 bp, Medicago truncatula
  source 1..1320: mol_type = genomic DNA
atgatgctaaaaatcaaaagggttcccaccgttgtttccaattatcaaaaagaggaggttggagaggctgctcctcgtactgtcggaggttgtggtcggaattgtctcaaatcttgttgtattcaagatgcaaagctacctttgtatgcttttaaaaagattgacaaagtaaccgaaaaggatttggctatccatgagtgtcaagaggagcttcctatggcctttttggactcacttgttcttggagagtgggaagatcgcatgcaaagaggactttttcgttatgatgttaccgcctgtgaaaccaaggtgattccgggtgagtgtggtttcattgctcagcttaacgagggccgacacctcaagaagagaccaactgagttcagagttgataaggttcttcagccctttgatgaaaacaaattcaactttactaaagttgggcaagaagaggttctgtttcaatttgaagctagtgaagatggtgaagtccagttttatccaaatgccccaattgatgttgacaattatcccagttttgttgccatcaatgtcagtcctattgagtatggacatgttttgctgattccccgcattttcgagtgtttgcctcaaaggattgatcatgagagcttcttgcttgcacttcacatggcagctgaagctgctaatccatattttcgattgggttacaacagccttggtgcatttgcaactataaaccatcttcacttccaggcttattatttggctatgccctttcctattgagaaggctcccaccaagaaaattgcaacttcaaatggtggtgtgaaggtatctgaattgctgaagtatccggttagaggtcttgtctttgaaggcggcgacacgctcgaagatttatcaaaaattgtttcagatgcttgtatctccctacaaaacaacaacataccttacaatgttcttatttctgactgtggaacacaagtgtttcttttaccacagtgttatgcagagaaacaagctcttggagaagtggatgctgagcttcttgacacacaagtaaatcccgcggtgtgggagattagtggacacatggttttgaagaggaaaaaggattttgacgaggcgtctgaagccaatgcttggaggcttcttgcagaagtttcactctctgaagaaaggtttcaagaagttaatgctattatctttgaagccattgccttaactgaagaattggatgacaatgttcaatgtcttccaaaagatgattctgtcgactcgagcacataccctactgtggtggctggttcacaagaatgtgttgttctacagtaa

SEQ ID NO: 156 - protein, 439 aa, Medicago truncatula
MMLKIKRVPTVVSNYQKEEVGEAAPRTVGGCGRNCLKSCCIQDAKLPLYAFKKIDKVTEKDLAIHECQEELPMAFLDSLVLGEWEDRMQRGLFRYDVTACETKVIPGECGFIAQLNEGRHLKKRPTEFRVDKVLQPFDENKFNFTKVGQEEVLFQFEASEDGEVQFYPNAPIDVDNYPSFVAINVSPIEYGHVLLIPRIFECLPQRIDHESFLLALHMAAEAANPYFRLGYNSLGAFATINHLHFQAYYLAMPFPIEKAPTKKIATSNGGVKVSELLKYPVRGLVFEGGDTLEDLSKIVSDACISLQNNNIPYNVLISDCGTQVFLLPQCYAEKQALGEVDAELLDTQVNPAVWEISGHMVLKRKKDFDEASEANAWRLLAEVSLSEERFQEVNAIIFEAIALTEELDDNVQCLPKDDSVDSSTYPTVVAGSQECVVLQ

SEQ ID NO: 157 - protein, 442 aa, Arabidopsis thaliana
MLKIKRVPTVVSNYQKDDGAEDPVGCGRNCLGACCLNGARLPLYACKNLVKSGEKLVISHEAIEPPVAFLESLVLGEWEDRFQRGLFRYDVTACETKVIPGKYGFVAQLNEGRHLKKRPTEFRVDKVLQSFDGSKFNFTKVGQEELLFQFEAGEDAQVQFFPCMPIDPENSPSVVAINVSPIEYGHVLLIPRVLDCLPQRIDHKSLLLAVHMAAEAANPYFRLGYNSLGAFATINHLHFQAYYLAMPFPLEKAPTKKITTTVSGVKISELLSYPVRSLLFEGGSSMQELSDTVSDCCVCLQNNNIPFNILISDCGRQIFLMPQCYAEKQALGEVSPEVLETQVNPAVWEISGHMVLKRKEDYEGASEDNAWRLLAEASLSEERFKEVTALAFEAIGCSNQEEDLEGTIVHQQNSSGNVNQKSNRTHGGPITNGTAAECLVLQ

SEQ ID NO: 158 - protein, 431 aa, Arabidopsis thaliana
MLLKIKRVPTVVSNYQKDETVEEGGCGRNCLSKCCINGARLPLYTCKNLDKSVGENTESPVTFLESLVIGEWEDRFQRGLFRYDVTACETKVIPGKYGFIAQLNEGRHLKKRPTEFRVDKVLQPFDGNKFNFTKVGQEELLFQFKASTNDDDSEIQFLASMPLDADNSPSVVAINVSPIEYGHVLLIPRVLDCLPQRIDHKSLLLALQMAAEADNPYFRLGYNSLGAFATINHLHFQAYYLAMQFPIEKASSLKITTTNNGVKISKLLNYPVRGLLVEGGNTIKDLADTVSDASVCLQNNNIPFNILISDSGKRIFLLPQCYAEKQALGEVSSTLLDTQVNPAVWEMSGHMVLKRKEDYEGASEEKAWRLLAEVSLSEERFREVNTMIFDAIGFSSHEEEEEEELEEQNSMNGGSFTIVHCPSVKEEAVSN

SEQ ID NO: 159 - protein, 437 aa, Solanum lycopersicum
MMLKIKRVPTLVSNFQKDEADEIAARGAGCGRNCLRNCCLPGSKLPLYGFKNLSYGKSVADETKESPIDFLESLVLGEWEDRQQKGLFRYDVTACETKVIPGEYGFVAQLNEGRHLKKRPTEFRVDKVLQPFDGSKFNFTKVGQEELLFQFEASEEDEVQLYPDAPIDPEKSPSVVAINVSPIEYGHVLLIPKVLECLPQRIDRDSFLLALHMAAEAANPYFRLGYNSLGAFATINHLHFQAYFLAVQFPIEKAPTQKITVTDAGVKISEMLHYPVRGLVFEGGNTLEDLADVVSDSCICLQENNIPYNVLISDSGKRIFLLPQCYAEKQALGEVSAELLDTQVNPAVWEISGHMVLKRKEDYEGATEANAWRLLAEVSLSEARFQEVTALIFEAISLSVEENEDGTDGSPEDLDVTPPQPMEEIDGLNTHSTMVPA

SEQ ID NO: 160 - protein, 437 aa, Solanum lycopersicum; identical to SEQ ID NO: 159

SEQ ID NO: 161 - protein, 438 aa, Solanum lycopersicum
MLTVKRVPTLVSNYQEDVLEGNVMGCGRKCLGKCCMPVSVLPLYAFKNDDNEPIENDVQTLPEEECQMSFLNDLLLGLWEERMSQGLFRYDVTTCETKVIPGRCGFIAQLNEGRHLKKRPTEFCIDKVLQPFDENKFNFTKVGQEEVLFRFEPSTDYKAHYFSGMRVNSGISPSIVAINVSPIEYGHVLLIPRVLDCLPQRIDRDSFAIALHFAREVADPFFRVGYNSLGAFATINHLHFQAYYLSVPFPVEKAPIQKILARKGLGGAGVIVSKLLNYPVRGFAFEGGNGSTARDLSDAVVNSCISLQNKNIPFNILIAQCGKKIFLLPQCYAEKQALGVVDQELLDTQVNPAVWEISGHIVLKRTKDYNDASEEYAWKLLSEVSISEERFEEVKGYISEAADLQADEDENINPEKEIPDSPGPQVASHIPPDCLVLQ

SEQ ID NO: 162 - protein, 437 aa, Solanum lycopersicum; identical to SEQ ID NO: 159

SEQ ID NO: 163 - protein, 431 aa, Triticum aestivum
MEMKLTIKRVPTVLSNYQEEGGGGCGRNCLGDCCLPASKLPLYAFKADPKKPAQDDELPTEFFLNSLLLAQWEDRVARGLFRYDVTACETKVIPGELGFVAQLNEGRHLKKRPTEFRVDRVLQPFDSAKFNFTKVGQEEVLFRFENGGGDNSYFLENAPSTEGDHAPSVVAINVSPIEYGHVLLIPRVLDRLPQQIDPESFLLALHMAAEAASPYFRLGYNSLGAFATINHLHFQAYYLSVPFPVEKAPTKKIPLAKCALNSGVKVSKLTNFPVRGLVFERGNTLKDLADVVTNACIWLQENNVPFNVLISDSGRRIFVFPQCYAEKQALGEVSQDLLDTQVNPAVWEISGHIVLKRRTDFKEASEASAWRLLAEVSLSEERFEEVKACIFEATGLTESDEEEESSESPYASSSSVPRASSHMSEGCLVLQ

SEQ ID NO: 164 - protein, 431 aa, Triticum aestivum
MEMKLTIKRVPTVLSNYQEEGGGGCGRNCLGDCCLPVSKLPLYAFKADPKKPAQDDELPTEFFLNSLLLAQWEDRVARGLFRYDVTACETKVIPGELGFVAQLNEGRHLKKRPTEFRVDRVLQPFDSAKFNFTKVGQEEVLFRFGNGGGGSSYFLENAPSTEGDHAPSVVAINVSPIEYGHVLLIPRVLDRLPQQIDPESFLLALHMAAEAASPYFRLGYNSLGAFATINHLHFQAYYLSVPFPVEKAPTKKIPLAKCALNSGVKVSKLTNFPVRGLVFERGNTLKDLADVVTNACIWLQENNVPFNVLISDSGRRIFVFPQCYAEKQALGEVSQDLLDTQVNPAVWEISGHIVLKQRTDFEEASEASAWRLLAEVSLSEERFEEVKACIFEATGLTESDEEEESSESPYASSSSVPQASSHMSEGCLVLQ

SEQ ID NO: 165 - protein, 431 aa, Triticum aestivum
MEMKLTIKRVPTVLSNYQEEGGGGCGRNCLGDCCLPASKLPLYAFKADPKKPAQDDELPTEFFLNSLLLAQWEDRVARGLFRYDVTACETKVIPGELGFVAQLNEGRHLKKRPTEFRVDRVLQPFDSAKFNFTKVGQEEVLFRFEKGGGDSSYFLENAPSTEGDHAPSVVAINVSPIEYGHVLLIPRVLDRLPQQIDPESFLLALHMAAEAASPYFRLGYNSLGAFATINHLHFQAYYLSVPFPVEKAPTKKIPLAKCALNSGVKVSKLTNFPVRGLVFERGNTLKDLADVVTNACIWLQENNVPFNVLISDSGRRIFVFPQCYAEKQALGEVSQDLLDTQVNPAVWEISGHIVLKRRTDFEEASEASAWRLLAEVSLSEERFEEVKACIFEATGLTESDEEEESSESPYASSSSVPRASSHMSEGCLVLQ

SEQ ID NO: 166 - protein, 430 aa, Triticum aestivum
MKLTIKRVPTVVSNYQEDAGERPRGGCGRNCLGHCCLPVSELPVYAFKANPTKLPLQEDAVPTDFFINLLLGQWEDRMTQGLFRYDVTACETKVIPGNLGFVAQLNEGRHLKKRPTEFRVDRVLQPFDSAKFNFTKVGQEEALFQFENSGSDDSYFLRSAAATVADRAPNVVAINVSPIEYGHVLLIPRVLDHLPQRIDQESFLLALHMAAEAARPYFRLGYNSLGAFATINHLHFQAYYLTVPFPVEKAATQRIPLAEGGIKSGVKVSKLMNYPVRGLVFEEGNTLNDLANLVSSACIWLQDNNVPYNVLISDSGRKIFLFPQCYAEKQALGEVSQELLDTQVNPAVWEISGHIVLKRRDDYEEASEASAWRLLAEVSLSEARFEEVKAYIFDAACLVQSQVEEETDDAIYAPVPVGPSAVGEGCLVIP

SEQ ID NO: 167 - protein, 430 aa, Triticum aestivum
MKLTIKRVPTVVSNYQEDAGERPRGGCGRNCLGHCCLPVSELPVYAFKANPTKLPLQEDAVPTDFFINLLLGQWEDRMTQGLFRYDVTACETKVIPGNLGFVAQLNEGRHLKKRPTEFRVDRVLQPFDSAKFNFTKVGQEEVLFQFENSGSDDSYFLRSAAVTVADRAPNVVAINVSPIEYGHVLLIPRVLDHLPQRIDQESFLLALHMAAEAASPYFRLGYNSLGAFATINHLHFQAYYLTVPFPVEKAATQRIPLAEGGIKSGVKVSKLMNYPVRGLVFEKGYTLNDLANVVSSACIWLQDNNVPYNVLISDSGRKIFLFPQCYAEKQALGDVSQELLDTQVNPAVWEISGHIVLKRRDDYEEASEASAWRLLAVVSLSEARFEEVKAYIFDAACLVQSQVEEEIDDAIYAPVPVGPSAVGEGCLVIP

SEQ ID NO: 168 - protein, 430 aa, Triticum aestivum
MKLTIKRVPTVVSNYQEDAGERPRGGCGRNCLGHCCLPVSELPVYAFKANPTKLPLQEDAVPTDFFINLLLGQWEDRMTQGLFRYDVTACETKVIPGNLGFVAQLNEGRHLKKRPTEFRVDRVLQPFDSAKFNFTKVGQEEVLFQFENSGSDDSYFLRSAAVTVADRAPNVVAINVSPIEYGHVLLIPRVLDHVPQRIDQESFLLALHMAAEAASPYFRLGYNSLGAFATINHLHFQAYYLTVPFPVEKAATQRIPLAEGGIKSGVKVSKLMNYPVRGLVFEEGNTLNDLANVVSSACIWLQDNNVPCNVLISDSGRKIFLFPQCYAEKQALGEVSQELLDTQVNPAVWEISGHIVLKRRDDYEEASEASAWRLLAEVSLSEARFEEVKAYIFDAACLVQSQAEEETDEAIYAPVLVGPSAVGEGCLVIP

SEQ ID NO: 169 - protein, 430 aa, Triticum aestivum; identical to SEQ ID NO: 166

SEQ ID NO: 170 - protein, 430 aa, Triticum aestivum; identical to SEQ ID NO: 167

SEQ ID NO: 171 - protein, 430 aa, Triticum aestivum; identical to SEQ ID NO: 167

SEQ ID NO: 172 - protein, 430 aa, Triticum aestivum; identical to SEQ ID NO: 167

SEQ ID NO: 173 - protein, 430 aa, Triticum aestivum; identical to SEQ ID NO: 167

SEQ ID NO: 174 - protein, 430 aa, Triticum aestivum; identical to SEQ ID NO: 168

SEQ ID NO: 175 - protein, 431 aa, Triticum aestivum; identical to SEQ ID NO: 163

SEQ ID NO: 176 - protein, 431 aa, Triticum aestivum; identical to SEQ ID NO: 164

SEQ ID NO: 177 - protein, 431 aa, Triticum aestivum; identical to SEQ ID NO: 165

SEQ ID NO: 178 - protein, 369 aa, Triticum aestivum
MVSATKLEGGYCFHSKNSPLDQPEGVKIHLYRLGIEQREHDALKSFACAGEGSPSLLDTIILSQWENFAQKGQLGYDVTTCELKVIEGQRDFVIQMNDKWNSFSLKKYDKFGHPFGCLKPNSARSYEELLLCIAEGENNEPEVVPSITPPNNGVLLIANAYPVEYGHIFLVPNATNQLSSFWDKRMFGLITKIASEVNSAAFRVFFDDGTSIVPKHMFFQACYFANPLPVESSSTVALYDGATRSGICVSEIVDYPLKALVFTSTNVNALVDVVSEACLALHENNTVHSLMMSNNGRNVFLFPQVKNLVTGCCLSAWECCGYFVYHTKVDFDRASETGISNRMASFSFQDGDFEDLKKLCCAIADGLVT

SEQ ID NO: 179 - protein, 369 aa, Triticum aestivum; identical to SEQ ID NO: 178

SEQ ID NO: 180 - protein, 369 aa, Triticum aestivum
MVSATKLEGGYCFHSKNSPLDQPEGVKIHLYRLGIEQCEHDTLKSFACAGEGSPSLLDTIILSQWENFAQKGQLGYDVTTCELKVIEGQRDFVIQMNDKWNSFSLMKYDKFGHPFGCLKPNSARSYEELLLCIAEGENNEPEVVPSITPPNNGVLLIANAYPVEYGHIFLVPNATNQLSSFWDKRMFGLITKIASEVNSAAFRVFFDDGTSIVPKHMFFQACYFANPLPVESSSTVALYDGATRSGICVSEIVDYPLKALVFTSTNVNTLVDVVSEACLALHENNTVHSLMMSNNGRNVFLFPQVKNLVTGCCLSAWECCGYFVYHTKVDFDRASETGISNRMASFSFQDGDFEDLKKLCCAIADSLVT

SEQ ID NO: 181 - protein, 369 aa, Triticum aestivum; identical to SEQ ID NO: 180

SEQ ID NO: 182 - protein, 369 aa, Triticum aestivum
MVSATKLEGGYCFHSKNSPLDQPEGVKIHLYRLGIEQREHDALKSFAYAGQGSPSLLDTLILSQWENFAQKGHMGYDVTACELKVIEGQRDFVIQMNDKWNTFPLKKHDKFGHPFGCLKPNSAKSYEELLLCIAEGEDTEPEVVPSTTPPNNGVLLIANAYPVEYGHIFLVPNSTNQLSSFWDKRMFGLITKIASEVNSTAFRVFFDDGTSIVPKHMFFQACYFANPLPVESASTVALYDGATRSGICVSEIVDYPLKALVFTSTNVNALVDVVSEACLALHEKNTVHSLMMSNNGRNVFLFPQVKNLVTGCYLSAWECCGYFVYQTKVDFDRASETGISNRMASFSFQDGEFEDLKKLCCAIADDLVT

SEQ ID NO: 183 - protein, 369 aa, Triticum aestivum; identical to SEQ ID NO: 182

SEQ ID NO: 184 - protein, 369 aa, Triticum aestivum; identical to SEQ ID NO: 182

SEQ ID NO: 185 - protein, 360 aa, Triticum aestivum
MKLCMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 186 - protein, 360 aa, Triticum aestivum; identical to SEQ ID NO: 185

SEQ ID NO: 187 - protein, 360 aa, Triticum aestivum; identical to SEQ ID NO: 185

SEQ ID NO: 188 - protein, 361 aa, Triticum aestivum
MKLSGMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 189 - protein, 361 aa, Triticum aestivum; identical to SEQ ID NO: 188

SEQ ID NO: 190 - protein, 365 aa, Triticum aestivum
MKLFETKAGMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 191 - protein, 365 aa, Triticum aestivum; identical to SEQ ID NO: 190

SEQ ID NO: 192 - protein, 365 aa, Triticum aestivum; identical to SEQ ID NO: 190

SEQ ID NO: 193 - protein, 388 aa, Triticum aestivum
MRSKRLLASSPTNKCLMFLRGLTLLPVETKAGMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 194 - protein, 388 aa, Triticum aestivum; identical to SEQ ID NO: 193

SEQ ID NO: 195 - protein, 392 aa, Triticum aestivum
MPVISPLPFSLAPTPAAPAASRARWNSFLHHWRLRGMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 196 - protein, 393 aa, Triticum aestivum
MPVISPLPFSLAPTPAAPAASRARWNSFLHHWRLRAGMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 197 - protein, 397 aa, Triticum aestivum
MPVISPLPFSLAPTPAAPAASRARWNSFLHHWRLRVETKAGMCSNGFDSTNCLRMEEVEPNLSPFLHKLFKEWDDRNARGLFHHDITTSEAKVLPGEHNFVATLIEGRDQKKRPTEFGMNQVLQPFHSEKFNFTKVKPEEVIFRFQETENDSAQYFDGTPPTVSASPSCILINVSPIGYCHVLLTPQIQECLPQRVDQESFLLAMYVAREARNPFFRVGYNSLGAFATINHLHFQAYYLKVQYPVENAPTERITVVRNGVSISQLVRYPVSGFVFEGGASLEDLSQAVSDACIFLQENNRPFNVLISESGKRVFLLLQCYAEKQASGKASQEFLDMRINPAVWELSGHLVLKRRKDYDEASEATLCRFLVEASLSEAEFQELKRCLLEFLATASPEK

SEQ ID NO: 198 - protein, 392 aa, Triticum aestivum
MPVISPLPFSLLPAPAAPAASRARWNSFPHHRLLRGMCSNGFDSTNCLRMEEVEPNLSPFLHKLFKEWDDCKARGLFHHNITTCETKVLPGEHNFVATLIEGRDQKKRPTEFGMNQVLQPFHSDKFNFTKVKPEEVIFRFQETENDSAQYFDGAPPTVSASPSSILINVSPIGYCHVLLIPQIQECLPQRVDRESFLLAMYVAREAKNPFFRVGYNSLGAFATINHLHFQAYYLKVQYPVEKAPTEKLTVVGNGVSISQLVRYPVSGFAFEGGASLEDLSQVVSDACIFLQENNRPFNVLISESGKRLFLLLQCYAEKQASGKASQEFLDMRINPAVWELSGHLVLKRRKDYDEASAATLCGFLVEASLSEAEFQELKKCVLEFLASASPEK

SEQ ID NO: 199 - protein, 393 aa, Triticum aestivum
MPVISPLPFSLLPAPAAPAASRARWNSFPHHRLLRAGMCSNGFDSTNCLRMEEVEPNLSPFLHKLFKEWDDCKARGLFHHNITTCETKVLPGEHNFVATLIEGRDQKKRPTEFGMNQVLQPFHSDKFNFTKVKPEEVIFRFQETENDSAQYFDGAPPTVSASPSSILINVSPIGYCHVLLIPQIQECLPQRVDRESFLLAMYVAREAKNPFFRVGYNSLGAFATINHLHFQAYYLKVQYPVEKAPTEKLTVVGNGVSISQLVRYPVSGFAFEGGASLEDLSQVVSDACIFLQENNRPFNVLISESGKRLFLLLQCYAEKQASGKASQEFLDMRINPAVWELSGHLVLKRRKDYDEASAATLCGFLVEASLSEAEFQELKKCVLEFLASASPEK

SEQ ID NO: 200 - protein, 397 aa, Triticum aestivum
MPVISPLPFSLLPAPAAPAASRARWNSFPHHRLLRVETKAGMCSNGFDSTNCLRMEEVEPNLSPFLHKLFKEWDDCKARGLFHHNITTCETKVLPGEHNFVATLIEGRDQKKRPTEFGMNQVLQPFHSDKFNFTKVKPEEVIFRFQETENDSAQYFDGAPPTVSASPSSILINVSPIGYCHVLLIPQIQECLPQRVDRESFLLAMYVAREAKNPFFRVGYNSLGAFATINHLHFQAYYLKVQYPVEKAPTEKLTVVGNGVSISQLVRYPVSGFAFEGGASLEDLSQVVSDACIFLQENNRPFNVLISESGKRLFLLLQCYAEKQASGKASQEFLDMRINPAVWELSGHLVLKRRKDYDEASAATLCGFLVEASLSEAEFQELKKCVLEFLASASPEK

SEQ ID NO: 201 - protein, 436 aa, Glycine max
MMLRIKRVPTVVSNYQKDDAADSPRPVGGCGRNCLKACCLPDAKLPLYAFKKANGKDLALHACAGEPPLTFLDSLLLGEWEDRMQRGLFRYDVTACETKVIPGEYGFVAQLNEGRHLKKRPTEFRVDKVLQPFDENKFNFTKVGQEEVLFQFEASEDGQVQFFPNAPVDVDNSPSFVAINVSPIEYGHVLLIPRIFECLPQRIDRESFLLALHMAVEADNPYFRLGYNSLGAFATINHLHFQAYYLALPFPIEKAPTKKIASLNGGVKISELLKYPVRGLVFEGGDTLEDLSNVVSDACICLQNNNIPFNVLISDCGKQVYLLPQCYAEKQALGEVDAELLDTQVNPAVWEISGHMVLKRKKDYDEASEGNAWRLLAEVSLSQERFQEVNDLIFEAIGCGELEDVNVESVKEVDAVSTSEHPTVVVAGSQECVILH

SEQ ID NO: 202 - protein, 436 aa, Glycine max; identical to SEQ ID NO: 201

SEQ ID NO: 203 - protein, 439 aa, Glycine max
MMLKIKRVPTVVSNFQKDDAADSPRPVGGCGRNCLKACCIPDAKLPLYGFKKANIGKDLALHACAGEPPLAFLDSLLLGEWEDRMQRGLFRYDVTACETKVIPGEYGFVAQLNEGRHLKKRPTEFRVDKVLQPFDENKFNFTKVGQEEVLFQFEASEDGQVQFFPNAPVDVDNSPSFVAINVSPIEYGHVLLIPRIFECLPQRIDRESFLLALQMAVEADNPYFRLGYNSLGAFATINHLHFQAYYLALPFPIEKAPTKRIASVNGGVKISELLKYPVRGFVFEGGEMLEDLSNVVSDACICLQNNNIPFNVLISDCGKQVYLLPQCYAEKQALGEVNAELLDTQVNPAVWEISGHMVLKRRKDYDEASEGNAWRLLAEVSLSQERFQEVNDLVFEAIGCGELEDVNVQSVKKEVDAVSTSEHPTVVVVAGSQECVVLQ

SEQ ID NO: 204 - protein, 346 aa, Glycine max
MLSIKRVPTVVSNYQKEETGEAAAAGGCGRNCLKSCCIQDARLPLYAFKRGEKVGGKELPLLGCKKHPVDFLDSLVLGEWEDRMQRGLFRYDVTTCETKVIPGKYGFIAQLNEGRHLKKRPTEFRVDKVLQPFDENKFNFTKVGQEEVLFQLEASNDGEAQFFPNVPIDVENSPSFVAINVSPIEYGHVLLIPQIFECLPQRIDHASFLLALQMAAEARNPYFRLGYNSLGAFATINHLHFQAYYLALPFPIEKAPTKKIAKLSGGVKISKLLNYPVRGLVFEGGHSLEGLANVVSEACICLQHNNIPYNILISDCGRQIFLLPQNFFVGFGSYNFVYNGTSCAVH

SEQ ID NO: 205 - protein, 436 aa, Glycine max; identical to SEQ ID NO: 201

SEQ ID NO: 206 - protein, 436 aa, Glycine max
MLSIKRVPTVVSNYQKEETGEAAAGGCGRNCLKSCCIQGGARLPLYAFKKVDKVCGNELPLLGCKEQHPVAFLDSLVLGEWEDRMQRGLFRYDVTACETKVIPGEYGFIAQLNEGRHLKKRPTEFRVDKVLQPFDENKFNFTKVGQEEVLFQLEASNDGEVQFFPNAPIDVENSPSFVAINVSPIEYGHVLLIPRIFECLPQRIDHASFLLALQMAVEARNPYFRLGYNSLGAFATINHLHFQAYYLALPFPIEKAPTKKIAKLSGGVIISKLLNYPVRGLVFEGGHTLEDLANAVSEACICLQHNNIPYNVLISDCGRQIFLLPQCYAEKQALGEVSPELLETQVNPAVWEISGHMVLKRKKDYEEASEANAWRLLAEVSLSEERLLEVTALVFQAITSSVEMNVKPQRLEEVDAVSPNAQHAMVAGSHECLVLQ

SEQ ID NO: 207 - protein, 439 aa, Glycine max; identical to SEQ ID NO: 203

SEQ ID NO: 208 - protein, 435 aa, Glycine max
MLSIKRVPTVVSNYQKEETGEAAAGGCGRNCLKSCCIQGARLPLYAFKKVDKVCGNELPLLGCKEQHPVAFLDSLVLGEWEDRMQRGLFRYDVTACETKVIPGEYGFIAQLNEGRHLKKRPTEFRVDKVLQPFDENKFNFTKVGQEEVLFQLEASNDGEVQFFPNAPIDVENSPSFVAINVSPIEYGHVLLIPRIFECLPQRIDHASFLLALQMAVEARNPYFRLGYNSLGAFATINHLHFQAYYLALPFPIEKAPTKKIAKLSGGVIISKLLNYPVRGLVFEGGHTLEDLANAVSEACICLQHNNIPYNVLISDCGRQIFLLPQCYAEKQALGEVSPELLETQVNPAVWEISGHMVLKRKKDYEEASEANAWRLLAEVSLSEERLLEVTALVFQAITSSVEMNVKPQRLEEVDAVSPNAQHAMVAGSHECLVLQ

SEQ ID NO: 209 - protein, 438 aa, Solanum tuberosum
MLTIKRVPTLVSNYQEDVLEGNVMGCGRKCLGKCCMPVSVLPLYAFKNDDNEPIENDIQTLPEEECQMSFLNNLLLGLWEERMSQGLFRYDVTTCETKVIPGRCGFIAQLNEGRHLKKRPTEFCIDKVLQPFDENKFNFTKVGQEEVLFRFEPSTDYKPHYFSGMRVNGGISPSIVAINVSPIEYGHVLLIPRVLDCLPQRIDRDSFAIALHFAREVADPFFRVGYNSLGAFATINHLHFQAYYLSVPFPVEKAPIQKILARKGLGGAGVIVSKLLNYPVRGFAFEGGNGSTARDLSDAVVNSCISLQNKNIPFNILIAQCGKKIFLLPQCYAEKQALGVVDQELLDTQVNPAVWEISGHIVLKRTKDYNDASEEYAWKLLSEVSLSEERFEEVKGYISEADGLQAEEDKNINPEKEIPDSPGPQVASHMPPDCLVLQ

SEQ ID NO: 210 - protein, 438 aa, Solanum tuberosum
MMLKIKRVPTLVSNFQKDEADEIGARGAGCGRNCLRNCCLPGSKLPLYGFKNLSYGKSVAADETKESPIDFLESLVLGEWEDRQQKGLFRYDVTACETKVIPGEYGFVAQLNEGRHLKKRPTEFRVDKVLQPFDGSKFNFTKVGQEELLFQFEASEEDEVQLYPNAPIDPEKSPSVIAINVSPIEYGHVLLIPKVLECLPQRIDRDSFLLALHMAAEAANPYFRLGYNSLGAFATINHLHFQAYFLAVQFPIEKAPTQKITVTDTGVKISEMLNYPVRGLVFEGGNTLEDLANVVSDSCICLQENNIPYNVLISDSGKRIFILPQCYAEKQALGEVSAELLDTQVNPAVWEISGHMVLKRKEDYEGATEANAWRLLAEVSLSEARFQEVTALIFEAISLSVEENENANDGSPEDLDVTPPQPMEEIDGLNTHSTMVPA

SEQ ID NO: 211 - protein, 358 aa, Zea mays
MLSAATKLTMDGTSFDLTEHEGSNLSPFLLKLFNEWDDRKTRGLFHHDISSCETKVLPGVHNFVATLIEGRDQKKRPTEFTMNQVLQPFVSEKFNFTKVSPLEVIFRFNDTEKDSAQYFDGVPDTVSASSSAILINVSPIGYCHVLLTPKIQDCLPQRIDQESFLIAMYVAREARTPFFRVGYNSLGGFATINHLHFQAYYLKVQYPVEKAITEKLKTLGNSVSIFHLVDYPVNGFVFEGGASLEDLSDVVSKVCIFLQENNRPFNVLISESGKRIFLLPQCYAEKQLLGKASQEFLDMRINPAIWELSGHLVLKRRKDYEEASEANICRFLVEAALSETEFQELNLCVLDFLTSSLG

SEQ ID NO: 212 - protein, 435 aa, Zea mays
MEMKLTIRRVPTVVSNYQEDADKPRAGCGRNCLGDCCLPASKLPLYAFKLSPAKGSQEDAASTKLLVDILLSEWEDRMTRGLFRYDVTACETKVIPGNLGFVAQLNEGRHLKKRPTEFRVDRVLQPFDPAKFNFTKVGQEEVLFQFENSGGDDSYFLNNAPIIAVDRAPNVIAINVSPIEYGHVLLIPRVLDRLPQRIDPESFLLALQMAAEGGSPYFRLGYNSLGAFATINHLHFQAYYLSVPFPVEKAATHKIPLSEDTKKNGVTVSKLINYPVRGLVFEGGNTLDDLATVVSNACIWLQDNNVPYNVLISDCGKRVFLFPQCYAEKQALGEVSQELLDTQVNPAVWEISGHMVLKRRMDYEEASETSAWKLLAEVSLSEERFEEVKAYIFSAAGLVQYDEEEEEASDGEGPTAYVPVKVTPLPVAEGCLVLP

SEQ ID NO: 213 - protein, 358 aa, Zea mays
MTRGLFRYDVTACETKVIPGNLGFVAQLNEGRHLKKRPTEFRVDRVLQPFDPAKFNFTKVGQEEVLFQFENSGGDDSYFLNNAPIIAVDRAPNVIAINVSPIEYGHVLLIPRVLDRLPQRIDPESFLLALQMAAEGGSPYFRLGYNSLGAFATINHLHFQAYYLSVPFPVEKAATHKIPLSEDTKKNGVTVSKLINYPVRGLVFEGGNTLDDLATVVSNACIWLQDNNVPYNVLISDCGKRVFLFPQCYAEKQALGEVSQELLDTQVNPAVWEISGHMVLKRRMDYEEASETSAWKLLAEVSLSEERFEEVKAYIFSAAGLVQYDEEEEEASDGEGPTAYVPVKVTPLPVAEGCLVLP

SEQ ID NO: 214 - protein, 422 aa, Zea mays
MLLQPSYLLIFSLVRACLGPRDLEGIEEAKILYAFKLTPAKGSQEDAASTKLLVDILLSEWEDRMTRGLFRYDVTACETKVIPGNLGFVAQLNEGRHLKKRPTEFRVDRVLQPFDPAKFNFTKVGQEEVLFQFENSGGDDSYFLNNAPIIAVDRAPNVIAINVSPIEYGHVLLIPRVLDRLPQRIDPESFLLALQMAAEGGSPYFRLGYNSLGAFATINHLHFQAYYLSVPFPVEKAATHKIPLSEDTKKNGVTVSKLINYPVRGLVFEGGNTLDDLATVVSNACIWLQDNNVPYNVLISDCGKRVFLFPQCYAEKQALGEVSQELLDTQVNPAVWEISGHMVLKRRMDYEEASETSAWKLLAEVSLSEERFEEVKAYIFSAAGLVQYDEEEEEASDGEGPTAYVPVKVTPLPVAEGCLVLP

SEQ ID NO: 215 - protein, 464 aa, Zea mays
MEMKLTIRRVPTVVSNYQEDADKPRAGCGRNCLGDCCLPASKLPLYAFKLTPAKGSQEDAASTKLLVDILLSEWEDRMTRGLFRYDVTACETKVIPGNLGFVAQLNEGRHLKKRPTEFRVDRVLQPFDPAKFNFTKVGQEEVLFQFENSGGDDSYFLNNAPIIAVDRAPNVIAINVHLSVTLEIQLPFYLFLFPLMFFLQWFLQVSPIEYGHVLLIPRVLDRLPQRIDPESFLLALQMAAEGGSPYFRLGYNSLGAFATINHLHFQAYYLSVPFPVEKAATHKIPLSEDTKKNGVTVSKLINYPVRGLVFEGGNTLDDLATVVSNACIWLQDNNVPYNVLISDCGKRVFLFPQCYAEKQALGEVSQELLDTQVNPAVWEISGHMVLKRRMDYEEASETSAWKLLAEVSLSEERFEEVKAYIFSAAGLVQYDEEEEEASDGEGPTAYVPVKVTPLPVAEGCLVLP

SEQ ID NO: 216 - protein, 349 aa, Zea mays
MDGTSFDLTEHEGSNLSPFLLKHFNEWDDRKTRGLFHHDISSCETKVLPGVHNFVATLIEGRDQKKRPTEFTMNQVLQPFVSEKFNFTKVSPLEVIFRFNDTEKDPAQYFDGVPDTVSASSSAILINVSPIGYCHVLLTPKIQDCLPQRIDQESFLIAMYVAREAKTPFFRVGYNSLGGFATINHLHFQAYYLKVQYPVEKAITEKLKTLGNSVSIFHLVDYPVNGFVFEGGASLEDLSDVVSKVCIFLQENNRSFNVLISESGKRIFLLPQCYAEKQLLGKASQEFLDMRINPAIWELSGHLVLKRRKDYEEASEANICRFLVEAALSETEFQELNRCVLDFLTSSLG

SEQ ID NO: 217 - protein, 390 aa, Zea mays
MSVTSLLAQPIPSLFAPSPAPSRARRNRTSRRRFLAATKLTMDGTSFDLTEHEGSNLSPFLLKLFNEWDDRKTRGLFHHDISSCETKVLPGVHNFVATLIEGRDQKKRPTEFTMNQVLQPFVSEKFNFTKVSPLEVIFRFNDTEKDSAQYFDGVPDTVSASSSAILINVSPIGYCHVLLTPKIQDCLPQRIDQESFLIAMYVAREARTPFFRVGYNSLGGFATINHLHFQAYYLKVQYPVEKAITEKLKTLGNSVSIFHLVDYPVNGFVFEGGASLEDLSDVVSKVCIFLQENNRPFNVLISESGKRIFLLPQCYAEKQLLGKASQEFLDMRINPAIWELSGHLVLKRRKDYEEASEANICRFLVEAALSETEFQELNLCVLDFLTSSLG

SEQ ID NO: 218 - protein, 358 aa, Zea mays; identical to SEQ ID NO: 211

SEQ ID NO: 219 - protein, 435 aa, Zea mays
MEMKLTIRRVPTVVSNYQEDADKPRAGCGRNCLGDCCLPASKLPLYAFKLTPAKGSQEDAASTKLLVDILLSEWEDRMTRGLFRYDVTACETKVIPGNLGFVAQLNEGRHLKKRPTEFRVDRVLQPFDPAKFNFTKVGQEEVLFQFENSGGDDSYFLNNAPIIAVDRAPNVIAINVSPIEYGHVLLIPRVLDRLPQRIDPESFLLALQMAAEGGSPYFRLGYNSLGAFATINHLHFQAYYLSVPFPVEKAATHKIPLSEDTKKNGVTVSKLINYPVRGLVFEGGNTLDDLATVVSNACIWLQDNNVPYNVLISDCGKRVFLFPQCYAEKQALGEVSQELLDTQVNPAVWEISGHMVLKRRMDYEEASETSAWKLLAEVSLSEERFEEVKAYIFSAAGLVQYDEEEEEASDGEGPTAYVPVKVTPLPVAEGCLVLP

SEQ ID NO: 220 - protein, 446 aa, Prunus dulcis
MMLRIKRVPTVVSNYQKDEAEEGARRVGGCGRNCLNQCCIPGAKLPLYAFKKLNKIDGDKELPGCEKREPPVDFLDSLLLGEWEDRMQRGLFRYDVTACETKVIPGQFGFIAQLNEGRHLKKRPTEFRVDKVLQPFDGNKFNFTKVGQEEVLFQFEASEDGEVQFIPSAPIEPENSPSVVAINVSPIEYGHVLLIPRILEHLPQRIDRESFLLALHMAAEAGNPYFRLGYNSLGAFATINHLHFQAYYLAVTFPIEKAPSKKITVSGAGVKVSELLNYPVRGLVFEGGNTLQDLSNTVSDACICLQENNIPYNVLISDCGKRIFLLPQCYAEKQALGEVRAELLDTQVNPAVWEISGHMVLKRKKDYEEASDENAWKLLAEVSLSEERFQEVNALIFEAIASDDNGNANLLEDPEVKPHSREEVDTINTSSHAAMVTGTQQCLVLQ

SEQ ID NO: 221 - protein, 368 aa, Prunus dulcis
MVTVEQLQDDNFLSQCPPPLSLQGIRIPLYRFGSNSVLDNGSVGGISCLIEEEQSVLDSILLAQWEDRMWKGLFRYDVTTSEIKVIGGRRKFIAQFNEGWSRDCMPKLGKNKIRHQKDVFEFSWMERQEELLFCVASGEKLNPELVLSAAVPDCGLLITINSSPVEYGHVFLVPHSSYILHQFLDARSLELVLHVAVEVNNCSFRLFYDYSPSASHIYFQACYLQIPLPVEFMPLDTFFGDGQEGMRISSVTDYPIKTLVFESNHLNMMVEILAEICSCLQEKLIPYNLLISDHGKRIFLFFQLQTSKNFCTLSAWECGGYFVVKARSEFDQATEQALLKQLTTVSLDDEGFQGVKRLCCTIASKFAS

SEQ ID NO: 222 - protein, 518 aa, Prunus dulcis
MMLRIKRVPTVVSNYQKDEAEEGARRVGGCGRNCLNQCCIPGAKLPLYAFKKLNKIDGDKELPGSEKRGPPVDFLDSLLLGEWEDRMQRGLFRYDVTACETKVIPGQFGFIAQLNEGRHLKKRPTEFRVDKVLQPFDGNKFNFTKVGQEEVLFQFEASEDGEVQFIPSAPIEPENSPSVVAINVSPIEYGHVLLIPRILEHLPQRIDRESFLLALHMAAEAGNPYFRLGYNSLGAFATINHLHFQAYYLAVTFPIEKAPTKKITVSGAGVKVSELLNYPVRGLVFEGGNTLQDLSNTVSDACICLQENNIPYNVLISDCGKRIFLLPQCYAEKQALGEVRAELLDTQVNPAVWEISGHMVLKRKKDYEEASDENAWKLLAEVSLSEERFQEVNALIFEAIASDDNGNANLLEDPEVKPHSREEVDTINTSSHAAMVTGTQQCLVLQLSEEMKKGLHPQMQWISYVTQSGRLMHVMMTKIHHVGKVYHLKAKRQMTENLGQVAKFRQRYEKTAAPKEEP

SEQ ID NO: 223 - protein, 368 aa, Prunus dulcis
MVTVEQLQDDNFLSQCPPPLSLQGIRIPLYRFGSNSVLDNGSVGGISCLIEEEQSVLDSILLAQWEDRMWKGLFRYDVTTSEIKVIGGRRKFIAQFNEGWSRDCMPKLGKNKIRHQKDVFEFSWMERQEELLFCVASGEKLNPELVLSAAVPDCGLLITINSSPVEYGHVFLVPHSSYILHQFLDARSLELVLHVAVEVNNCSFRLFYDYSPSASHIYFQACYLQIPLPVEFMPLDTFFGDGQEGMRISSVTDYPIKTLVFESNHLNMMVGILAEICSCLQEKLIPYNLLISDHGKRIFLFFQLQTSKNFCTLSAWECGGYFVVKARSEFDQATEQALLKQLTTVSLDDEGFQGVKRLCCTIASKFAS

SEQ ID NO: 224 - protein, 437 aa, Solanum lycopersicum; identical to SEQ ID NO: 159

SEQ ID NO: 225 - protein, 438 aa, Solanum lycopersicum; identical to SEQ ID NO: 161

SEQ ID NO: 226 - protein, 357 aa, Oryza sativa
MGNRSFDLSCSLRKEEENGTNLSPFVRKLFKEWDDRKARGLFHHDISSCETKVLPGEHNFVATLIEGRDQKKRPTQFGMNQVLQPFDSVKFNFTKVSPEEVIFTFKESQNDSVKYFDNVPHAVAASPTAILINVSPIGYCHVLLIPRIQDCLPQRVDKESFLLAMYVASEAKDPFFRVGYNSLGGFATINHLHFQAYYLKVQYPVEKALTEKLTTLGNGVSIIQLVQYPVSGFVFEGGACLEDLSDVVSKVCIFLQENNKPFNALISESGKRVFLLPQCYAEKQALGRVSQEFLDMRINPAVWELSGHLVLKRRKDYDEASEATICRFLVEASLSESEFQELKSCILEFLSSAAPEE

SEQ ID NO: 227 - protein, 393 aa, Oryza sativa
MPISPLLAHLAPSSVAPAAFGARPARRRFVAERKTGMGNRSFDLSCSLRKEEENGTNLSPFVRKLFKEWDDRKARGLFHHDISSCETKVLPGEHNFVATLIEGRDQKKRPTQFGMNQVLQPFDSVKFNFTKVSPEEVIFTFKESQNDSVKYFDNVPHAVAASPTAILINVSPIGYCHVLLIPRIQDCLPQRVDKESFLLAMYVASEAKDPFFRVGYNSLGGFATINHLHFQAYYLKVQYPVEKALTEKLTTLGNGVSIIQLVQYPVSGFVFEGGACLEDLSDVVSKVCIFLQENNKPFNALISESGKRVFLLPQCYAEKQALGRVSQEFLDMRINPAVWELSGHLVLKRRKDYDEASEATICRFLVEASLSESEFQELKSCILEFLSSAAPEE

SEQ ID NO: 228 - protein, 368 aa, Oryza sativa
MVSTTEFKGEYSLPSKKSPLDQFEGVKTHLYRLGAEHENGTLKSFAYTDQGSPSLLDTIILSQWEDYAWKGHFGYDVTACNLKVVEGGWSFVVQLNDKWNSCVLKEHDKFLEPVGCLKPNCMNSYDELLLCIAQGDKDIPEVVPSTKPPKDGLLLIANAYPVEYGHIFLVPSATNQLSFFWDKRMFSLIARIASEVNSAAFRVFFDSCTSTMPDHMFFQACYFANPLPVESASTVAIYHGKATSAVHLYEIIDYPMKALVLTGKDVNTLANFVSEVSLTLHDNNTAYSLLISNNGTKVFLFPQVKNLATGCCLSAWECSGYFIYRAKYDFDRASENEISNRMASVTLQDGAFENLKNLCCAVADDLVM

SEQ ID NO: 229 - protein, 438 aa, Oryza sativa
MEMKLTIKRVPTVVSNYQEDAAATAGERPRAGCGRDCLGDCCLPDSKLPLYAFKASPKKPSSQEDASNDEFFVNLLLGLWEDRMARGLFRYDVTACETKVIPGNLGFVAQLNEGRHLKKRPTEFRVDRVLQPFDAAKFNFTKVGQEEVLFQFENGGGDDSFFVESSPISVADRAPNVVAINVSPIEYGHVLLIPRVLDRLPQRIDQESFLLALHMAAEAASPYFRLGYNSLGAFATINHLHFQAYYLTVPFPVEKAATKRIFLAEGTMNSGVKVSKLMNYPVRGLVFEGGNSLSDLANVVSSACIWLQDNNVPYNVLISDCGKKIFLFPQCYAEKQALGEVSQELLDTQVNPAVWEISGHIVLKRRSDYEEASEASAWRLLAEVSLSEERFEEVKAYIFDAAGLVQSDEEEVSEDEDATYTPVSIAPPAVAEGCLVLQ

SEQ ID NO: 230 - protein, 357 aa, Oryza sativa; identical to SEQ ID NO: 226

SEQ ID NO: 231 - protein, 357 aa, Oryza sativa; identical to SEQ ID NO: 226

SEQ ID NO: 232 - protein, 450 aa, Actinidia chinensis
MLKIKRVPTVVSNFQKDEAEDGARSGGGCGRNCLQKCCIQGAKLPLYAFKRVKEVVGEKGLLAVDDEEAPVAFLDSLLLGEWEDRVQRGLFRYDVTACETKVIPGEYGFIAQLNEGRHLKKRPTEFRVDKVLQPFDESKFNFTKVGQEEVLFQFEASDDNEVQFFPNAPVDVENSPSVVAINVSPIEYGHVLLIPRILECLPQRIDRESFLLALHMAAEAGNPYFRLGYNSLGAFATINHLHFQAYYLAVPFPIEKAPTRKITTLNGGVKISDLLNYPVRGLVFEGGNSLEDLSNAVSDSSICLQGNNIPYNVLISDSGKCIFLLPQCYAEKQALGEVSSDLLDTQVNPAVWEISGHMVLKRKEDYEEASEGNAWRLLAEVSLSEERFEEVKALIFEAISCADDRSGSTAENLLEEPDDNPQSRKVANDALNKGSHRGMVPGKQECLVQH

SEQ ID NO: 233 - protein, 445 aa, Malus domestica
MLRIKRVPTVVSNYQKDEAEEVARRVGGCGRNCLNQCCIPGAKLPLYAFKKLNVNDGDTGLLGREKREPPVAFLDSLLLGEWEDRMQRGLFRYDVTACETKVIPGQFGFIAQLNEGRHLKKRPTEFRVDKVLQPFDGSKFNFTKVGQEEVLFQFEASKDGEVQFFPSAPIDVESSPSVVAINVSPIEYGHVLLIPHILERLPQRIDRESFLLALHMAAEAGNPYFRLGYNSLGAFATINHLHFQAYYLAVTFPIEKAPTKKISTLNAEVKVSELLNYPVRGLVFEGGNTLQDLSNTVSDACICLQENNIPYNVLISDSGKRIFLVPQCYAEKQALGEVRAEILDTQVNPAVWEISGHMVLKRKKDYDEASDENAWKLLAEVSLSEERFLEVNALIFEGIASGDNGNENLLKDPEVKPRSHEEVNTINKRVHCSAVNEQICRVFTM

SEQ ID NO: 234 - protein, 450 aa, Actinidia chinensis
MLKIKRVPTVVSNFQKDEADDGARSGGGCGRNCLQKCCIQGAKLPLYAFKRVNEVVGEKGVLALDNEEAPVAFLDSLLLGEWEDRVQRGLFRYDVTACETKVIPGEYGFIAQLNEGRHLKKRPTEFRVDKVLQPFDGSKFNFTKVGQEEVLFQFEASNDNEVQFFPNAPVDVENSPSVVAINVSPIEYGHVLLIPSILECLPQRIDRESFLLALHMAAEAGNPYFRLGYNSLGAFATINHLHFQAYYLAVPFPIEKAPTRKITTLNGGVKISELLNYPVRGLVFEGGNTLEDLSNAVSDSSICLQGNNIPYNVLISDSGKRIFLLPQCYAEKQALGEVSSELLDTQVNPAVWEISGHMVLKRKEDYEEASEGNAWRLLAEVSLSGERFEEVKALIFEAISCADDRSSSTAENLLEEPDDNPQSREEANDALNKGSHCGMVPGKQECLVQH

SEQ ID NO: 235 - protein, 415 aa, Actinidia eriantha
MLKIKRVPTVVSNFQKDEAEDGARSGGGCGRNCLQKCCIQGAKLPLYAFKRVKEVVGEKGLLTVGDEEAPVAFLDSLLLGEWEDRVQRGLFRYDVTACQTKVIPGEYGFIAQLNEGRHLKKRPTEFRVDKVLQPFDESKFNFTKVGQEEVLFQFEASVDNEVQFFPNAPVDVENSPSVVAINVSPIEYGHVLLIPRILECLPQRIDRESFLLALHMAAEAGNPYFRLGYNSLGAFATINHLHFQAYYLAVPFPIEKAPTRKITTLNGGVKISELLNYPVRGLVFEGGNSLEDLSNAVSDSSICLQCNNIPYNVLISDSGKRIFLLPQCYAEKQALGEVSSELLDTQVNPAVWEISGHMVLKRKEDYQEASEGNAWRLLAEVSLSEERFEEVKALIFEAISCADDRSGSTAENLLE

SEQ ID NO: 236 - protein, 450 aa, Actinidia eriantha
MLKIKRVPTVVSNFQKDEAEDGARSGGGCGRNCLQKCCIQGAKLPLYAFKRVKEVVGEKGLLAVGDEEAPVAFLDSLLLGEWEDRVQRGLFRYDVTACETKVIPGEYGFIAQLNEGRHLKKRPTEFRVDKVLQPFDESKFNFTKVGQEEVLFQFEASIDNEVQFFPNAPVDVENSPSVVAINVSPIEYGHVLLIPRILECLPQRIDRESFLLALHMAAEAGNPYFRLGYNSLGAFATINHLHFQAYYLAVPFPIEKAPTRKITTLNGGVKISELLNYPVRGLVFEGGNSLEDLSNAVSDSSICLQCNNIPYNVLISDSGKRIFLLPQCYAEKQALGEVSSELLDTQVNPAVWEISGHMVLKRKEDYQEASEGNAWRLLAEVSLSEERFEEVKALIFEAISCADDRSGSTAENLLEEPDNDPQSREVANDALSKASHRGMVPGKQECLVQH

SEQ ID NO: 237 - protein, 447 aa, Malus sieboldii
MMLRIKRVPTVFSNYQKDEAEEGARRVEGCGRNCLNQCCIPGAKLPLYAFKKRNVNNGDTGVPGHDKREPPVAFLDSLLLGEWEDRMQRGLFRYDVTACETKVIPGQYGFIAQLNEGRHLKKRPTEFRVDKVLQPFDSSKFNFTKVGQEEVLFRFEASEDGEVHFFPSAPIDVENSPSVVAINVSPIEYGHVLLIPRIFERLPQRIDRESFLLALHMAAEAGSPYFRLGYNSLGAFATINHLHFQAYYLAVTFPIEKAPTKKISTLNAEVKVSELLNYPVRGLFFEGGNTLEDLSYTVSDACICLQENNVPYNVLISDCGKRIFLLPQCYAEKQALGEVSAEVLDTQVNPAVWEISGHMVLKRKKDYDEASDENAWKLLAEVSLSEERFQEVNALIFERIASGNNGNENLPEDPEVKPRSHEEVDATINKSSRAAMVGETQECIVLQ

SEQ ID NO: 238 - protein, 438 aa, Solanum tuberosum
MMLKIKRVPTLVSNFQKDEADEIGARGAGCGRNCLRNCCLPGSKLPLYGFKNLSYGKSVAADETKESPIDFLESLVLGEWEDRQQKGLFRYDVTACETKVIPGEYGFVAQLNEGRHLKKRPTEFRVDKVLQPFDGSKFNFTKVGQEELLFQFEASEEDEVQLYPNAPIDPEKSPSVIAINVSPIEYGHVLLIPKVLECLPQRIDRDSFLLALHMAAEAANPYFRLGYNSLGAFATINHLHFQAYFLAVQFPIEKAPTQKITVTDTGVKISEMLNYPVRGLVFEGGNTLEDLANVVSDSCICLQENNIPYNVLISDSGKRIFILPQCYAEKQALGEVSAELLDTQVNPAVWEISGHMVLKRKEDYEGATEANAWRLLAEVSLSEARFQEVTALIFEAISLSVEENENANDGSPEDLDVTPPQPMEEIDGSHTHSTMVPA

SEQ ID NO: 239 - protein, 442 aa, Arabidopsis thaliana; identical to SEQ ID NO: 157

SEQ ID NO: 240 - protein, 431 aa, Arabidopsis thaliana; identical to SEQ ID NO: 158

SEQ ID NO: 241 - protein, 438 aa, Solanum lycopersicum; identical to SEQ ID NO: 161

SEQ ID NO: 242 - protein, 438 aa, Oryza sativa; identical to SEQ ID NO: 229

(19) United States
(12) Patent Application Publication
(10) Pub. No.: US 2026/0049329 A1
(43) Pub. Date: Feb. 19, 2026

(54) UORF::REPORTER GENE FUSIONS TO SELECT SEQUENCE CHANGES TO GENE EDIT INTO UORFS TO REGULATE ASCORBATE GENES

(71) Applicants: Roger Paul Hellens, Ngongotaha (NZ); Oliver J. Ratcliffe, Alameda, CA (US); Jeffrey M. Libby, Cupertino, CA (US)

(72) Inventors: Roger Paul Hellens, Ngongotaha (NZ); Oliver J. Ratcliffe, Alameda, CA (US); Jeffrey M. Libby, Cupertino, CA (US)

(21) Appl. No.: 19/117,753

(22) PCT Filed: Oct. 4, 2023
(86) PCT No.: PCT/US2023/076024

§ 371 Date: Apr. 2, 2025

Related U.S. Application Data
(60) Provisional application No. 63/413,346, filed on Oct. 5, 2022.

(51) Int. Cl. C12N 15/82; C12N 9/12

(52) U.S. Cl. CPC C12N 15/8279; C12N 9/1241; C12N 15/8262; C12N 15/8273; C12Y 207/07069

(57) ABSTRACT

The present disclosure provides isolated polynucleotides comprising sequences encoding uORF peptides and variants and fragments thereof. The present disclosure also provides constructs and vectors containing the polynucleotides. The present disclosure further provides cells, plant cells and plants transformed with the polynucleotides and constructs. The present disclosure also provides methods of using the polynucleotides to control expression of operably linked polynucleotides. The present disclosure also provides methods of producing a plant cell or plant selected for a desirable trait, including at least one of increased ascorbate level, increased biomass, increased shoot number, decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and/or increased oxidative stress tolerance as compared to a control plant.

### FIELD OF THE INVENTION

The present disclosure is directed to the creation of mutations in upstream open reading frames (uORFs) that control DNA and protein expression and their uses in plants.

### BACKGROUND

Prior work has intensively focused on developing plants which have elevated levels of ascorbate (vitamin C) so as to provide a greater level of nutritional value upon consumption by humans and animals. However, the potential to deliver improved abiotic and/or biotic stress tolerance and/or an altered growth habit to crops by increasing the level of ascorbate in the crop cells has been less extensively explored. In particular, most previous work has been to genetically modify ascorbate levels in food crops; there remains substantial opportunity to create commercial non-food crops (e.g., crops grown for energy generation, carbon capture, chemical-processing, materials or fiber uses) that are protected from stress by expressing elevated ascorbate levels.

Ascorbate has been recognized as having an important role in protecting cells from the damaging effects of free radicals, which are produced when cells are under a state of oxidative stress. Plants are subject to many different types of stress throughout the life cycle, which result from environmental factors such as temperature, water and nutrient availability, light intensity, and the presence of pathogens. All of these factors have a common effect of increasing levels of oxidative stress and free radicals in plant cells, which in turn cause damage to macromolecules such as nucleic acids. In crops, this damage often leads to stunting, growth defects, and a low-quality harvested product. The invention detailed herein presents new crop traits and methods of creating such traits which protect against the deleterious effects of oxidative stress induced cellular damage, by elevating levels of ascorbate in plant cells by means of increasing the level of the GDP-L-GALACTOSE PHOSPHORYLASE (GGP) GENE product. This is achieved either through the generation of transgenic plants in which GGP is expressed from a heterologous promoter, or by introduction of mutations into the uORF, which has previously been shown to negatively regulate production of the GGP gene product. For the latter approach, the resulting alleles typically comprise mutations that do not either lie within the start codon or that substantially delete the uORF, and which produce only a moderate increase in ascorbate levels compared to a wild type plant. A number of authors have reported the generation of transgenic or gene edited plants in which elevated ascorbate levels have been achieved by modification of the GGP gene (For example, see: Koukounaras et al., Plant Physiology and Biochemistry 193, 15 Dec. 2022, 124-138; Yang et al., 2023 Protoplasma 260(2):625-635). When very high levels of ascorbate are produced, a drag on yield or negative effects such as stunting, altered shoot architecture, or organ abnormalities result. Thus, alleles which result in low to moderate elevation of ascorbate are those which are typically most desirable. In this specification, we represent novel reporter system-based approaches to identify desirable mutations in the uORFs of a GGP genes that deliver a desirable increase in GGP protein and/or ascorbate levels, without attendant deleterious effects on growth. Such changes or equivalent changes can then be recapitulated at an endogenous GGP gene in a target crop through gene editing or TILLING based approaches. However, in instances when the plant is an ornamental or a turf grass species, stunting and slow growth combined with stress tolerance may be highly desirable, in which case a strong allele would be selected

It should be noted that because GGP acts in a dosage dependent manner, alleles in which the uORF of GGP are disrupted, behave in a semi-dominant manner. Thus, a means of optimizing dosage, and removing undesirable negative phenotypes such as sterility or stunting is to make a genetic cross of a homozygous line carrying a uORF mutated allele of GGP with a heterozygous or wild-type line to produce an F1 heterozygote. Indeed, F1 heterozygous seed carrying such alleles may be preferable for commercial seed producers and/or growers.

The inventions disclosed herein can be applied with any GGP encoding gene since GPP has evolved as a key regulatory enzyme in plant ascorbate biosynthesis. Indeed, the enzyme is conserved throughout the Plant Kingdom: GGP genes were recently identified from more than 70 different species of plant by Tao et al., AoB Plants. 2020 Nov. 4;12(6): plaa055. Examples of GGP proteins are disclosed herein as SEQ ID NO: 157-242. However, the set of sequences presented is purely exemplary and is not intended to be limiting: the universe of GGPs to which these inventions can be applied is very broad and includes any functional homolog of disclosed GGP proteins.

An upstream open reading frame (uORF) is a member of a class of small, conserved ORFs located upstream of protein-coding major ORFs (mORFs) in the leader sequence [which is also known as the 5′-untranslated regions (5′UTR)] of mRNAs, uORFs act as cis acting elements that modify the activity of a downstream sequence that encodes the polypeptides. As such, they offer a novel opportunity to activate the expression of the downstream open reading frames encoding polypeptides of interest, through genetic manipulation, such as with gene editing approaches, that introduce mutations into the uORF sequences.

uORF regulatory elements are prevalent in eukaryotic mRNAs. However, not all eukaryotic genes contain uORFs. In some instances, uORFs are believed to modulate the translation initiation rate of downstream coding sequences (CDSs) by sequestering ribosomes. In other cases, uORFs encode evolutionarily conserved short peptides that function as cis-acting repressor peptides of the downstream mORF. In many cases the actual presence of a uORF is strongly conserved across species. Thus, once a uORF has been identified in a target locus from a given species, the homologous locus from another species will typically also contain a uORF and be subject to uORF repression.

Genome-wide studies have revealed the widespread regulatory functions of uORFs in different species in different biological contexts (Zhang et al. 2019. Trends Biochem. Sci. 44:782-794. doi: 10.1016/j.tibs.2019.03.002). A given uORF may act as a translational control element for regulating expression of its associated downstream major open reading frame (mORF). The translational regulation of mORFs by highly conserved uORFs in response to cellular metabolite levels has been documented in plant studies (Hayden C.A, and Jorgensen R.A. 2007. BMC Biol. 5:32; Tran M.K., et al. 2008. BMC Genomics 9:361). Upregulation of the level of a target polypeptide can thereby be achieved by modulating the expression, for example, by knocking-out, of a negatively acting uORF upstream of the sequence encoding the polypeptide. Genetic modifications are targeted to the uORF sequences to produce new desirable phenotypes that have a variety of applications depending upon the particular cell or organism. Such applications include new crop traits (e.g., increased vigor, stress tolerance, delayed or accelerated flowering, altered morphology, increased shoot number, reduced apical dominance, increased yield, and/or improved nutritional content), control of weeds and other pests through activation of gene networks that switch on cell death, activation of cell-death or tumor suppressor genes in cancerous cells, and/or production of desirable metabolites or peptides in fermentation systems.

The present disclosure relates to methods and compositions for producing commercially valuable plants and crops as well as the methods for making them and using them.

The uORFs provided and characterized in this disclosure may be modified for the purpose of producing plants with modified traits, particularly traits that address agricultural, food-production and material-production needs as well as needs for environmental rehabilitation and carbon sequestration. For example. GGP gene activity may be regulated by gene editing of polynucleotides encoding uORF peptides identified by Liang et al. (U.S. Pat. No. 9,648,813).

These traits may provide significant value in that they allow the plant to thrive in hostile environments, where, for example, temperature, water and nutrient availability or salinity may limit or prevent growth of plants lacking the modified traits. The traits may also comprise desirable morphological alterations, including alterations of flowering time, larger or smaller size, disease and pest resistance, light response, alterations in biochemical composition, and other desirable phenotypes. In particular, with growing interest in producing crops under controlled indoor conditions, traits such as delayed flowering or more compact architecture are often desirable, particularly in leafy greens.

Other aspects and embodiments of the disclosure are described below and can be derived from the teachings of this disclosure as a whole.

### SUMMARY

The present disclosure is directed to methods for producing the plants that upregulate, augment, or increasing a desirable trait in a plant. The plant or a cell of the plant may be ornamental, turf, weed, or crop. The present disclosure is also directed to plants produced by the disclosed methods. The plant with an upregulated desirable trait is modified by introducing a nucleic acid comprising a 5′ UTR (untranslated region) obtained from a region upstream of a gene of interest that directly or indirectly upregulates the desirable trait. The 5′ UTR comprises a mutation in the upstream open reading frame (uORF). The 5′UTR may also be characterized in that it comprises at least one of:
i) a sequence with at least 70% identity to any one of SEQ ID NOs: 41-100, 111-131, 144-145, 147, 149-150, or 153-155; and
ii) ii) a sequence encoding a polypeptide with at least 70% identity to any one of SEQ ID NOs: 1-40, 108, 132-137, 146, 148, 151-152, or 156; wherein the mutation disrupts the function of an upstream open reading frame (uORF) encoded by the 5′UTR; and wherein said modification is at least one of a deletion, an addition, or a substitution of at least one nucleotide in the 5′UTR.

The mutation may be introduced by a number of means known in the art including but not limited to irradiation, photomutagenesis, or chemical mutagenesis. The mutation may also be created by a targeted gene edit wherein the sequence change produced through the edit was previously chosen or selected through a process in which the mutated 5′UTR is fused to a polynucleotide encoding a reporter protein, for example, luciferase (LUC). B-glucuronidase (GUS, or green fluorescent protein (GFP). The resulting fusion is introduced into a test plant or plant cell or protoplast

Typically, a fusion where the uORF function has been completely removed, produces a substantially higher level (in some cases many fold) of the reporter product than a reporter fusion in which the entire 5′ UTR remains intact. This system can thus be used to dissect the functions of the different regions within the uORF contained within a 5′UTR. In many cases the practitioner desires to select a “weak” mutation within the uORF that releases repression of the uORF, but does not do so completely, and thereby delivers a reduced level of reporter product compared to when the uORF function is fully removed. In such instances the level of a product of the reporter gene in the test plant or plant cell or protoplast is about 10%, about 20%, about 30%, about 40%, about 50%, about 60%, about 70%, about 80%, or about 90% of the level of a level of reporter product produced in a control plant or plant cell or protoplast comprising a control 5′ UTR:reporter polynucleotide in which the uORF has been rendered inoperable by its partial or complete deletion from the 5′ UTR fused to the reporter gene.

Once a desired mutation within a uORF has been selected via the reporter protein analysis, the practitioner then uses gene editing to introduce an equivalent or comparable mutation into the 5′UTR of a gene of interest at its endogenous locus in the genome of a plant species in which the practitioner desires to produce a desirable trait. This typically involves introducing a guide nucleic acid, which targets the endogenous locus where the selected edit is desired, into cells of a plant of said species and then regenerating plants via tissue culture that contain novel alleles that result from the editing process. An individual plant may be selected for the desirable trait from a pool of plants that comprise the nucleic acid changes in the uORF. The desirable traits referred to in this specification (one of at least one of a present “desirable trait”) may include but are not limited to at least one of increased decreased free radical damage, decreased free radical level, increased stress tolerance, increased abiotic stress tolerance (e.g. one or more of salt tolerance, radiation tolerance, pollution tolerance, heat tolerance, cold tolerance and/or drought tolerance) increased biotic stress tolerance, increased disease tolerance, increased disease resistance, increased nutrient use efficiency, increased nitrogen use efficiency, increased shoot number. and/or increased oxidative stress tolerance.

The present disclosure also provides a method for producing a plant cell or plant selected for a present desirable trait compared to a control plant. The method includes modification of the 5′-UTR of a GGP gene in the plant cell or plant, wherein the 5′UTR comprises at least one of a sequence with at least 70% identity to any one of SEQ ID NOs: 41-100, 111-131, 144-145, 147, 149-150, or 153-155; or a sequence encoding a polypeptide with at least 70% identity to any one of SEQ ID NOs: 1-40, 108, 132-137, 146, 148, 151-152, or 156; wherein the modification disrupts the function of an upstream open reading frame (uORF) encoded by the S′UTR; and wherein said modification is at least one of a deletion, an addition, or a substitution of at least one nucleotide in the 5′UTR.

The instant specification is also directed to a method for upregulating a desirable trait in a plant. The method provides a nucleic acid comprising a 5′ UTR (untranslated region) obtained from a region upstream of a gene of interest that directly or indirectly upregulates the desirable trait, wherein the 5′ UTR comprises an upstream open reading frame (uORF). The 5′UTR comprises at least one of:
i) a sequence with at least 70% identity to any one of SEQ ID NOs: 41-100, 111-131, 144-145, 147, 149-150, or 153-155; and
ii) ii) a sequence encoding a polypeptide with at least 70% identity to any one of SEQ ID NOs: 1-40, 108, 132-137, 146, 148, 151-152, or 156; wherein the modification disrupts the function of an upstream open reading frame (uORF) encoded by the 5′UTR; and wherein said modification is at least one of a deletion, an addition, or a substitution of at least one nucleotide in the 5′UTR.

The nucleic acid is fused to a reporter gene (for example, LUC, GUS, or GFP) to create a 5′ UTR::reporter polynucleotide. A mutation is introduced into the nucleic acid (for example, by direct synthesis) to create a 5′ UTR:reporter polynucleotide with the mutation in the uORF or putative uORF. The 5′ UTR:reporter polynucleotide is introduced into a plant cell. A level of a product of the reporter gene is then measured in the plant cell. Subsequently, a mutation is selected or identified that produces less of the product in the plant cell than the level of the product in a control plant cell comprising a control 5′ UTR:reporter polynucleotide in which the uORF has been deleted from the 5′ UTR fused to the reporter gene. The level of the product in the plant cell may be about 10%, about 20%, about 30%, about 40%, about 50%, about 60%, about 70%, about 80%, or about 90%, or less than 100% of the level of the reporter product produced in the control plant. The selected mutation in the uORF which produced the desired change in the level of the reporter product is then introduced into the genome of a cell of a crop plant that is subsequently regenerated into a plant into a crop plant exhibiting an upregulation of the desirable trait. The resulting plant carrying the upregulated desired trait is then used for breeding, to introduce the trait into commercial germplasm, or it is subjected to vegetative propagation, to produce a population of plants carrying the same allele and trait. Typically, for breeding a plant carrying a desired allele that delivers a desired trait will be backcrossed through up to 8 generations to a desired variety in order to “fix” the allele in the genetic background of the desired variety. As used herein, “desirable trait refers to but is not limited to at least one of increased decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, increased shoot number, altered aerial architecture, and/or increased oxidative stress tolerance.

### BRIEF DESCRIPTION OF THE SEQUENCE LISTING

The Sequence Listing provides exemplary polynucleotide and polypeptide sequences of the instant disclosure. The Sequence Listing is named GXTR-0001PCT.xml, was created on 3 Oct. 2023, and is 319,872 bytes in size. Where required, the entire content of the Sequence Listing is hereby incorporated by reference.

### DETAILED DESCRIPTION

### Definitions

“Expression” as used herein, refers to the production of mRNA from a gene via transcription or the production of a polypeptide from an RNA via translation.

“uORFs” are upstream open reading frames, that often reside in the leader sequence of an mRNA transcript located upstream of protein-coding main ORFs (Note that mORFs are also sometimes referred to as long ORFs or major ORFs and the terms mORF, main ORF, long ORF and major ORF are used interchangeably in this application), uORFs are a class of small ORFs that acts as repressors of their downstream mORFs, uORFs sometimes encode evolutionarily conserved functional peptides such as cis-acting regulatory peptides and which act as repressors, including for example, through translational repression, uORFs are generally defined by a start codon (any three base pair codon with at least two of the following bases in order: AUG) in the 5′-UTR, with an in frame stop codon (UAA, UAG, UGA), that is upstream (i.e., in a 5′ direction) and not overlapping with the main coding sequence.

“5′UTR” means “5′ Untranslated Region” and in the context used herein, the term refers to the leader sequence at the 5′ end of an mRNA molecule, upstream of the main ORF. It should be noted “untranslated” is in some instances a misnomer as there are instances where a uORF residing in the 5′UTR is itself translated into a short peptide.

A “polypeptide” is a sequence of amino acids comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Identity” or “similarity” refers to sequence similarity between two polynucleotide sequences or between two polypeptide sequences, with identity being a stricter comparison. The phrases “percent identity” and “% identity” refer to the percentage of sequence identity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

The term “homolog” or “homologue” as further described and used herein means a variant polypeptide or transcription factor from the same species or a different species which has a substantial level of identity within either its conserved domain and/or across its entire sequence, wherein the level of identity is at least 30% or at least 35%, or at least 40%, or at least 45%, or at least 50%, or at least 55%, or more preferably at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity as compared to a first polypeptide or transcription factor and which polypeptide or transcription factor has a similar or comparable function in a cell or organism as compared to the first polypeptide or transcription factor. Furthermore, homologous nucleotide sequences will hybridize under stringent conditions.

“GGP protein” as described herein means the family of proteins produced by genes that encode GDP-L-galactose phosphory lases in plants, as exemplified by any protein product encoded by the Arabidopsis locus AT4G26850 and the Arabidopsis VTC5 locus AT5G55120 (each an “Arabidopsis GGP protein) including two protein sequences represented by SEQ ID NO: 239 and SEQ ID NO: 240.

“Homolog” as described herein in the context of a GGP protein means a polypeptide, which upon performing a BLAST analysis against the set of proteins encoded by the Arabidopsis proteome, returns a higher level of sequence identity to an Arabidopsis GGP protein than to any other protein in the Arabidopsis proteome, and which has a level of similarity to an Arabidopsis GGP protein equal or higher than an HSP of bit score 50.

“Orthologs” are evolutionarily related genes that have similar sequence and similar functions. Orthologs are structurally related genes in different species that are derived by a speciation event.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogs and orthologs. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein (Liang et al. U.S. Pat. No. 9,648,813).

An “inverted repeat” is a sequence that is repeated, where the second half of the repeat is in the complementary strand of DNA. In such cases, read-through transcription will produce a transcript that undergoes complementary base-pairing to form a “hairpin” structure provided that there is a 3-5 bp spacer between the repeated regions.

A “non-food” plant refers to, for example but not limited to, a turf plant, an ornamental plant, or a crop that is grown for carbon capture or to produce manufactured goods, for example, fiber, biofuels, specialty chemicals, lubricants, building materials, pharmaceuticals, or biopolymers.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present disclosure. Identity is found over a comparison window of at least 20 nucleotide positions, preferably at least 50 nucleotide positions, more preferably at least 100 nucleotide positions, and most preferably over the entire length of a polynucleotide of the disclosure.

Polynucleotide sequence identity can be determined in the following manner. The subject polynucleotide sequence is compared to a candidate polynucleotide sequence using BLASTN (from the BLAST suite of programs, version 2.2.5 [November 2002]) In bl2seq (Tatiana A. Tatusova, Thomas L. Madden (1999), “Blast 2 sequences—a new tool for comparing protein and nucleotide sequences”, FEMS Microbiol. Lett. 174:247-250), which is publicly available from NCBI (ftp colon slash slash file transfer protocol.ncbi.nih.gov/blast/). The default parameters of bl2seq are utilized except that filtering of low complexity parts should be turned off.

The identity of polynucleotide sequences may be examined using the following Unix command line parameters: bl2seq -i nucleotideseq1 -j nucleotideseq2 -F F -p blastn

The parameter-F F turns off filtering of low complexity sections. The parameter-p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g., Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from worldwide web.hgmp.mrc.ac.uk/Software/EMBOSS/. The European Bioinformatics Institute server also provides the facility to perform EMBOSS-needle global alignments between two sequences on line at worldwide web.ebi.ac.uk/emboss/align/.

Alternatively, the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

A preferred method for calculating polynucleotide % sequence identity is based on aligning sequences to be compared using Clustal X (Jeanmougin et al., 1998, Trends Biochem. Sci. 23, 403-5.)

Polynucleotide variants of the present disclosure also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp colon slash slash file transfer protocol ftp.ncbi.nih.gov/blast/).

The similarity of polynucleotide sequences may be examined using the following Unix command line parameters: bl2seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10−6 more preferably less than 1×10−9, more preferably less than 1×10−12, more preferably less than 1×10−15, more preferably less than 1×10−18, more preferably less than 1×10−21, more preferably less than 1×10−30, more preferably less than 1×10−40, more preferably less than 1×10−50, more preferably less than 1×10−60, more preferably less than 1×10−70, more preferably less than 1×10−80, more preferably less than 1×10−90 and most preferably less than 1×10−100 when compared with any one of the specifically identified sequences.

Alternatively, variant polynucleotides of the present disclosure hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C−log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6× Saline-Sodium Citrate (SSC), 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C., below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037);1497-500) Tm values are higher than those for DNA-DNA or DNA-RNA hybrids, and can be calculated using the formula described in Giesen et al., Nucleic Acids Res. 1998 Nov. 1: 26(21):5004-6. Exemplary stringent hybridization conditions for a DNA-PNA hybrid having a length less than 100 bases are 5 to 10° C. below the Tm.

Variant polynucleotides of the present disclosure also encompass polynucleotides that differ from the sequences of the disclosure but that, as a consequence of the degeneracy of the genetic code, encode a polypeptide having similar activity to a polypeptide encoded by a polynucleotide of the present disclosure. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the present disclosure. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [Nov. 2002]) from NCBI (ftp colon slash slash file transfer protocol ftp.ncbi.nih.gov/blast/) via the tblastx algorithm as previously described.

The function of a variant polynucleotide of the disclosure may be assessed, for example, by cloning such a sequence in bacteria and testing activity of the encoded protein as described in the Example section. Function of a variant may also be tested for its ability to alter GGP activity, ascorbate content, a desirable trait, or content of useful compounds, nutrients, or components in plants, also as described in the Examples section herein. (Liang et al. U.S. Pat. No. 9,648,813).

### Polypeptide Variants

The term “variant” with reference to polypeptides encompasses naturally occurring, recombinantly and synthetically produced polypeptides. Variant polypeptide sequences preferably exhibit at least 30%, at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present disclosure. Identity is found over a comparison window of at least 20 amino acid positions, preferably at least 50 amino acid positions, more preferably at least 100 amino acid positions, and most preferably over the entire length of a polypeptide of the disclosure.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from NCBI (ftp colon slash slash file transfer protocol ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at world wide web.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5.)

Polypeptide variants of the present disclosure also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp colon slash slash file transfer protocol ftp.ncbi.nih.gov/blast/). The similarity of polypeptide sequences may be examined using the following Unix command line parameters: bl2seq -i peptideseq1 -j peptideseq2 -F F -p blastp

Variant polypeptide sequences preferably exhibit an E value of less than 1×10−6 more preferably less than 1×10−9, more preferably less than 1×10−12, more preferably less than 1×10−15, more preferably less than 1×10−18, more preferably less than 1×10−21, more preferably less than 1×10−30, more preferably less than 1×10−40, more preferably less than 1×10−50, more preferably less than 1×10−60, more preferably less than 1×10−70, more preferably less than 1×10−80, more preferably less than 1×10−90 and most preferably 1×10−100 when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

A variant polypeptide includes a polypeptide wherein the amino acid sequence differs from a polypeptide herein by one or more conservative amino acid substitutions, deletions, additions, or insertions which do not affect the biological activity of the peptide. Conservative substitutions typically include the substitution of one amino acid for another with similar characteristics, e.g., substitutions within the following groups: valine, glycine, alanine; valine, Isoleucine, leucine; aspartic acid, glutamic acid; asparagines, glutamine; serine, threonine; lysine, arginine; and phenylalanine, tyrosine.

Non-conservative substitutions will entail exchanging a member of one of these classes for a member of another class.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences, one example being the BLOSUM62 matrix shown below (Table 1).

TABLE 1
The BLOSUM62 matrix containing all possible substitution
scores [Henikoff and Henikoff, 1992].

| | A | R | N | D | C | Q | E | G | H | I |
|---|---|---|---|---|---|---|---|---|---|---|
| A | 4 | −1 | −2 | −2 | 0 | −1 | −1 | 0 | −2 | −1 |
| R | −1 | 5 | 0 | −2 | −3 | 1 | 0 | −2 | 0 | −3 |
| N | −2 | 0 | 6 | 1 | −3 | 0 | 0 | 0 | 1 | −3 |
| D | −2 | −2 | 1 | 6 | −3 | 0 | 2 | −1 | −1 | −3 |
| C | 0 | −3 | −3 | −3 | 9 | −3 | −4 | −3 | −3 | −1 |
| Q | −1 | 1 | 0 | 0 | −3 | 5 | 2 | −2 | 0 | −3 |
| E | −1 | 0 | 0 | 2 | −4 | 2 | 5 | −2 | 0 | −3 |
| G | 0 | −2 | 0 | −1 | −3 | −2 | −2 | 6 | −2 | −4 |
| H | −2 | 0 | 1 | −1 | −3 | 0 | 0 | −2 | 8 | −3 |
| I | −1 | −3 | −3 | −3 | −1 | −3 | −3 | −4 | −3 | 4 |
| L | −1 | −2 | −3 | −4 | −1 | −2 | −3 | −4 | −3 | 2 |
| K | −1 | 2 | 0 | −1 | −3 | 1 | 1 | −2 | −1 | −3 |
| M | −1 | −1 | −2 | −3 | −1 | 0 | −2 | −3 | −2 | 1 |
| F | −2 | −3 | −3 | −3 | −2 | −3 | −3 | −3 | −1 | 0 |
| P | −1 | −2 | −2 | −1 | −3 | −1 | −1 | −2 | −2 | −3 |
| S | 1 | −1 | 1 | 0 | −1 | 0 | 0 | 0 | −1 | −2 |
| T | 0 | −1 | 0 | −1 | −1 | −1 | −1 | −2 | −2 | −1 |
| W | −3 | −3 | −4 | −4 | −2 | −2 | −3 | −2 | −2 | −3 |
| Y | −2 | −2 | −2 | −3 | −2 | −1 | −2 | −3 | 2 | −1 |
| V | 0 | −3 | −3 | −3 | −1 | −2 | −2 | −3 | −3 | 3 |

The BLOSUM62 matrix shown is used to generate a score for each aligned amino acid pair found at the intersection of the corresponding column and row. For example, the substitution score from a glutamic acid residue (E) to an aspartic acid residue (D) is 2. The diagonal show scores for amino acids which have not changed. Most substitutions changes have a negative score. The matrix contains only whole numbers.

Determination of an appropriate scoring matrix to produce the best alignment for a given set of sequences is believed to be within the skill of in the art. The BLOSUM62 matrix in Table 1 is also used as the default matrix in BLAST searches, although not limited thereto.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g., D-amino acids or non-naturally occurring synthetic amino acids, e.g., beta or gamma amino acids and cyclic analogs

The function of a polypeptide variant as a GGP may be assessed by the methods described in the Example section herein.

The function of a polypeptide variant as a GDP-D-Mannose epimerase may be assessed by the methods described in the Example section herein. (Liang et al. U.S. Pat. No. 9,648,813).

### Constructs, Vectors and Components Thereof

The term “genetic construct” or “construct” refers to a polynucleotide molecule, usually double-stranded DNA, which may have inserted into it another polynucleotide molecule (the insert polynucleotide molecule) such as, but not limited to, a cDNA molecule.

A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be in the form of a plasmid and/or be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction: a) a promoter functional in the host cell into which the construct will be transformed, b) the polynucleotide to be expressed, and c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequence of interest, such as a sequence to be expressed is placed under the control of, and typically connected to another sequence comprising regulatory elements that may include promoters, tissue-specific regulatory elements, temporal regulatory elements, enhancers. repressors and terminators, 5′-UTR sequences, 5′-UTR sequences comprising uORFs, and uORFs.

In a preferred embodiment the regulatory elements include a polynucleotide sequence of the disclosure.

Preferably the sequence of the present disclosure comprises a 5′-UTR sequence. Preferably the 5′-UTR sequence comprises a uORF.

The term “noncoding region” refers to untranslated sequences that are upstream of the translational start site and downstream of the translational stop site. These sequences are also referred to respectively as the 5′-UTR and the 3′-UTR. These regions include elements required for transcription initiation and termination and for regulation of translation efficiency.

A 5′-UTR sequence is the sequence between the transcription initiation site, and the translation start site of a main ORF.

The 5′-UTR sequence is an mRNA sequence encoded by the genomic DNA. However as used herein the term 5′-UTR sequence includes the genomic DNA sequence encoding the 5′-UTR sequence, and the complement of that genomic sequence, and the 5′-UTR mRNA sequence.

Terminators are sequences, which terminate transcription, and are found in the 3′ untranslated ends of genes downstream of the translated sequence. Terminators are important determinants of mRNA stability and in some cases have been found to have spatial regulatory functions.

The terms “to alter expression of” and “altered expression” of a polynucleotide or polypeptide of the present disclosure, are intended to encompass the situation where genomic DNA corresponding to a polynucleotide of the present disclosure is modified thus leading to altered expression or an altered level of a polynucleotide or polypeptide of the present disclosure. The level of the polynucleotide or polypeptide may be measured through one of a range of techniques including, transcript profiling, RT-PCR, Northern Blot, Western Blot, microarray analysis, RNASeq, or measurement of fluorescence or luminescence. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

The term “introduced targeted genetic modification” or “targeted genetic modification” refers to a change in the DNA sequence of a plant at a specific chromosomal position (also known as a locus) in the genome which is chosen by a skilled practitioner (such as plant breeder or molecular biologist) and which change is introduced by a process of gene editing and/or selection using a specific complementary nucleic acid molecule sequence as a guide or probe to enable the process.

The term “native genomic locus” or “endogenous locus” refers to a gene or DNA sequence that is present in the genome of a wild-type plant at particular chromosomal position of a given species. The “native genomic locus” typically comprises a region spanning a start to stop codon, along with any intervening introns, that is transcribed to generate a main ORF that encodes a long polypeptide that is typically around 100 amino acids or more in length, as well as the associated upstream regulatory elements including the promoter region and any elements that control the activity of the mORF such as uORFs. A uORF is present in the same mRNA transcript as the mORF that the uORF regulates; both the uORF and the mORF can therefore be considered part of the same overall native genomic locus. A native genomic locus is often specified by reference to an accession number, deposited in GenBank, which, for example, indicates the DNA sequence and encoded polypeptide that is present at that position. It should also be noted that a locus may encode multiple protein variants that result from alternative splicing of mRNA and these variants are represent by different “gene models” that are denoted by the accession number followed by a dot and a number. (Liang et al. U.S. Pat. No. 9,648,813).

The term “non-native allele of a gene” refers to a sequence variant of a gene (where the term gene includes both the protein coding region as well as upstream control elements such as uORFs) from a given plant species that results from a human intervention such as gene editing or selection and/or a sequence of nucleotides which is not found in nature in either the genome of a wild-type plant of that species or in the genome of a plant of that species taken from a naturally-occurring wild population.

The term “variant”, as used herein, may refer to polynucleotides or polypeptides, that differ from the presently disclosed polynucleotides or polypeptides, respectively, in sequence from each other, and as set forth above and below.

With regard to polynucleotide variants, differences between presently disclosed polynucleotides and polynucleotide variants are limited so that the nucleotide sequences of the former and the latter arc closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the present disclosure is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

The term “plant” includes whole plants, shoot vegetative organs/structures (e.g., leaves, stems, and tubers), roots, rhizomes, flowers, and floral organs/structures (e.g., bracts, sepals, petals, stamens, carpels, anthers, and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. The class of plants that can be used in the method of the present disclosure is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae. See for example, Daly et al. (2001) Plant Physiol. 127: 1328-1333; Ku et al. (2000) Proc. Natl. Acad. Sci. 97: 9121-9126; and see also Tudge, in The Variety of Life, Oxford University Press, New York, NY (2000) pp. 547-606.

A “trait” refers to a physiological, morphological, biochemical, or physical characteristic of a cell or organism, including of a plant or of a particular plant material or of a plant cell. In some instances, this characteristic is visible to the human eye, such as seed or plant size, or pigmentation, or can be measured by biochemical techniques, such as detecting the protein, starch, or oil content of seed or leaves, or by observation of a metabolic or physiological process, e.g., by measuring uptake of carbon dioxide, or by the observation of the expression level of a gene or genes, e.g., by employing Northern analysis. RT-PCR, microarray gene expression assays, RNA Seq or reporter gene expression systems, or by agricultural observations such as stress tolerance or pathogen tolerance. Any technique can be used to measure the amount of, comparative level of, or difference in any selected chemical compound or macromolecule in the transformed plants, however.

“Trait modification” refers to a detectable difference in a characteristic in a plant ectopically expressing a polynucleotide or polypeptide of the present disclosure relative to a plant not doing so, such as a wild-type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, at least about an 85%, or about a 100%, or an even greater difference compared with a wild-type plant. It is known that there can be a natural variation in the modified trait. Therefore, the trait modification observed entails a change of the normal distribution of the trait in the plants compared with the distribution observed in wild-type plant.

“Wild type” or “Wild-type”, as used herein, refers to a cell, tissue or plant that has not been genetically modified to knock out or overexpress one or more of the presently disclosed transcription factors. Wild-type cells, tissue or plants may be used as controls to compare levels of expression and the extent and nature of trait modification with cells, tissue or plants in which transcription factor expression is altered or ectopically expressed, e.g., in that it has been knocked out or overexpressed. A wild-type allele refers to the DNA sequence at a particular endogenous locus within the genome of a wild type plant.

A “crop” plant includes cultivated plants or agricultural produce, and may be a grain, vegetables, a fruit plant, an ornamental plant, or a plant used for energy production or production of a material, generally considered as a group. A crop plant may be grown in commercially useful numbers or amounts.

### DESCRIPTION OF THE SPECIFIC EMBODIMENTS

The present disclosure is directed to a method for improving, increasing, or upregulating a desirable trait in a plant. This method makes use of a nucleic acid that contains a 5′ UTR (untranslated region) with an upstream open reading frame (uORF) from a region upstream of a gene of interest. The nucleic acid is fused to a reporter gene to create a 5′ UTR::reporter polynucleotide which is then used to identify a useful mutation that can be introduced by gene editing or allelic selection into the uORF at its endogenous locus in the plant genome to modify the activity of a downstream protein coding ORF which the uORF regulates. Typically, the selected uORF mutation will increase the level of protein produced from the downstream ORF which in turn will result in the plant expressing a desirable trait such as tolerance to abiotic or biotic stress. The methodology begins by introducing a mutation into the nucleic acid in the uORF region or sequence of the 5′ UTR. The mutation may be introduced by a number of means known in the art including directly through DNA synthesis when the 5′ UTR::reporter polynucleotide is generated but could also be produced by other means, not limited to irradiation, photomutagenesis or chemical mutagenesis, DNA repair enzymes, or gene editing. The 5′ UTR::reporter polynucleotide is then introduced into a test cell or protoplast to examine its activity. The cell may be from a plant or alga but in some instances a microbial cell such as yeast may be used. The level of the reporter gene product is then measured in the cell, which level may be used to identify a mutation that produces more or less of the reported product in the plant cell than the level of the product in a control plant cell. The control plant cells may, for example, comprise a control 5′ UTR::reporter polynucleotide in which the uORF has been deactivated, for example, by complete or partial deletion in whole or in part from the 5′ UTR fused to the reporter gene. The mutation which increased the level of reporter product in the aforementioned reporter system is then introduced into the uORF (or the uORF of a homolog) in the genome of crop of interest or a cell of a target crop interest (which may be a food crop, a non-food crop, turf or an ornamental crop, or an experimental plant). Typically, the mutation is introduced either by the selection of an individual plant or cell carrying the mutation from a mutagenized population (by molecular genotyping; e.g., by PCR, followed by digestion or sequencing of the PCR product), or by gene editing. The cell or plant harboring the mutation is then as applicable, regenerated into a whole plant, and tested to verify that it harbors the trait of interest. Typically, a number of plants carrying the mutation (lines or events) will be created and then one or more will be selected for experimental or commercial use based on the strength of the desired trait that is exhibited.

The present disclosure also relates to plants and constructs containing the 5′ UTR:reporter polynucleotide with the mutation in the uORF.

In a particular embodiment of the above method, the gene of interest encodes a GGP protein, which, when its level increases, results a trait comprising an increase in the level of ascorbate in the plant, which provides an increased level of protection against abiotic or biotic stress. This, in turn, results in the plant exhibiting the trait of improved stress tolerance, which is typically associated with better growth and vigor, or a change in shoot architecture, or an increased number of shoots, reduced levels of anthocyanins, reduced damage to tissue, or reduced wilting as compared to a control plant.

In one aspect, the present disclosure provides an isolated polynucleotide comprising a sequence encoding a polypeptide with an amino acid sequence selected from SEQ ID NO: 1 to 20 and 132 to 134 (uORF peptides) or a variant or fragment thereof.

In one embodiment the variant or fragment comprises a sequence with at least 70% Identity to an amino sequence selected from SEQ ID NO:21 to 40 and 135 to 137 (conserved region of uORF peptides).

In a further embodiment the variant or fragment comprises a sequence with an amino acid selected from SEQ ID NO:21 to 40 and 135 to 137 (conserved region of uORF peptides).

In one embodiment the variant or fragment comprises a sequence with at least 70% Identity to an amino sequence selected from SEQ ID NO: 21-30, 33-37 and 135 to 137 (conserved region of dicot uORF peptides).

In a further embodiment the variant or fragment comprises a sequence with an amino acid selected from SEQ ID NO: 21 to 30, 33 to 37 and 135 to 137 (conserved region of dicot uORF peptides).

In one embodiment the variant or fragment comprises a sequence with at least 70% Identity to the amino sequence of SEQ ID NO: 108 (consensus motif uORF peptides).

In a further embodiment the variant or fragment comprises the amino sequence of SEQ ID NO: 108 (consensus motif uORF peptides).

In a further embodiment the variant comprises a sequence with at least 70% identity to an amino acid sequence selected from SEQ ID NO:1 to 20 and 132 to 134 (uORF peptides).

In a further embodiment the variant comprises an amino acid sequence selected from SEQ ID NO:1 to 20 and 132 to 134 (uORF peptides).

In a further embodiment the variant comprises a sequence with at least 70% identity to an amino acid sequence selected from SEQ ID NO: 1 to 10, 13 to 17 and 132 to 134 (dicot uORF peptides).

In a further embodiment the variant comprises an amino acid sequence selected from SEQ ID NO: 1 to 10, 13 to 17 and 132 to 134 (dicot uORF peptides).

In a further embodiment the isolated polynucleotide comprises a sequence with at least 70% Identity to a sequence selected from SEQ ID NO:61 to 80 and 138 to 140 (conserved region of uORF DNA sequences).

In a further embodiment the isolated polynucleotide comprises a sequence selected from SEQ ID NO: 61 to 80 and 138 to 140 (conserved region of uORF DNA sequences).

In a further embodiment the isolated polynucleotide comprises a sequence with at least 70% identity to a sequence selected from SEQ ID NO: 61 to 70, 73 to 77 and 138 to 140 (conserved region of dicot uORF DNA sequences).

In a further embodiment the isolated polynucleotide comprises a sequence selected from SEQ ID NO: 61 to 70, 73 to 77 and 138 to 140 (conserved region of dicot uORF DNA sequences).

In a further embodiment the isolated polynucleotide comprises a sequence with at least 70% Identity to a sequence selected from SEQ ID NO:41 to 60 and 129 to 131 (uORF DNA sequences).

In a further embodiment the isolated polynucleotide comprises a sequence selected from SEQ ID NO: 41 to 60 and 129 to 131 (uORF DNA sequences).

In a further embodiment the isolated polynucleotide comprises a sequence with at least 70% Identity to a sequence selected from SEQ ID NO: 41 to 50, 53 to 57 and 129 to 131 (dicot uORF DNA sequences).

In a further embodiment the isolated polynucleotide comprises a sequence selected from SEQ ID NO: 41 to 50, 53 to 57 and 129 to 131 (dicot uORF DNA sequences).

In a further embodiment the isolated polynucleotide comprises a sequence with at least 70% Identity to a sequence selected from SEQ ID NO:111 to 125 (5′-UTR sub-sequences).

In a further embodiment the isolated polynucleotide comprises a sequence selected from SEQ ID NO: 111 to 125 (5′-UTR sub-sequences).

In a further embodiment the isolated polynucleotide comprises a sequence with at least 70% Identity to a sequence selected from SEQ ID NO:81 to 100 and 126 to 128 (whole 5′-UTR sequences).

In a further embodiment the isolated polynucleotide comprises a sequence selected from SEQ ID NO: 81 to 100 and 126 to 128 (whole 5′-UTR sequences).

In a further embodiment the isolated polynucleotide comprises a sequence with at least 70% Identity to a sequence selected from SEQ ID NO: 81 to 90, 93 to 97 and 125 to 128 (whole dicot 5′-UTR sequences).

In a further embodiment the isolated polynucleotide comprises a sequence selected from SEQ ID NO: 81 to 90, 93 to 97 and 125 to 128 (whole dicot 5′-UTR sequences).

In a further aspect the present disclosure provides an isolated polynucleotide comprising a sequence selected from SEQ ID NO:41 to 60 and 129 to 131 (uORF DNA sequences) or a variant or fragment thereof.

In one embodiment the variant or fragment comprises a sequence with at least 70% identity to a sequence selected from SEQ ID NO:61 to 80 and 138 to 140 (conserved region of uORF DNA sequences).

In one embodiment the variant or fragment comprises a sequence with at least 70% identity to a sequence selected from SEQ ID NO: 61 to 70, 73 to 77 and 138 to 140 (conserved region of dicot uORF DNA sequences).

In a further embodiment the isolated polynucleotide comprises a sequence selected from SEQ ID NO: 41 to 60 and 129 to 131 (uORF DNA sequences).

In one embodiment the variant comprises a sequence with at least 70% identity to a sequence selected from SEQ ID NO:41 to 60 and 129 to 131 (uORF DNA sequences).

In one embodiment the variant comprises a sequence with at least 70% Identity to a sequence selected from SEQ ID NO: 41 to 50 and 53 to 57 and 129 to 131 (dicot uORF DNA sequences).

In a further embodiment the isolated polynucleotide comprises a sequence with at least 70% identity to a sequence selected from SEQ ID NO:111 to 125 (5′-UTR sub-sequences).

In a further embodiment the isolated polynucleotide comprises a sequence selected from SEQ ID NO: 111 to 125 (5′-UTR sub-sequences).

In a further embodiment the isolated polynucleotide comprises a sequence with at least 70% Identity to a sequence selected from SEQ ID NO:81 to 100 and 126 to 128 (whole 5′-UTR sequences).

In a further embodiment the isolated polynucleotide comprises a sequence selected from SEQ ID NO:81 to 100 and 126 to 128 (whole 5′-UTR sequences).

In a further embodiment the isolated polynucleotide comprises a sequence with at least 70% identity to a sequence selected from SEQ ID NO: 81 to 90, 93 to 97 and 126 to 128 (whole dicot 5′-UTR sequences).

In a further embodiment the isolated polynucleotide comprises a sequence selected from SEQ ID NO: 81 to 90, 93 to 97 and 126 to 128 (whole dicot 5′-UTR sequences).

In a further aspect the present disclosure provides an isolated polynucleotide with a sequence selected from SEQ ID NO 81 to 100 and 126 to 128 (whole 5′-UTR sequences) or a variant or fragment thereof.

In one embodiment the variant has at least 70% identity to a sequence selected from SEQ ID NO 81 to 100 and 126 to 128 (whole 5′-UTR sequences).

In a further aspect the present disclosure provides an isolated polynucleotide with a sequence selected from SEQ ID NO 111 to 125 (5′-UTR sub-sequences) or a variant or fragment thereof.

In one embodiment the variant has at least 70% identity to a sequence selected from SEQ ID NO 111 to 125 (5′-UTR sub-sequences).

In a further embodiment the variant or fragment comprises a sequence with at least 70% Identity to a sequence selected from SEQ ID NO 41 to 60 and 129 to 131 (uORF DNA sequences).

In a further embodiment the variant or fragment comprises a sequence selected from SEQ ID NO 41 to 60 and 129 to 131 (uORF DNA sequences).

In a further embodiment the variant or fragment comprises a sequence with at least 70% identity to a sequence selected from SEQ ID NO 41 to 50, 53 to 57 and 129 to 131 (dicot uORF DNA sequences).

In a further embodiment the variant or fragment comprises a sequence selected from SEQ ID NO 41 to 50, 53 to 57 and 129 to 131 (dicot uORF DNA sequences).

In a further embodiment the variant or fragment comprises a sequence with at least 70% identity to a sequence selected from SEQ ID NO:61 to 80 and 138 to 140 (conserved region of uORF DNA sequences).

In a further embodiment the variant or fragment comprises a sequence selected from SEQ ID NO:61 to 80 and 138 to 140 (conserved region of uORF DNA sequences).

In a further embodiment the variant or fragment comprises a sequence with at least 70% Identity to a sequence selected from SEQ ID NO: 61 to 70, 73 to 77 and 138 to 140 (conserved region of dicot uORF DNA sequences).

In a further embodiment the variant or fragment comprises a sequence selected from SEQ ID NO: 61 to 70, 73 to 77 and 138 to 140 (conserved region of dicot uORF DNA sequences).

In a further embodiment the variant encodes a sequence with at least 70% identity to at least one of SEQ ID NO:21 to 40 and 135 to 137 (conserved region of uORF peptides).

In a further embodiment the variant encodes a sequence selected from at least one of SEQ ID NO:21 to 40 and 135 to 137 (conserved region of uORF peptides).

In a further embodiment the variant encodes a sequence with at least 70% identity to at least one of SEQ ID NO: 21 to 30, 33 to 37 and 135 to 137 (conserved region of dicot uORF peptides).

In a further embodiment the variant encodes a sequence selected from at least one of SEQ ID NO: 21 to 30, 33 to 37 and 135 to 137 (conserved region of dicot uORF peptides).

In one embodiment the variant or fragment comprises a sequence with at least 70% identity to the amino sequence of SEQ ID NO: 108 (consensus motif uORF peptides).

In a further embodiment the variant or fragment comprises the amino sequence of SEQ ID NO: 108 (consensus motif uORF peptides).

In a further embodiment the variant encodes a sequence with at least 70% Identity to at least one of SEQ ID NO:1 to 20 and 132 to 134 (uORF peptides).

In a further embodiment the variant or fragment encodes a sequence selected from at least one of SEQ ID NO:1 to 20 and 132 to 134 (uORF peptides).

In a further embodiment the variant encodes a sequence with at least 70% identity to at least one of SEQ ID NO: 1 to 10. 13 to 17 and 132 to 134 (dicot uORF peptides).

In a further embodiment the variant or fragment encodes a sequence selected from at least one of SEQ ID NO: 1 to 10. 13 to 17 and 132 to 134 (dicot uORF peptides).

In one embodiment the isolated polynucleotide is modified.

In one embodiment, the modification is at least one of a deletion, an addition, or a substitution of at least one nucleotide in the sequence encoding the 5′-UTR.

In one embodiment the modification, reduced, disrupts, or prevents translation of a uORF polypeptide with the sequence of any one of SEQ ID NO: 1 to 20 and 132 to 134 (uORF peptides) or a variant thereof.

In a further embodiment the modification reduces, disrupts, or destroys the activity of a uORF polypeptide with the sequence of any one of SEQ ID NO: 1 to 20 and 132 to 134 (uORF peptides) or a variant thereof.

In one embodiment the variant comprises a sequence with at least 70% identity to any one of SEQ ID NO: 1 to 20 and 132 to 134 (uORF peptides).

In a further embodiment the variant comprises a sequence with at least 70% Identity to any one of SEQ ID NO: 1 to 10, 13 to 17 and 132 to 134 (dicot uORF peptides).

In a further embodiment the variant comprises a sequence with at least 70% identity to at least one of SEQ ID NO: 21 to 40 and 135 to 137 (uORF peptides conserved region).

In a further embodiment the variant comprises a sequence with at least one of SEQ ID NO: 21 to 40 and 135 to 137 (uORF peptides conserved region).

In a further embodiment the variant comprises a sequence with at least 70% identity to at least one of SEQ ID NO: 21 to 30, 33 to 37 and 135 to 137 (dicot uORF peptides conserved region).

In a further embodiment the variant comprises a sequence with at least one of SEQ ID NO: 21 to 30, 33 to 37 and 135 to 137 (dicot uORF peptides conserved region).

In one embodiment the variant or fragment comprises a sequence with at least 70% identity to the amino sequence of SEQ ID NO: 108 (consensus motif uORF peptides).

In a further embodiment the variant or fragment comprises the amino sequence of SEQ ID NO: 108 (consensus motif uORF peptides).

In one embodiment the polynucleotide, or variant, or fragment, is operably linked to a nucleic acid sequence of interest.

In a further embodiment the nucleic acid sequence of interest encodes a protein of interest.

In one embodiment the polynucleotide and nucleic acid sequence are not normally associated in nature.

When the polynucleotide is modified as discussed above, to disrupt expression or activity of the uORF polypeptide, the operably-linked sequence may be a GGP sequence. In this embodiment the modification removes repression, via the uORF, by ascorbate (which acts through a feedback loop). Expressing the GGP under the control of the modified polynucleotide may advantageously retain spatial and/or temporal expression of GGP similar to control by the native GGP promoter and 5′-UTR but stop the negative regulation of expression by ascorbate via the uORF polypeptide. In this embodiment the polynucleotide and nucleic acid sequence of interest may be normally associated in nature, except that the polynucleotide is in a modified form as discussed above.

### Polypeptides

In a further aspect the present disclosure provides an isolated polypeptide comprising a sequence selected from any one of SEQ ID NO: 1 to 20 and 132 to 134 (uORF peptides) or a variant or fragment thereof.

In one embodiment the variant or fragment comprises a sequence with at least 70% identity to a sequence selected from SEQ ID NO:21 to 40 and 135 to 137 (conserved region of uORF peptides).

In a further embodiment the variant or fragment comprises a sequence selected from SEQ ID NO:21 to 40 and 135 to 137 (conserved region of uORF peptides).

In one embodiment the variant or fragment comprises a sequence with at least 70% Identity to a sequence selected from SEQ ID NO: 21 to 30, 33 to 37 and 135 to 137 (conserved region of dicot uORF peptides).

In a further embodiment the variant or fragment comprises a sequence selected from SEQ ID NO: 21 to 30, 33 to 37 and 135 to 137 (conserved region of dicot uORF peptides).

In one embodiment the variant or fragment comprises a sequence with at least 70% identity to the amino sequence of SEQ ID NO: 108 (consensus motif uORF peptides).

In a further embodiment the variant or fragment comprises the amino sequence of SEQ ID NO: 108 (consensus motif uORF peptides).

In a further embodiment the variant comprises a sequence with at least 70% identity to a sequence selected from SEQ ID NO:1 to 20 and 132 to 134 (uORF peptides).

In a further embodiment the variant comprises a sequence with at least 70% identity to a sequence selected from SEQ ID NO: 1 to 10, 13 to 17 and 132 to 134 (dicot uORF peptides).

### Construct

In a further embodiment the present disclosure provides a construct comprising a polynucleotide of the present disclosure.

In one embodiment the polynucleotide is operably linked to a nucleic acid sequence of interest.

In a further embodiment the polynucleotide and nucleic acid sequence are not normally associated in nature.

In a further embodiment the nucleic acid sequence of interest encodes a protein of interest.

### Activity of Polynucleotides

In one embodiment the polynucleotide of the present disclosure is regulatable by a compound.

In this embodiment the expression of any nucleic acid sequence operably linked to the polynucleotide of the present disclosure is regulated by the compound.

In a preferred embodiment regulation is post-transcriptional.

Preferably expression of the polypeptide encoded by the operably linked nucleic acid is regulated by the compound.

In one embodiment, expression of the operably linked nucleic acid is regulated by interaction between the compound and the uORF peptide expressed by the polynucleotide of the present disclosure. In one embodiment interaction is direct. In a further embodiment the interaction is indirect. In a further embodiment the indirect interaction is via a further protein.

In one embodiment the compound is ascorbate, or a related metabolite. In a preferred embodiment the compound is ascorbate

When modified, as discussed above, the polynucleotide may no longer be regulatable by the compound. An application of this embodiment could be to express a GGP coding sequence under the control of the modified polynucleotide sequence. In this embodiment, the modification results in a reduction or removal of repression by the compound. When the compound is ascorbate, this results in a loss of a repression of GGP translation, and hence increased GGP production and increased ascorbate accumulation. In this embodiment the modification of the uORF, or uORF encoding sequence, may be in the context of the promoter and 5′-UTR sequence. Examples of the whole GGP promoter and 5-UTR sequence are provided in SEQ ID NO: 101 to 107 or a variant thereof. Use of the modified uORF in the context of the promoter and 5′-UTR sequence may retain some spatial or temporal expression of a native GGP sequence, but without repression of GGP translation by ascorbate via the uORF polypeptide.

### Cell

In a further embodiment the present disclosure provides a cell comprising a polynucleotide of the present disclosure, or a construct of the present disclosure.

Preferably the cell, or its precursor cell, has been genetically modified to comprise the polynucleotide of the present disclosure, or a construct of the present disclosure.

Preferably the cell, or its precursor cell, has been transformed to comprise the polynucleotide of the present disclosure, or a construct of the present disclosure.

### Plant Cells and Plants

In a further embodiment the present disclosure provides a plant cell or plant comprising a polynucleotide of the present disclosure or a construct of the present disclosure.

Preferably the plant cell or plant, or its precursor plant cell or plant, has been genetically modified to comprise the polynucleotide of the present disclosure, or a construct of the present disclosure.

Preferably the plant cell or plant, or its precursor plant cell or plant, has been transformed to comprise the polynucleotide of the present disclosure, or a construct of the present disclosure.

### Plant Part or Propagule

In a further embodiment the present disclosure provides a plant part or propagule comprising a polynucleotide of the present disclosure or a construct of the present disclosure.

Preferably the plant part or propagule, or its precursor plant cell or plant, has been genetically modified to comprise the polynucleotide of the present disclosure, or a construct of the present disclosure.

Preferably the plant part or propagule, or its precursor plant cell or plant, has been transformed to comprise the polynucleotide of the present disclosure, or a construct of the present disclosure.

In a further aspect the present disclosure provides a method for controlling or regulating expression of at least one nucleic acid sequence in a cell comprising transformation of the cell with a polynucleotide or construct of the present disclosure.

In a further aspect the present disclosure provides a method for controlling expression of at least one nucleic acid sequence in a plant cell or plant comprising transformation of the plant cell or plant with a polynucleotide or construct of the present disclosure.

In a further aspect the present disclosure provides a method for producing a cell with modified gene expression the method comprising transforming the cell with a polynucleotide or construct of the present disclosure.

In a further aspect the present disclosure provides a method for producing a plant cell or plant with modified gene expression the method comprising transforming plant cell or plant with a polynucleotide or construct of the present disclosure.

In a further aspect of the present disclosure provides a method for modifying the phenotype of a plant, the method including the stable incorporation into the genome of the plant, a polynucleotide or construct of the present disclosure.

Those skilled in the art will understand that introduction of the polynucleotide of the present disclosure into the cell, plant cell, or plant, may result in regulation or control of a nucleic acid sequence that is operably linked to the nucleic acid sequence before these sequences are introduced. In such an embodiment the polynucleotide of the present disclosure and operably linked nucleic acid of interest will be introduced together, for example on a construct of the present disclosure.

In an alternative embodiment, the polynucleotide of the present disclosure may be inserted into the genome, and control or regulate expression of a nucleic acid sequence, such as a protein encoding nucleic acid sequence, adjacent to the site of insertion.

In a preferred embodiment, the cell, plant cell or plant produces a compound that regulates or controls expression via the introduced polynucleotide of the present disclosure, or via a uORF polypeptide encoded by the introduced polynucleotide of the present disclosure.

Alternatively, the compound may be applied to the cell, plant cell or plant.

In a further aspect the present disclosure provides a plant cell or plant produced by a method of the present disclosure.

In one aspect the present disclosure provides a method for producing a plant cell or plant with increased ascorbate production, the method comprising modification of the 5′-UTR of a GGP gene in the plant cell or plant.

In one embodiment, the 5′-UTR is in the context of a polynucleotide sequence selected from any one of SEQ ID NO: 101 to 107 (GGP genomic sequences with promoter a 5′-UTR) or a variant thereof.

Preferably the variant has at least 70% Identity to the sequence of any one of SEQ ID NO: 101 to 107 (GGP genomic sequences with promoter a 5′-UTR).

In a further embodiment the 5′-UTR has a polynucleotide sequence selected from any one of SEQ ID NO: 81 to 100 and 126 to 128 (whole 5′-UTR sequences) or a variant thereof.

Preferably the variant has at least 70% Identity to the sequence of any one of SEQ ID NO: 81 to 100 and 126 to 128 (whole 5′-UTR sequences).

In a preferred embodiment the modification is in a uORF sequence in the 5′-UTR.

In a preferred embodiment the uORF has a sequence selected from any one of SEQ ID NO: 41 to 60 and 129 to 131 (uORF DNA sequences) or a variant thereof.

In a preferred embodiment the variant has at least 70% Identity to any one of SEQ ID NO: 41 to 60 and 129 to 131 (uORF DNA sequences).

In a preferred embodiment the variant has at least 70% identity to any one of SEQ ID NO: 41-50, 53-57 and 129 to 131 (dicot uORF DNA sequences).

In a further embodiment the variant comprises a sequence with at least 70% identity to any one of SEQ ID NO: 61-80 and 138 to 140 (conserved region of uORF DNA sequences).

In a further embodiment the variant comprises the sequence of any one of SEQ ID NO: 61-80 and 138 to 140 (conserved region of uORF DNA sequences).

In a further embodiment the variant comprises a sequence with at least 70% identity to any one of SEQ ID NO: 61-70, 73-77 and 138 to 140 (conserved region of dicot uORF DNA sequences).

In a further embodiment the variant comprises the sequence of any one of SEQ ID NO: 61-70, 73-77 and 138 to 140 (conserved region of dicot uORF DNA sequences).

In a further embodiment the uORF has the sequence of any one of SEQ ID NO: 1 to 20 and 132 to 134 (uORF polypeptide sequences) or a variant thereof.

In a further embodiment the variant has at least 70% identity the sequence of any one of SEQ ID NO: 1 to 20 and 132 to 134 (uORF polypeptide sequences).

In a further embodiment the variant has the sequence of any one of SEQ ID NO: 1 to 20 and 132 to 134 (uORF polypeptide sequences).

In a further embodiment the variant has at least 70% Identity the sequence of any one of SEQ ID NO: 1 to 10, 13 to 17 and 132 to 134 (dicot uORF polypeptide sequences).

In a further embodiment the variant has the sequence of any one of SEQ ID NO: 1 to 10, 13 to 17 and 132 to 134 (dicot uORF polypeptide sequences).

In a further embodiment the variant has at least 70% identity the sequence of any one of SEQ ID NO: 21 to 40 and 135 to 137 (conserved region of uORF polypeptide sequences).

In a further embodiment the variant has the sequence of any one of SEQ ID NO:21 to 40 and 135 to 137 (conserved region of uORF polypeptide sequences).

In a further embodiment the variant has at least 70% identity the sequence of any one of SEQ ID NO: 21 to 30, 33 to 37 and 135 to 137 (conserved region of dicot uORF polypeptide sequences).

In a further embodiment the variant has the sequence of any one of SEQ ID NO: 21 to 30, 33 to 37 and 135 to 137 (conserved region of dicot uORF polypeptide sequences).

In one embodiment the variant or fragment comprises a sequence with at least 70% identity to the amino sequence of SEQ ID NO: 108 (consensus motif uORF peptides).

In a further embodiment the variant or fragment comprises the amino sequence of SEQ ID NO: 108 (consensus motif uORF peptides).

### Modification

In one embodiment, the modification (also referred to as a mutation) is at least one of a deletion, an addition, or a substitution of at least one nucleotide in the sequence encoding the 5′-UTR.

In one embodiment the modification, reduced, disrupts, or prevents translation of a uORF polypeptide with the sequence of any one of SEQ ID NO: 1 to 20 and 132 to 134 (uORF peptides) or a variant thereof.

In a further embodiment the modification reduces, disrupts, or destroys the activity of a uORF polypeptide with the sequence of any one of SEQ ID NO: 1 to 20 and 132 to 134 (uORF peptides) or a variant thereof.

In one embodiment the variant comprises a sequence with at least 70% identity to any one of SEQ ID NO: 1 to 20 and 132 to 134 (uORF peptides).

In a further embodiment the variant comprises a sequence with at least 70% identity to any one of SEQ ID NO: 1 to 10, 13 to 17 and 132 to 134 (dicot uORF peptides).

In a further embodiment the variant comprises a sequence with at least 70% identity to at least one of SEQ ID NO: 21 to 40 and 135 to 137 (uORF peptides conserved region).

In a further embodiment the variant comprises a sequence with at least one of SEQ ID NO: 21 to 40 and 135 to 137 (uORF peptides conserved region).

In a further embodiment the variant comprises a sequence with at least 70% identity to at least one of SEQ ID NO: 21 to 30, 33 to 37 and 135 to 137 (dicot uORF peptides conserved region).

In a further embodiment the variant comprises a sequence with at least one of SEQ ID NO: 21 to 30, 33 to 37 and 135 to 137 (dicot uORF peptides conserved region).

In one embodiment the variant or fragment comprises a sequence with at least 70% identity to the amino sequence of SEQ ID NO: 108 (consensus motif uORF peptides).

In a further embodiment the variant or fragment comprises the amino sequence of SEQ ID NO: 108 (consensus motif uORF peptides).

In a further aspect the present disclosure provides a method for selecting a plant with increased ascorbate production, the method comprising testing of a plant for the presence of a first polymorphism in a polynucleotide of the present disclosure in the plant, or a further polymorphism linked to the first polymorphism.

In one embodiment presence of the first polymorphism, or the further polymorphism linked to the first polymorphism. Is indicative of at least one of a) to d).

In a further embodiment the further polymorphism is in linkage disequilibrium (LD) with the first polymorphism.

In a further embodiment the method includes the step of separating a selected plant from one of more non-selected plants.

In a further aspect the present disclosure provides a plant selected by the method of the present disclosure.

In a further aspect the present disclosure provides a group of plants selected by the method of the present disclosure. Preferably the group comprises at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 plants.

In a further aspect the present disclosure provides a method of producing ascorbate, the method comprising extracting ascorbate from a plant cell or plant of the present disclosure.

In a further aspect the present disclosure provides an antibody raised against a polypeptide of the present disclosure. In a further embodiment the present disclosure provides an antibody specific for a polypeptide of the present disclosure.

The polynucleotides, polypeptides, variants, and fragments, of the present disclosure may be derived from any species. The polynucleotides, polypeptides, variants, and fragments may be naturally occurring or non-naturally occurring. The polynucleotides, variants and fragments may be recombinantly produced and also may be the products of “gene shuffling’ approaches.

In one embodiment the polynucleotide, polypeptide, variant, or fragment, is derived from any plant species. The plant to be transformed or modified in the methods of the present disclosure may be from any plant species. The plant cells to be transformed or modified in the methods of the invention may be from any plant species.

In a further embodiment the plant is from a gymnosperm plant species.

In a further embodiment the plant is from an angiosperm plant species.

In a further embodiment the plant is from a from dicotyledonous plant species.

In a further embodiment the plant is from a fruit or nut species selected from a group comprising but not limited to the following genera: Citrus, Prunus, Actinidia, Malus, Citrus, Fragaria and Vaccinium.

Particularly preferred fruit plant species are: Citrus×sinensis, Citrus limon, Citrus×paradisi, Prunus dulcis, Prunus domestica, Prunus persica, Prunus avium, Actidinia deliciosa, A. chinensis, A. eriantha, A. arguta, hybrids of the four Actinidia species, Malus domestica and Malus sieboldii.

In a further embodiment the plant is selected from the group consisting of Actinidia eriantha, Cucumis sativus, Glycine max, Solanum lycopersicum, Vitis vinifera, Arabidopsis thaliana, Malus×domesticus, Medicago truncatula, Populus trichocarpa, Actinidia arguta, Actinidia chinensis, Fragaria vulgaris, Solanum tuberosum, and Zea mays.

In a further embodiment the plant is from a vegetable species selected from a group comprising but not limited to the following genera: Brassica, Lycopersicon and Solanum.

Particularly preferred vegetable plant species are: Lycopersicon esculentum and Solanum tuberosum.

In a further embodiment the plant is from monocotyledonous species.

In a further embodiment the plant is from a crop species selected from a group comprising but not limited to the following genera: Glycine, Zea, Triticum, Hordeum, Miscanthus, Saccharum, Beta and Oryza.

Particularly preferred crop plant species are: Hordeum vulgare, Miscanthus×giganteus, Saccharum officinarum, Triticum aestivum, Beta vulgaris, Oryza sativa, Glycine max, Cenchrus purpureus (synonym Pennisetum purpureum), and Zea mays.

In a further embodiment the plant is from a tree species grown for plantation forestry selected from a group comprising but not limited to the following genera: Eucalyptus, Salix, Betula, Populus, and Pinus

Particularly preferred plantation forestry species are: Pinus taeda and Eucalyptus grandis.

In a further embodiment the plant is selected from the group consisting of Actinidia eriantha, Cucumis sativus, Glycine max, Solanum lycopersicum, Vitis vinifera, Arabidopsis thaliana, Malus×domesticus, Medicago truncatula, Populus trichocarpa, Actinidia arguta, Actinidia chinensis, Fragaria vulgaris, Solanum tuberosum, and Zea mays.

### DETAILED DESCRIPTION

In this specification where reference has been made to patent specifications, other external documents, or other sources of information, this is generally for the purpose of providing a context for discussing the features of the present disclosure. Unless specifically stated otherwise, reference to such external documents is not to be construed as an admission that such documents, or such sources of information, in any jurisdiction, are prior art, or form part of the common general knowledge in the art.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides. Isolated and purified naturally occurring DNA or RNA sequences, synthetic RNA and DNA sequences, nucleic acid probes, primers and fragments.

Preferably the term “polynucleotide” Includes both the specified sequence and its compliment.

A “fragment” of a polynucleotide sequence provided herein is a subsequence of contiguous nucleotides, e.g., a sequence that is at least 15 nucleotides in length.

The fragments of the present disclosure comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the present disclosure.

The term “primer” refers to a short polynucleotide, usually having a free 3′OH group, that is hybridized to a template and used for priming polymerization of a polynucleotide complementary to the target.

### Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides of the present disclosure may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide. Preferably the fragment performs a function that is required for the biological activity and/or provides three-dimensional structure of the polypeptide.

The term “isolated” as applied to the polynucleotide or polypeptide sequences disclosed herein is used to refer to sequences that are removed from their natural cellular environment. In one embodiment the sequence is separated from its flanking sequences as found in nature. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the present disclosure being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Methods for Isolating or Producing Polynucleotides

The polynucleotide molecules of the present disclosure can be isolated by using a variety of techniques known to those of ordinary skill in the art. By way of example, such polypeptides can be isolated through use of the polymerase chain reaction (PCR) described in Mullis et al., Eds. 1994 The Polymerase Chain Reaction. Birkhauser, incorporated herein by reference. The polypeptides of the present disclosure can be amplified using primers, as defined herein, derived from the polynucleotide sequences of the present disclosure.

Further methods for isolating polynucleotides of the present disclosure include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the present disclosure may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods. 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transformed plant from a particular species, to transform such a plant with a sequence or sequences derived from that species. The benefit may be to alleviate public concerns regarding cross-species transformation in generating transformed organisms. Additionally, when down-regulation of a gene is the desired result, it may be necessary to utilize a sequence identical (or at least highly similar) to that in the plant, for which reduced expression is desired. For these reasons among others, it is desirable to be able to identify and isolate orthologs of a particular gene in several different plant species.

Variants (including orthologs) may be identified by the methods described.

### Methods for Identifying Variants

### Physical Methods

Variant polypeptides may be identified using PCR-based methods (Mullis et al., Eds. 1994 The Polymerase Chain Reaction. Birkhauser). Typically, the polynucleotide sequence of a primer, useful to amplify variants of polynucleotide molecules of the present disclosure by PCR, may be based on a sequence encoding a conserved region of the corresponding amino acid sequence.

Alternatively, library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the present disclosure (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

The variant sequences of the present disclosure, including both polynucleotide and polypeptide variants, may also be identified by computer-based methods well-known to those skilled in the art, using public domain sequence alignment algorithms and sequence similarity search tools to search sequence databases (public domain databases include GenBank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp colon slash slash file transfer protocol ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, Including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) Indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, BLASTN or tBLASTX algorithm.

Multiple sequence alignments of a group of related sequences can be carried out with CLUSTALW (Thompson, J. D., Higgins, D. G. and Gibson, T. J. (1994) CLUSTALW: Improving the sensitivity of progressive multiple sequence alignment through sequence weighting, positions-specific gap penalties and weight matrix choice. Nucleic Acids Research, 22:4673-4680, world wide web-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (worldwide web.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Isolating Polypeptides

The polypeptides of the present disclosure, including variant polypeptides, may be prepared using peptide synthesis methods well known in the art such as direct peptide synthesis using solid phase techniques (e.g., Stewart et al., 1969, in Solid-Phase Peptide Synthesis, WH Freeman Co, San Francisco Calif., or automated synthesis, for example using an Applied Biosystems 431A Peptide Synthesizer (Foster City, Calif.). Mutated forms of the polypeptides may also be produced during such syntheses.

The polypeptides and variant polypeptides of the present disclosure may also be purified from natural sources using a variety of techniques that are well known in the art (e.g., Deutscher, 1990, Ed, Methods in Enzymology, Vol. 182, Guide to Protein Purification,).

Alternatively, the polypeptides and variant polypeptides of the present disclosure may be expressed recombinantly in suitable host cells and separated from the cells as discussed below.

### Methods for Modifying Sequences

Methods for modifying the sequence of proteins, or the polynucleotide sequences encoding them, are well known to those skilled in the art. The sequence of a protein may be conveniently be modified by altering/modifying the sequence encoding the protein and expressing the modified protein. Approaches such as site-directed mutagenesis may be applied to modify existing polynucleotide sequences. Alternatively, restriction endonucleases may be used to excise parts of existing sequences. Altered polynucleotide sequences may also be conveniently synthesized in a modified form.

### Methods for Producing Constructs and Vectors

The genetic constructs of the present disclosure comprise one or more polynucleotide sequences of the present disclosure and/or polynucleotides encoding polypeptides of the present disclosure, and may be useful for transforming, for example, bacterial, fungal. Insect, mammalian, or plant organisms. The genetic constructs of the present disclosure are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Polynucleotides, Constructs or Vectors

The present disclosure provides a host cell which comprises a genetic construct or vector of the present disclosure. Host cells may be derived from, for example, bacterial, fungal, insect, mammalian, or plant organisms.

Host cells comprising genetic constructs, such as expression constructs, of the invention are useful in methods well known in the art (e.g., Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g., Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention. Plants comprising such cells also form an aspect of the invention.

Methods for transforming plant cells, plants, and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of plant transformation strategies are available (e.g., Birch, 1997, Ann Rev Plant Phys Plant Mol Biol, 48, 297, Hellens R P, et al (2000) Plant Mol Biol 42: 819-32, Hellens R et al (2005) Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Genetic constructs for expression of genes in transformed plants typically include promoters for driving the expression of one or more cloned polynucleotide, terminators, and selectable marker sequences to detest presence of the genetic construct in the transformed plant.

The promoters suitable for use in the constructs of this present disclosure are functional in a cell. tissue or organ of a monocot or dicot plant and include cell-, tissue- and organ-specific promoters, cell cycle specific promoters, temporal promoters. Inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the present disclosure. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal, e.g., luciferase (LUC), β-glucuronidase (GUS_, or green fluorescent protein (GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide of the present disclosure may include an antisense copy of a polynucleotide of the present disclosure. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene.

Genetic constructs designed for gene silencing may also include an inverted repeat. An ‘Inverted repeat’ is a sequence that is repeated where the second half of the repeat is in the complementary strand.

Genetic constructs designed for gene silencing may also include an inverted repeat. An ‘Inverted repeat’ is a sequence that is repeated where the second half of the repeat is in the complementary strand.

The transcript formed may undergo complementary base pairing to form a hairpin structure. Usually, a spacer of at least 3-5 bp between the repeated region is required to allow hairpin formation.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

The term genetic construct as used herein also includes small antisense RNAs and other such polypeptides effecting gene silencing.

Transformation with an expression construct, as herein defined, may also result in gene silencing through a process known as sense suppression (e.g., Napoli et al., 1990, Plant Cell 2, 279; de Carvalho Niebel et al., 1995, Plant Cell, 7, 347). In some cases, sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the expression of a polynucleotide of the invention is also contemplated.

The polynucleotide inserts in genetic constructs designed for gene silencing may correspond to coding sequence and/or non-coding sequence, such as promoter and/or intron and/or 5′ or 3′-UTR sequence, or the corresponding gene.

Other gene silencing strategies include dominant negative approaches and the use of ribozyme constructs (McIntyre, 1996, Transgenic Res, 5, 257).

Pre-transcriptional silencing may be brought about through mutation of the gene itself or its regulatory elements. Such mutations may include point mutations, frameshifts, insertions, deletions, and substitutions.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep, 17, 165); citrus plants (Pena et al., 1995, Plant Sci. 104, 183); caraway (Krens et al., 1997, Plant Cell Rep, 17, 39); banana (U.S. Pat. No. 5,792,935); soy bean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006: 25(2):117-23; Gonzalez Padilla et al., 2003 Plant Cell Rep. 22(1):38-45); strawberry (Oosumi et al., 2006 Planta. 223(6):1219-30; Folta et al., 2006 Planta April 14; PMID: 16614818), rose (Li et al., 2003), Rubus (Graham et al., 1995 Methods Mol Biol. 1995; 44:129-33), tomato (Dan et al., 2006, Plant Cell Reports V25:432-441), apple (Yao et al., 1995, Plant Cell Rep. 14, 407-412) and Actinidia eriantha (Wang et al., 2006, Plant Cell Rep. 25, 5: 425-31). Transformation of other species is also contemplated by the disclosure. Suitable methods and protocols are available in the scientific literature.

Several further methods known in the art may be employed to alter expression of a nucleotide and/or polypeptide of the disclosure. Such methods include but are not limited to Tilling (Till et al., 2003, Methods Mol Biol, 2%, 205), so called “Deletagene” technology (Li et al., 2001, Plant Journal 27(3), 235) and the use of artificial transcription factors such as synthetic zinc finger transcription factors. (e.g., Jouvenot et al., 2003, Gene Therapy 10, 513). Additionally, antibodies or fragments thereof, targeted to a particular polypeptide may also be expressed in plants to modulate the activity of that polypeptide (Jobling et al., 2003, Nat. Biotechnol. 21(1), 35). Transposon tagging approaches may also be applied. Additionally, peptides interacting with a polypeptide of the disclosure may be identified through technologies such as phase-display (Dyax Corporation). Such interacting peptides may be expressed in or applied to a plant to affect activity of a polypeptide of the disclosure. Use of each of the above approaches in alteration of expression of a nucleotide and/or polypeptide of the disclosure is specifically contemplated.

### Methods for Modifying Endogenous DNA Sequences in Plant

Methods for modifying endogenous genomic DNA sequences in plants are known to those skilled in the art. Such methods may involve the use of sequence-specific nucleases that generate targeted double-stranded DNA breaks in genes of interest. Examples of such methods for use in plants include: zinc finger nucleases (Curtin et al., 2011, Plant Physiol. 156:466-473.; Sander, et al., 2011, Nat. Methods 8:67-69.), transcription activator-like effector nucleases or “TALENs” (Cermak et al., 2011, Nucleic Acids Res. 39:e82; Mahfouz et al., 2011 Proc. Natl. Acad. Sci. USA 108:2623-2628; Li et al., 2012 Nat. Biotechnol. 30:390-392), and LAGLIDADG homing endonucleases, also termed “meganucleases” (Tzfira et al., 2012. Plant Biotechnol. J. 10:373-389).

A preferred method of practicing the invention is to use genome editing to produce a “targeted genetic modification” as referenced herein. The terms “genome editing”, “genome edited”, “genome modified”, “genetically modified” are used interchangeably to describe plants with specific DNA sequence changes in their genomes wherein those DNA sequence changes include changes of specific nucleotides, the deletion of specific nucleotide sequences or the insertion of specific nucleotide sequences.

As used herein, a technique for introducing a “targeted genetic modification” refers to any method, protocol, or technique that allows the precise and/or targeted editing at a specific location (also referred to a “locus” or “native locus” in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. CRISPR is an acronym for clustered, regularly interspaced, short, palindromic repeats and Cas an abbreviation for CRISPR-associated protein: for a review, see Khandagal and Nadal, Plant Biotechnol. Rep., 2016, 10, 327. Engineered meganucleases, zinc finger nucleases (ZFN), transcription activator-like effector nucleases (TALENs) can also be used. US Patent Application 2016/0032297 provides detailed methodology for these methods. Another gene editing methodology that can be applied uses so-called ARCUS nucleases which leverage the properties of a naturally occurring gene editing enzyme—the homing endonuclease I-CreI—which evolved in nature to make a single, highly specific DNA edit before using its built-in safety switch to shut itself off.

Genome editing tools can accurately change the architecture of a genome at specific target locations. These tools can be efficiently used for the generation of plants with high crop yields, desired alterations in composition, and resistance to biotic and abiotic stresses. It may be challenging to achieve all desired modifications using a particular genome editing tool. Thus, multiple genome editing tools have been developed to facilitate efficient genome editing. Some of the major genome editing tools used to edit plant genomes are: Homologous recombination (HR), zinc finger nucleases (ZFNs), transcription activator-like effector nucleases (TALENs), pentatricopeptide repeat proteins (PPRs), the CRISPR/Cas9 system. RNA interference (RNAi), cisgenesis, and intragenesis. In addition, site-directed sequence editing and oligonucleotide-directed mutagenesis have the potential to edit the genome at the single-nucleotide level. Recently, adenine base editors (ABEs) have been developed to mutate A-T base pairs to G-C base pairs. ABEs use deoxyadeninedeaminase (TadA) with catalytically impaired Cas9 nickase to mutate A-T base pairs to G-C base pairs. A summary of these methods an applicability is provided by Mohanta et al., Genes (Basel). 2017 December; 8(12): 399.

Such genome editing methods encompass a wide range of approaches to precisely remove genes. gene fragments, to alter the DNA sequence of coding sequences or control sequences, or to insert new DNA sequences into genes or protein coding regions to reduce or increase the expression of target genes in plant genomes (Belhaj, K. 2013, Plant Methods, 9, 39; Khandagale and Nadal, 2016, Plant Biotechnol Rep, 10, 327). Preferred methods involve the in vivo site-specific cleavage to achieve double stranded breaks in the genomic DNA of the plant genome at a specific DNA sequence using nuclease enzymes and the host plant DNA repair system. Multiple approaches are available for producing double stranded breaks in genomic DNA, and thus achieve genome editing, including the use of the CRISPR/Cas system.

An extensive overview of the CRISPR/Cas system and useful applications thereof can be found at www.addgene.org/guides/crispr/

The CRISPR/Cas genome editing system provides flexibility in targeting specific sequences for modification within the genome and enables the execution of a range of different edits including the activation or upregulation of target loci or the knock-out of target loci. The method relies on providing the Cas enzyme and a short guide RNA “gRNA” containing a short guide sequence (˜20 bp), with sequence complementarity to the target DNA sequence in the plant genome. Depending on the type of Cas enzyme, alternatively a DNA, an RNA/DNA hybrid, or a double stranded DNA guide polynucleotide can be used. The guide portion of this guide polynucleotide directs the Cas enzyme to the desired cut site for cleavage with a recognition sequence for binding the Cas enzyme.

The target in the plant genome can be any ˜20 nucleotide DNA sequence, provided that the sequence is unique compared to the rest of the genome and also that the target is present immediately adjacent to a Protospacer Adjacent Motif (PAM). The PAM sequence serves as a binding signal for Cas9, but the exact sequence depends on which Cas protein is being used. A list of Cas proteins and PAM sequences can be found at www.addgene.org/guides/crispr/#pam-table

The simplest application of CRISPR/Cas is to produce knockout or loss of function alleles in a target locus. The gRNA targets the Cas enzyme to a specific locus in the genome, which then produces a double stranded break. The resulting DSB is then repaired by one of the general repair pathways present in the cell. This typically causes small nucleotide insertions or deletions (indels) at the DSB site. In most cases, small indels in the target DNA result in amino acid deletions, insertions, or frameshift mutations leading to premature stop codons within the open reading frame (ORF) of the targeted gene. The ideal result is a loss-of-function mutation within the targeted gene. However, the strength of the knockout phenotype for a given mutant cell must be validated experimentally, for example for testing for the presence of transcript from the target ORF by RT-PCR or hybridization-based approaches. These features make the CRISPR/Cas system a suitable tool for knockout of uORFs.

CRISPR/Cas can also be used to produce more sophisticated changes to the native sequence at targeted loci in the genome. This can involve inserting sequences, replacing sequences, or editing specific bases so as to insert or create new domains within a polypeptide encoded at a desired locus. One way to introduce such changes is to make use of the high fidelity but low efficiency high fidelity homology directed (HDR) repair pathway within the cell. In order to make such precise modifications using HDR, a DNA repair template incorporating the desired genome modification that the practitioner desires to create at the target locus must be delivered into the cell type of interest with the gRNA(s) and Cas9 or Cas9 nickase. The repair template must contain the desired edit as well as additional homologous sequence immediately upstream and downstream of the target (termed left and right homology arms). The length of each homology arm is dependent on the size of the change being introduced, with larger insertions requiring longer homology arms. Since the efficiency of Cas9 cleavage is relatively high and the efficiency of HDR is relatively low, a large portion of the Cas9-induced DSBs will be repaired to produce edits not comprising the specific desired change. Thus, an additional confirmation/screening step is required to select one of more cells from the edited population that contain the desired change. These cells then can be regenerated into a population of cells, a tissue, organ or whole plant or plant population. Such selection can be achieved by incorporating a marker sequence into the edit, which is readily screened or by PCR or hybridization-based methods.

CRISPR-related gene editing systems can also be deployed to change specific bases without the need for double stranded breaks. Such approaches are referred to in the art as “base editing” systems. Base editing enables the irreversible conversion of a specific DNA base into another at a targeted genomic locus, for example conversing C to T, or A to G. Unlike other genome-editing tools, base editing can be achieved without double-strand breaks. When introducing a point mutation at a target locus, base editing is more efficient than traditional genome editing techniques. Since many genetic diseases arise from point mutations, base editing has important applications in disease research. Using these systems, the skilled practitioner can create a targeted genetic modification comprising an amino acid substitution or the creation of start or stop codon.

To avoid relying on HDR, which has low efficiency, researchers have developed two classes of base editors: cytosine base editors (CBEs) and adenine base editors (ABEs). Cytosine base editors are created by fusing Cas9 nickase or catalytically inactive “dead” Cas9 (dCas9) to a cytidine deaminase like APOBEC. As with traditional CRISPR techniques, base editors are targeted to a specific locus by a gRNA, and they can convert cytidine to uridine within a small editing window near the PAM site. Uridine is subsequently converted to thymidine through base excision repair, creating a C to T change. Likewise, adenosine base editors have been engineered to convert adenosine to inosine, which is treated like guanosine by the cell, creating an A to G change.

Adenine DNA deaminases do not exist in nature, but these enyzmes have been created by directed evolution of the Escherichia coli TadA, a tRNA adenine deaminase. Like cytosine base editors, the evolved TadA domain is fused to a Cas9 protein to create the adenine base editor. Both types of base editors are available with multiple Cas9 variants including high fidelity Cas9′s. Further advancements have been made by optimizing expression of the fusions, modifying the linker region between Cas variant and deaminase to adjust the editing window, or adding fusions that increase product purity such as the DNA glycosy lase inhibitor (UGI) or the bacteriophage Mu-derived Gam protein (Mu-GAM).

While many base editors are designed to work in a very narrow window proximal to the PAM sequence, some base editing systems create a wide spectrum of single-nucleotide variants (somatic hypermutation) in a wider editing window, and are thus well suited to directed evolution applications. Examples of these base editing systems include targeted AID-mediated mutagenesis (TAM) and CRISPR-X, in which Cas9 is fused to activation-induced cytidine deaminase (AID).

Other CRISPR systems, specifically the Type VI CRISPR enzymes Cas13a/C2c2 and Cas13b, target RNA rather than DNA. Fusing a hyperactive adenosine deaminase that acts on RNA, ADAR2(E488Q), to catalytically dead Cas13b creates a programmable RNA base editor that converts adenosine to inosine in RNA (termed REPAIR). Since inosine is functionally equivalent to guanosine, the result is an A->G change in RNA. The catalytically inactive Cas13b ortholog from Prevotella sp., dPspCas13b, does not appear to require a specific sequence adjacent to the RNA target, making this a very flexible editing system. Editors based on a second ADAR variant, ADAR2 (E488Q/T375G), display improved specificity, and editors carrying the delta-984-1090 ADAR truncation retain RNA editing capabilities and are small enough to be packaged in AAV particles.

In the context of this description, it is recognized that the term Cas nuclease includes any nuclease which site-specifically recognizes CRISPR sequences based on gRNA or DNA sequences and includes Cas9, Cpfl and others described below. Many authors have identified that CRISPR/Cas genome editing, is a preferred way to edit the genomes of complex organisms (Sander and Joung, 2013, Nat Biotech, 2014, 32, 347; Wright et al., 2016, Cell. 164, 29) including plants (Zhang et al., 2016, Journal of Genetics and Genomics, 43, 151; Puchta 2016, Plant J., 87, 5; Khandagale and Nadaf, 2016, Plant Biotechnol. Rep., 10, 327). US Patent Application 2016/020822 provides extensive description of the materials and methods useful for genome editing in plants using the CRISPR/Cas9 system and describes many of the uses of the CRISPR/Cas9 system for genome editing of a range of gene targets in crops.

It is further recognized that many variations of the CRISPR/Cas system can be used for applying the invention herein, including the use of wild-type Cas9 from Streptococcus pyogenes (Type II Cas) (Barakate and Stephens, 2016, Frontiers in Plant Science, 7, 765; Bortesi and Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187). Other examples include Tru-gRNA/Cas9 in which off-target mutations are significantly decreased (Fu et al., 2014, Nature biotechnology, 32, 279; Osakabe et al., 2016, Scientific Reports, 6, 26685; Smith et al., 2016, Genome biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84). Further variations comprise the Type I and Type III systems in which multiple Cas proteins are expressed to achieve editing (Li et al., 2016, Nucleic acids research, 44:e34; Luo et al., 2015, Nucleic acids research, 43, 674), the Type V Cas system using the Cpfl enzyme (Kim et al., 2016, Nature biotechnology, 34, 863; Toth et al., 2016, Biology Direct, 11, 46; Zetsche et al., 2015, Cell, 163, 759), DNA-guided editing using the NgAgo Argonaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819). A unique nuclease Cpfl, an alternative to Cas9 has advantages over the Cas9 system in reducing off-target edits which creates unwanted mutations in the host genome. Examples of crop genome editing using the CRISPR/Cpfl system include rice (Tang et, al., 2017, Nature Plants 3, 1-5; Wu et, al., 2017, Molecular Plant, Mar. 16, 2017) and soy bean (Kim et., al., 2017, Nat Commun, 8, 14406). Other authors have described the use of Argonaute related proteins as an alternative to CRISPR systems for gene editing (Hegge et al. Nature Rev. Microbiol. 2017. Epub 2017 Jul. 25. pmid:28736447; Swarts et al. Nucleic Acids Res., 2015:43(10):5120-9. Epub 2015 May 1. pmid:25925567; Swarts et al. Nature. 2014:507(7491):258-61. Epub 2014 Feb. 18. pmid:24531762. See also PCT Application Number PCT/US2019/025163 and/or Publication Number WO2019204266A1.

Detailed methodologies for gene editing in plants to create new crop traits, including the selection of cells containing the desired edits, and methods for introducing the CRISPR system components into an initial target plant cell are set forth in published patent application WO2019195157. As specified therein. the “guide polynucleotide” in a CRISPR system also relates to a polynucleotide sequence that can form a complex with a Cas endonuclease and enables the Cas endonuclease to recognize and optionally cleave a DNA target site. The guide polynucleotide can be a single molecule (i.e., a single guide RNA (gRNA) that is a synthetic fusion between a crRNA and part of the tracrRNA sequence) or two molecules (i.e., the crRNA and tracrRNA as found in natural Cas9 systems in bacteria). The guide polynucleotide sequence can be provided as an RNA sequence or can be transcribed from a DNA sequence to produce an RNA sequence. The guide polynucleotide sequence can also be provided as a combination RNA-DNA sequence (see for example, Yin, H. et al., 2018, Nature Chemical Biology, 14, 311). As used herein “guide RNA” sequences comprise a variable targeting domain, called the “guide”, complementary to the target site in the genome, and an RNA sequence that interacts with the Cas9 or Cpfl endonuclease, called the “guide RNA scaffold”. A guide polynucleotide that solely comprises ribonucleic acids is also referred to as a “guide RNA”. As used herein the “guide target sequence” refers to the sequence of the genomic DNA adjacent to a PAM site, where the gRNA will bind to cleave the DNA. The “guide target sequence” is often complementary to the “guide” portion of the gRNA, however several mismatches, depending on their position, can be tolerated and still allow Cas mediated cleavage of the DNA. The method also provides introducing single guide RNAs (gRNAs) into plants. The single guide RNAs (gRNAs) include nucleotide sequences that are complementary to the target chromosomal DNA. The gRNAs can be, for example, engineered single chain guide RNAs that comprise a crRNA sequence (complementary to the target DNA sequence) and a common tracrRNA sequence, or as crRNA-tracrRNA hybrids. The gRNAs can be introduced into the cell or the organism as a DNA with an appropriate promoter, as an in vitro transcribed RNA, or as a synthesized RNA. Basic guidelines for designing the guide RNAs for any target gene of interest are well known in the art as described for example by Brazelton et al. (Brazelton, V.A. et al., 2015, GM Crops & Food, 6, 266-276) and Zhu (Zhu, L. J. 2015, Frontiers in Biology, 10, 289-296).

Published patent applications WO2019195157 and WO2019204266A1 also provides example of the types of mutation that can lead to increased activity of transcription factor polypeptides. These include mutations to the coding sequence that give rise to amino acid changes in the encoded protein.

In certain preferred embodiments of the present invention, the guide polynucleotide/Cas endonuclease system can be used to allow for the insertion of a promoter or promoter element, such as an enhancer element, of any one the transcription factor sequences of the invention, wherein the promoter insertion (or promoter element deletion) results in any one of the following or any one combination of the following: a permanently activated gene locus, an increased promoter activity (increased promoter strength), an increased promoter tissue specificity, a decreased promoter tissue specificity, a new promoter activity, an extended window of gene expression, a modification of the timing or developmental progress of gene expression, a mutation of DNA binding elements and/or an addition of DNA binding elements.

The guide RNA/Cas endonuclease system can be used to allow for the insertion of a promoter element to increase the expression of the transcription factor sequences of the invention. Promoter elements, such as enhancer elements, are often introduced in promoters driving gene expression cassettes in multiple copies for trait gene testing or to produce transgenic plants expressing specific traits. Enhancer elements can be, but are not limited to, a 35S enhancer element (Benfey et al, EMBO J., 1989; 8: 2195-2202). In some plants (events), the enhancer elements can cause a desirable phenotype, a yield increase, or a change in expression pattern of the trait of interest that is desired. It may be desired to remove the extra copies of the enhancer element while keeping the trait gene cassettes intact at their integrated genomic location. The guide RNA/Cas endonuclease can be used to remove the unwanted enhancing element from the plant genome. A guide RNA can be designed to contain a variable targeting region targeting a target site sequence of 12-30 bps adjacent to a NGG (PAM) in the enhancer. The Cas endonuclease can make cleavage to insert one or multiple enhancers.

To repress the function of a target uORF and activate the downstream mORF, bases can be deleted from the uORF, or additional stop codons can be created. Other mutations or edits that may be used to repress the function of a target uORF include mutations of the start ATG codon, amino acid deletions, insertions, or frameshift mutations leading to premature stop codons or any of a number of deleterious mutations within the uORF. In some cases, it may be optimum to substitute bases within a uORF to produce an optimized phenotype whereby an increased yield, increased stress tolerance, or altered biochemical composition may be obtained without substantial off types such as, for example, organ abnormalities or dwarfing.

Delivery of gene editing components into plant cells and plants:

Sandhya et al. 2020. J. Genet. Eng. Biotechnol. 18: 25. Published online 2020 Jul. 7. doi: 10.1186/s43141-020-00036-8, present methods for delivering gene editing tools such CRISPR/Cas9 components into plants to execute the gene editing process. The effective delivery of CRISPR/Cas9 components, including the guide sequence, the CAS9, and where applicable a DNA-repair template containing the desired sequence edit, into plant cells is critical for editing to be efficient. The practitioner can select from a variety of delivery methods to introduce the gene editing components into plant cells. These include Agrobacterium-mediated transformation, bombardment or biolistic methods of transformation, floral-dip, and PEG-mediated protoplast transformation. Additional methods include nanoparticle and pollen magnetofection-mediated delivery systems (Kwak et ai., 2019, Nature Nanotechnology, DOI 10.1038/S41565-019-0375-4) (Demirer et al, 2019, Nature Nanotechnology, DOI 10.1038/S41565-019-0382-5) can be used. CRISPR constructs can be coated onto gold particles for gene gun mediated introduction into plant cells. CRISPR constructs can be transfected into protoplasts using PEG, or introduced via an Agrobacterium strain harboring a CRISPR vector. Components may also be introduced via floral dip (Castel et al., 2019, PLoS One 14:e0204778) or a pollen-tube tube pathway-based method. In the next step, a plant or plant cell containing a targeted genetic modification produced by the introduced CRISPR system is selected. This may involve regenerating a cell containing the modification into an explant, a plant tissue, or whole plant. In some instances, this procedure involves selecting explants harboring the genome edit on selection plates and regenerating a whole plant. Finally, PCR and Sanger sequencing are generally used for confirmation that the desired sequence edit has been successfully introduced into the selected plant. The selected plant is then examined to confirm that it exhibits the target trait of interest that was initially sought by introducing the genome modification.

Sandhya et al. 2020. J. Genet. Eng. Biotechnol. 18: 25. Published online 2020 Jul. 7. doi: 10.1186/s43141-020-00036-8, present tables showing which methods can be successfully applied to particular crops. For example, the following plants can all be successfully gene edited using PEG mediated delivery of CRISPR system components: Apple, Brassica oleracea, Brassica rapa, Citrullus lanatus, Glycine max, Grapevine, Oryza sativa, Petunia, Physcomitrella patens, Solanum lycopersicum, Triticum aestivum, and Zea mays. By way of further example, the following plants can all be successfully gene edited using particle bombardment mediated delivery of CRISPR system components: Glycine max, Hordeum vulgare, Oryza sativa, Triticum aestivum and Zea mays. By way of yet further example, the following plants can all be successfully gene edited using particle bombardment mediated delivery of CRISPR system components: Arabidopsis thaliana, Banana, Citrus sinensis, Cucumis sativum, Glycine max, Kiwi fruit, Lotus japonicus, Marchantia polymorpha, Medicago truncatula, Nicotiana benthamaina, Nicotiana tabacum, Oryza sativa, Populus, Salvia miltiorrhiza, Solanum lycopersicum, Solanum lycopersicum, Sorghum bicolor, Triticum aestivum, and Zea mays.

In certain embodiments of the disclosure, one of the above technologies (e.g., TALENs or a Zinc finger nuclease) can be used to modify one or more base pairs in the uORF in order to disable it, so it is no longer translatable.

In one embodiment the first base pair of the ACG start codon is changed to TCG to accomplish this. This would inactivate the ascorbate feedback regulation of GGP translation and allow increases of ascorbate concentration in the plant.

Alternatively, a codon for a highly conserved amino acid in the uORF can be changed to stop the uORF from functioning in down regulating translation of the GGP at high ascorbate. For example, a His residue in the conserved region of the uORF can be changed to a Leu.

In a further embodiment an early base pair in the uORF is altered to introduce a stop codon, and cause early termination of the uORF which stops ascorbate feedback regulation of the translation of GGP.

Those skilled in the art will thus appreciate that there are numerous ways in which the uORF can be disrupted to remove negative regulation by ascorbate and to increase ascorbate production. Any such method is included within the scope of the disclosure.

### Plants

The term “plant” is intended to include a whole plant, any part of a plant, propagules, and progeny of a plant.

The term ‘propagule’ means any part of a plant that may be used in reproduction or propagation, either sexual or asexual, including seeds and cuttings.

The plants of the disclosure may be grown and either self-ed or crossed with a different plant strain and the resulting off-spring from two or more generations also form an aspect of the present disclosure, provided they maintain the transgene or modification of the invention.

This disclosure may also be said broadly to consist in the parts, elements and features referred to or indicated in the specification of the application. Individually or collectively, and any or all combinations of any two or more said parts, elements or features, and where specific integers are mentioned herein which have known equivalents in the art to which this disclosure relates, such known equivalents are deemed to be incorporated herein as if individually set forth.

### EXAMPLES

The specification, now being generally described, will be more readily understood by reference to the following Examples, which are included merely for purposes of illustration of certain aspects and embodiments of the present disclosure and are not intended to limit the present disclosure. It is not the intention to limit the scope of the invention to the Examples only. As would be appreciated by a skilled person in the art, many variations are possible without departing from the scope of the disclosure.

### Example 1. Elucidation of the Control of GGP Expression

### Summary

Ascorbate (vitamin C) Is an essential antioxidant and enzyme co-factor in both plants and animals. Ascorbate concentration is tightly regulated in plants, partly to respond to abiotic or biotic stress. Applicants have shown that ascorbate levels are controlled via the post-transcriptional repression of GDP-L-galactose phosphory lase (GGP), the rate-limiting enzyme in the ascorbate biosynthesis pathway, This regulation requires the translation of a Cis-acting uORF (upstream open reading frame), which initiates from a non-canonical start codon and represses the translation of the downstream GGP ORF under high ascorbate. Removal of this uORF allows plants to produce high levels of ascorbate. The uORF is present in the GGP gene from both lower and higher plants indicating it is an ancient mechanism to control ascorbate levels.

Ascorbate (vitamin C) is an essential biochemical found in most living organisms with a central role of controlling the redox potential of the cell (Asensi-Fabado et al., 2010, Trends Plant Sci. 15, 582; Foyer et al., Plant Physiol. 155, 2 (2011)) as well as serving as an enzyme cofactor (Mandl et al., 2009, Br. J. Pharmacol. 157, 1097). Ascorbate concentrations are regulated according to demand; for example, leaf ascorbate concentrations increase under high light intensities when the need for ascorbate is greatest (Bartoli et al., 2006, J. Exp. Bot. 57, 1621; Gatzek et al., 2002, Plant J. 30, 541). However, the mechanism by which ascorbate biosynthesis is regulated is not known. Applicants have shown previously that the enzyme GDP L-Galactose phosphory lase (GGP) is central to determining ascorbate in plants (Bulley et al., 2012, Plant Biotechnol J 10, 390; Bulley et al., 2009, J. Exp. Bot. 60, 765), suggesting it may serve a regulatory role.

### Results and Discussion

To investigate if the GGP gene is regulated by ascorbate levels. Applicants fused the kiwifruit GGP promoter with its 5′-UTR (SEQ ID NO: 101) to the luciferase (LUC) reporter gene and expressed the construct transiently in Nicotiana benthamiana leaves. (Hellens et al., 2005, Plant Methods 1, 13.). Applicants manipulated ascorbate by also expressing just the coding sequence of GGP under a strong constitutive promoter. A doubling of ascorbate concentration from ˜2 mM (20 mg/100 g FW) to 4 mM was sufficient to reduce the relative LUC activity by 50%, and when ascorbate was increased close to 10 mM, >90% of LUC activity was abolished. Similarly, the Arabidopsis GGP (VTC2; At4g26850) promoter and 5′-UTR (SEQ ID NO: 102) also conferred ascorbate dependent repression on a LUC reporter gene.

In contrast, high ascorbate had no effect on relative LUC activity using a control promoter for a gene unrelated to ascorbate metabolism (T-8: an Arabidopsis bHLH transcription factor controlling polyphenolic biosynthesis). Additional controls demonstrate that this regulation was specific to GGP sequences, independent of the level of expression of the transgenes and was reflected in LUC protein changes (Tables 2-4).

TABLE 2
The absolute value of LUC or REN does not significantly influence the LUC/REN
ratio. The relationship between LUC and REN was linear over a 200-fold
range. The subscript P refers to the whole promoter including any 5′-UTR
from that gene. Other details are found in the methods section.

| | | Slope of | | Mean LUC | Mean REN |
|---|---|---|---|---|---|
| | Agrobacterium | relationship | | Luminescent | Luminescent |
| | culture | between | | units +/− | units +/− |
| | dilution | LUC and | | Standard | Standard |
| Construct | as a % | REN | LUC/REN | Error | Error |
| GGP_{P} | 100 | 0.07 | 0.11 | 7442 .+−. 785 | 67908 .+−. 7429 |
| GGP_{P} | 10 | 0.12 | 0.11 | 2777 .+−. 277 | 25001 .+−. 1684 |
| TT8_{P} | 100 | 2.66 | 2.96 | 122573 .+−. 15748 | 41478 .+−. 4557 |
| TT8_{P} | 10 | 2.64 | 2.91 | 25560 .+−. 2255 | 8786 .+−. 710 |
| TT8_{P} | 8 | 2.97 | 2.69 | 23621 .+−. 2672 | 8785 .+−. 552 |
| TT8_{P} | 5 | 1.56 | 2.24 | 10176 .+−. 547 | 4537 .+−. 210 |
| TT8_{P} | 3 | 1.72 | 2.4 | 6412 .+−. 450 | 2676 .+−. 172 |
| TT8_{P} | 1 | 2.39 | 2.49 | 1739 .+−. 274 | 699 .+−. 101 |
| TT8_{P} | 0.5 | 2.53 | 2.79 | 1482 .+−. 175 | 531 .+−. 43 |

TABLE 3
Comparison of the effect of ascorbate on a range of control genes

| | Ascorbate | | | | | Slope |
|---|---|---|---|---|---|---|
| | mg/100 g | | Std | | | of LUC |
| Treatment | FW | LUC/REN | error | N | | REN |
| TT8_{P} | 22 | 1.95 | 0.24 | 6 | a | 1.89 |
| TT8_{P} + GGP | 78 | 4.09 | 0.28 | 12 | b | 4.71 |
| EF1a_{P} | 23 | 0.767 | 0.086 | 6 | a | 0.78 |
| ET1a_{P} + GGP | 82 | 0.733 | 0.065 | 12 | a | 0.775 |
| ACt2_{P} | 24 | 0.385 | 0.042 | 6 | a | 0.327 |
| ACt2_{P} + GGP | 82 | 0.55 | 0.074 | 12 | a | 0.586 |
| ACt7_{P} | 22 | 0.815 | 0.037 | 6 | a | 0.757 |
| ACt7_{P} + GGP | 78 | 0.843 | 0.036 | 12 | a | 0.834 |

+ GGP refers to the cotransformation of the CDS of the GGP from kiwifruit (GenBank accession FG528585) under the control of the 35S promoter in order to raise ascorbate. The slope of LUC REN is the slope of the plot of LUC values against REN values forced through the origin as a comparison to the LUC/REN ratios. N is the number of independent LUC/REN ratios measured. The subscript P refers to the whole promoter including any 51-UTR from that gene. Within a block of two rows. LUC/REN means with the same letter do not significantly differ at p < 0.01. Other details are found in the methods section.

TABLE 4
Effect of adding a control gene to the gene
used to manipulate ascorbate concentration.

| | | | | | | |
|---|---|---|---|---|---|---|
| GGP_{P} | 22 | 0.251 | 0.027 | 12 | a | 0.23 |
| GGP_{P} + GGP | 65 | 0.028 | 0.008 | 12 | b | 0.022 |
| GGP_{P} + FG429343 | 22 | 0.226 | 0.044 | 12 | a | 0.183 |
| TT8_{P} | 25 | 3.87 | 0.44 | 12 | a | 3.12 |
| TT8_{P} + GGP | 50 | 2.42 | 0.21 | 12 | b | 2.25 |
| TT8_{P} + FG429343 | 25 | 2.73 | 0.16 | 12 | b | 3.06 |

To check that expression of an extra gene (GGP) to increase ascorbate did not directly affect the LUC/REN ratio, we substituted another control gene, GenBank accession FG429343, an Actinidia deliciosa methyl transferase which had no direct effect of the ascorbate concentration. The subscript P refers to the whole promoter. Within a block of three rows, LUC/REN means with the same letter do not significantly differ at p < 0.01. Other details are found in the methods section.

In the experiments described, leaf ascorbate was manipulated through changes in expression of the GGP coding sequence. In order to separate the effects of ascorbate from possible effect of the GGP protein (Muller-Moule, P., 2008, Plant Mol. Biol. 68, 31). Applicants expressed both GGP and GME separately and together. Applicants have previously shown (Bulley et al., 2009, J. Exp. Bot. 60, 765.) that GGP expressed alone in tobacco has a moderate effect, GME very little effect, whereas when expressed together there is a strong synergistic stimulation of ascorbate concentration. Thus, by varying the ratios of these two genes, ascorbate can be manipulated independently of the amount of GGP protein. The response of the ratio to ascorbate followed a smooth curve in spite of different levels of GGP protein associated with different ascorbate concentrations, showing ascorbate or a related metabolite is the factor reducing the LUC activity.

To test whether the effect of ascorbate was mediated by the untranscribed promoter or the 5′-UTR Applicants undertook two experiments. Firstly. Applicants swapped the 5′-UTR regions between the GGP TT8 promoters. We transiently expressed these in leaves and measured the relative LUC activity. Increased ascorbate reduced the LUC activity only when the 5′-UTR from GGP was present (Table 5). Secondly, Applicants deleted two regions within the 5′-UTR that were especially strongly conserved at the DNA level between species. The first was from ˜387 to ˜432 bp and the second between ˜514 b and ˜597 bp with the rest of the GGP promoter intact, and tested them using the reporter assay. All deletions caused the loss of the ability of ascorbate to down regulate the reporter gene expression. These experiments show that the 5′-UTR is necessary and sufficient for down regulation by ascorbate.

TABLE 5

| | Ascorbate | | | | | Slope |
|---|---|---|---|---|---|---|
| | mg/100 g | LUC/ | Std | | | of LUC |
| Treatment | FW | REN | Error | N | | REN |
| TT8_{P′} - GGP.sub.UTR | 22 | 0.057 | 0.008 | 12 | a | 0.044 |
| TT8_{P′} - GGP.sub.UTR + | 66 | 0.007 | 0.001 | 12 | b | 0.0063 |
| GGP | | | | | | |
| TT8_{P′} - GGP.sub.UTR + | 24 | 0.041 | 0.002 | 12 | a | 0.041 |
| FG429343 | | | | | | |
| GGP_{P′} - TT8.sub.UTR | 18 | 0.861 | 0.112 | 12 | a | 0.753 |
| GGP_{P′} - TT8.sub.UTR + | 72 | 0.479 | 0.027 | 12 | b | 0.441 |
| GGP | | | | | | |
| GGP_{P′}′ - TT8.sub.UTR + | 22 | 0.553 | 0.085 | 12 | b | 0.492 |
| FG429343 | | | | | | |

The ascorbate down regulation of the GGP promoter is expressed through the 5′-UTR region of the gene. Subscript UTR refers to the 5′-UTR of the gene and subscript P′ refers to just the untranscribed promoter of the respective gene. GGP refers to the cotransformation of the CDS of GGP under the control of the 35S promoter in order to raise ascorbate levels while FG429343, is a methyl transferase control gene. The slope of LUC REN is the slope of the plot of LUC values against REN values forced through the origin. N is the number of independent LUC/REN ratios measured. Within a block of three rows, LUC/REN means with the same letter do not significantly differ at p < 0.01. Other details are found in the methods section

To investigate whether the ascorbate control is at the transcriptional or post-transcriptional level, Applicants measured transcript levels of the reporter gene construct. Our data show little effect of ascorbate on the levels of LUC mRNA (Table 6), indicating that ascorbate, directly or indirectly, acts through the 5′-UTR to control the translation of GGP.

TABLE 6
Effect of ascorbate level on the RNA levels for LUC driven either by the GGP
promoter or TT8, a control gene. Gene expression was measured relative to
the expression of REN in the same RNA preparation. Values are the mean of
three biological replicates, each involving three combined leaves. Standard
errors are shown brackets. Within each promoter pair, there was no significant
difference in gene expression or the LUC activity for the TT8 promoter. The
change in LUC activity for the GGP promoter was significant (p < 0.001)
as were the changes in ascorbate for both promoters (p < 0.003).

| | Gene | | Ascorbate |
|---|---|---|---|
| Promoter/Treatment | expression | LUC/REN | mg/100 g FW |
| GGP/low ascorbate | 6.0E-02 +−. 8.8E−03 | 1.08 .+−. 0.05 | 25.3 .+−. 0.93 |
| GGP/high ascorbate | 5.3E-02 +−. 7.4E−03 | 0.15 .+−. 0.03 | 92.4 .+−. 7.1 |
| TT8/low ascorbate | 8.5E-01 +−. 5.7E−02 | 19.9 .+−. 0.59 | 26.1 .+−. 6.3 |
| TT8/high ascorbate | 9.3E-01 +−. 1.1E−01 | 16.3 .+−. 0.83 | 84.6 .+−. 4.7 |

To verify that the 5′-UTR acts directly to affect leaf ascorbate concentrations, we constructed a 35S driven GGP coding sequence with and without the GGP 5′-UTR in front of the coding sequence. Both constructs enhanced leaf ascorbate in the transient system, but the construct without the 5′-UTR had about 30% more ascorbate than the construct with the 5′-UTR. Furthermore, co-infiltrating GME into the leaf so to drive the ascorbate even higher than GGP alone (Bulley et al., 2009, J. Exp. Bot. 60, 765.) resulted in over two-fold higher ascorbate in the construct without the 5′-UTR than the construct containing the 5′-UTR. Thus, in high ascorbate conditions the GGP 5′-UTR limits both GGP production and ascorbate synthesis. Removal of this regulation provides a way of generate plants with high ascorbate levels.

Given that the effect of ascorbate was mediated through the 5′-UTR region of GGP, we examined the properties of the 5′-UTR. GGP is unusual in having a long 5′-UTR, over 500 bp long in many species with strongly conserved elements. Aligned GGP 5′-UTRs from different species including an alga and two mosses revealed the presence of a highly conserved uORF with the potential to encode a 60 to 65 amino acid peptide. Interestingly, for this peptide to be made, translation would need to initiate at a non-canonical ACG initiation codon. A few examples of non-canonical translation initiation have been described (Ivanov et al., 2008, Proc. Natl. Acad. Sci. USA 105, 10079). Efficient translation requires Kozak sequences which is the case for this ORF. To test if this uORF is required for ascorbate dependent regulation of the GGP gene. Applicants mutated the potential ACG initiation codon to TCG. LUC activity from the mutated construct remained high, even in the presence of high ascorbate. To further examine the requirement of the uORF, Applicants mutated a highly conserved His (CGG codon) at residue 36 to Leu (CTG). Again, this abolished ascorbate dependent regulation. Mutating an internal ATG codon with potential to encode a short uORF of 10 amino acids, increased the relative LUC activity by over two-fold, probably because it removed a competing start codon, but did not change the relative sensitivity of the promoter to ascorbate concentration.

Applicants then used the ACG uORF mutant that did not respond to ascorbate to test whether the predicted uORF worked in a Cis or Trans configuration. Applicants tested whether expressing the ACG uORF separately could recover ascorbate repression of LUC activity in this mutated vector. The presence of the ACG uORF had no effect on any treatment and did not complement the mutant form of the ACG uORF. This is consistent with the uORF working in a Cis conformation with the GGP CDS.

In this specification Applicants provide evidence that ascorbate, or a precursor of ascorbate, interacts either directly or indirectly through an intermediate with a peptide produced by a non-canonical uORF in the 5′-UTR of GGP, the key control gene of ascorbate biosynthesis, resulting in inhibition of the translation of the GGP enzyme. Reports of the control of protein expression in eukaryotes by products of a biosynthesis pathway are rare. Often gene expression is controlled by signaling cascades via a separate receptor to a transcription factor or through posttranslational modification of the target proteins (Smeckens et al., 2010, Curr. Opin. Plant Biol. 13, 273). While it has been reported that 5′-UTR sequences are important in controlling protein expression (Hulzink et al., 2003, Plant Physiol. 132, 75), reports on the control by a small molecule of gene expression through the 5′-UTR of the mRNA in eukaryotes are uncommon (Rahmani et al., 2009, Plant Physiol. 150, 1356) and control through a non-canonical start codon uORF are extremely rare.

A simple model of action may be that the ACG uORF is translated but in the presence of high ascorbate, the ribosome is stalled on the uORF. At low ascorbate, the translation terminates at the stop codon and immediately restarts downstream at the start ATG of GGP. There is no obvious Kozak sequence associated with the GGP primary start codon, but a reasonably strong Kozak sequence associated with ACG1. This effectively primes the ribosome at high ascorbate on the GGP mRNA ready for translation to respond rapidly to any reduction in ascorbate due to stress.

It appears that another factor may be required for the action of this feed-back loop. This is because the ascorbate regulation of the 5′-UTR for GGP from A. eriantha, a kiwifruit species with very high fruit ascorbate (Bulley et al., 2009, J. Exp. Bot. 60, 765.), functions in N. benthamiana. For A. eriantha to have high ascorbate suggests a mutation has occurred disrupting the ascorbate feedback of GGP translation in A. criantha. However, that control of the A. criantha GGP by ascorbate is functional in N. benthamiana suggests a factor mediating between ascorbate and the ACGI uORF Is functional in N. benthamiana. This factor is likely to be a protein.

There are two types of uORF (Tran et al., 2008, BMC Genomics 9, 361): sequence-independent uORFs, where translation of the uORF influences the reinitiation efficiency of a downstream ORF and thus affects overall translation (Calvo et al., 2009. Proc. Natl. Acad. Sci. USA 106, 7507) but the uORF-encoded peptide sequence is not important (the short ATG 10 amino acid uORF In the GGP 5′-UTR appears to fit into the class, and sequence-dependent ORFs, where the nascent uORF peptide causes ribosome stalling during translational elongation and termination. The fact that the GGP uORF encodes a highly conserved peptide over a wide range of plant taxonomies and that ascorbate repression is abolished by a single amino acid mutation in the uORF, indicates the latter type. Two examples in plants, polyamine and sucrose regulation (Rahmani et al., 2009, Plant Physiol. 150, 1356; Gong and Pua, 2005, Plant Physiol. 138, 276) involve sequence dependent uORFs. Our new example is different in that it initiates translation with a highly conserved non-canonical codon.

In conclusion we have shown that the level of ascorbate in a leaf can be controlled through ascorbate feedback through a noncanonical uORF in the long 5′-UTR of the controlling gene of ascorbate biosynthesis. GGP. We show evidence that this feedback acts post-transcriptionally by controlling the level of the GGP enzyme. We propose that this is a major mechanism that ascorbate concentrations are controlled in the L-galactose pathway of ascorbate biosynthesis.

### Materials and Methods

### Plant Materials and Chemical Assays

The Nicotiana benthamiana leaf transient reporter gene system using luciferase (LUC) as the promoter specific reporter and renilla (REN) as the transformation reporter was as described previously (Hellens et al., 2005, Plant Methods 1, 13). Ascorbate concentration in the leaf was manipulated by co-injecting either the coding sequence for Actinidia chinensis GGP in pGreen (Hellens et al., 2000, Plant Mol. Biol. 42, 819) under the 35S promoter transformed into Agrobacterium tumifaciens (Bulley et al., 2009, J. Exp. Bot. 60, 765.) or a KO vector constructed using as a template the GGP sequence from N. benthamiana assembled from seven ESTs in GenBank as described by (Snowden et al., The Plant Cell 17, 746). In addition, the version of the CDS of A. eriantha GME (GenBank accession FG424114) described earlier (Bulley et al., 2009, J. Exp. Bot. 60, 765.) was used to synergistically enhance ascorbate with GGP. Ascorbate was measured in extracts of the same leaf using an HPLC based assay also as previously described (Rassam et al., 2005, J. Agric. Food Chem. 53, 2322). Ascorbate was measured as total ascorbate by reducing extracts before HPLC (Rassam et al., 2005, J. Agric. Food Chem. 53, 2322). Measurement of the redox state of the ascorbate found the redox potential decreased significantly with increased concentration of ascorbate. This raises the possibility that the effect of ascorbate may be either through ascorbate itself or through the decreased redox potential at increased ascorbate.

In different leaves of N. benthamiana, ascorbate affects the LUC activity but has little effect on the REN activity.

To test whether the effect of ascorbate was mediated by the promoter or the 5′-UTR we constructed two vectors where the 5′-UTR regions were swapped between the GGP promoter and the TT8 promoter. The resulting constructs consisted of the TT8 core promoter (TT8P′) followed by the GGP 5′-UTR (GGPUTR) and vice versa. We transiently expressed these in leaves and measured the relative LUC activity.

To test the effect of the 5′-UTR in front of the GGP coding sequence on ascorbate concentration, a different GGP gene (GenBank accession FG460629) was used instead of the GGP used in other experiments. At the protein level it was 96% identical to the standard GGP and in the absence of the 5′-UTR, raised ascorbate concentrations to similar levels seen for the usual GGP. The version with the 5′-UTR had the full 5′-UTR, while the version labeled without the UTR had all but 37 bp upstream of the start ATG deleted using the Xhol restriction enzyme. In this region at the 3′ end of the 5′-UTR, there is little homology between GGPs. Both versions were ligated into the pART277 vector (Gleave, A., 1992, Plant Mol. Biol. 20, 1203 (1992).

The amount of LUC protein was measured using an antibody to LUC (Promega) using a Western blot of 50 μg soluble cellular protein per lane (extracted in 40 mM phosphate buffer, pH 7.4. 150 mM NaCl) from various constructs transiently expressed in Nicotiana benthamiana leaves. The large subunit of RuBisCO as stained by SYPRO Tangerine protein gel stain is shown as a loading control.

To separate the effects of GGP protein from ascorbate, we initially attempted to use ascorbate or its precursors injected directly into the leaf by syringe. However, we could not get sustained changes in leaf ascorbate. We also tried allowing detached leaves or discs previously injected with Agrobacterium LUC/REN constructs to take up ascorbate precursors. While these leaves did have very significant increases in ascorbate, the leaves deteriorated before LUC/REN values could rise enough to be measurable. We then tried lowering ascorbate without lowering GGP concentrations by knocking out two genes involved in ascorbate biosynthesis (encoding galactose dehydrogenase and GDP mannose epimerase). However, again we failed to have significant changes in leaf ascorbate, suggesting that their expressed enzymes may be stable or in excess over the seven-day extent of the experiment.

Experiments were repeated at least twice with similar results, and although in some cases the high ascorbate reduced REN expression as well as LUC, this did not alter the effect of ascorbate on reducing the slope of the relationship (i.e., the LUC/REN ratio) for the GGP promoter but not the TT8.

### Gene Cloning and Plasmids

The GGP promoter from Actinidia eriantha (SEQ ID NO: 101) was cloned by genome walking and has been deposited in GenBank as accession number JX486682. A. criantha gDNA (2.0 μg) was digested using seven blunt cutting restriction enzymes; DraI, EcIII 136, EcoRV, HpaI, MScI, ScaI, SspI and StuI. Digests were purified and eluted in 10 μL using PCR Clean and Concentrate spin columns (Zymogen). Double stranded adapter sequences (Clontech) containing nested PCR primer sites were ligated onto cut fragments overnight at 16° C., using T4 Rapid Ligase (Roche). Ligations were column purified a second time and eluted in 30 μL. First round PCR was performed using 1 μL of each digest with primers 319998NRWLK1. RPH-149 and Ex Taq polymerase (Takara) using the following two step cycling conditions. For the first high stringency step, one cycle of denaturation was performed at 94° C., for two min, followed by seven cycles of 94° C., for 25 sec and elongation/annealing at 72° C., for three min. The second step consisted of 32 cycles of 94° C., for 25 sec and 67° C., for three min before a final 67° C. extension for three min. The first-round products were run on a 1% agarose gel and 1 μL of a 1:50 dilution was used as template for second round PCR with 319998NRWLK2 and RPH-150. Second round PCR was also performed as a two-step PCR with an initial denaturation of 94° C, for two min followed by five cycles of 94° C. for 25 sec and 72° C. for 3 m. This was followed by a further 20 cycles of 94° C. for 25 s and 67° C. for three min before a final 67° C. extension. Gel electrophoresis was used to identify PCR products between 500-2 kb in size for cloning into pGem T Easy vector (Promega) according to the manufacturer's instructions. Clones were DNA sequenced confirmed for overlap with the known 5′-UTR. A second set of nested primers was designed to the end of the first promoter walk to extend the known A. eriantha promoter sequence to 2 kb. This 2 kb promoter sequence was then PCR amplified from A. eriantha gDNA using primers Eriantha gDNA PCR 5′ and 319998NRWLK2 and sub-cloned into pGreen0800-5′_LUC (Hellens et al., 2000, Plant Mol. Biol. 42, 819) using EcoRV and Ncol restriction enzymes. The final construct is called the GGP-promoter-pGreenII 0800-5 LUC vector.

The promoter for GGP from Arabidopsis thaliana (At4g26850) (SEQ ID NO: 102) and control promoters from a range of sources were cloned by PCR. The control promoters were TT8 (AT+G09820). EF1.alpha. (AT1G07940). Act2P (AT3G18780) and Act7P (AT5G09810).

Generation of the inactivated start codons or other deletions and mutations in the uORFs of the 5′-UTR was done by chemical synthesis (GenScript, www.genscript.com) of mutated and control sequences. In the inactivated versions, the ATG or ACG start codons of the uORFs were changed to a TTG. Other changes were done by site-specific mutagenesis. There is a Stul site on the 5′ side of the UTR 28 bp into the 5′-UTR which was used as the 5′ border of the synthesized fragment. We added an extra CC to the 3′ end of the synthesized genes to create a Ncol site (ccatgg) and removed the sequence equivalent to the synthesized fragment from the GGP-promoter-pGreenII 0800-5 LUC by digesting it with Stul and Ncol. Then the synthesized fragments were separately cloned into the vector to create the two versions with and without a uORF.

RNA Isolation and cDNA Synthesis

Total RNA was isolated from 100 mg leaf tissue using a RNeasy Plant Mini kit (Qiagen) and concentrations were measured using a Nanodrop 1000 spectrophotometer (Thermo Fisher Scientific Inc.). Complementary DNA was then synthesized from 1 μg total RNA and random hexamers in a 10 μl total volume using a BluePrint Reagent Kit for Real Time PCR (Takara Bio Company) following manufacturer instructions. Following cDNA synthesis, the preparation was diluted 75 times in preparation for quantitative real time PCR.

### Quantitative PCR

Quantitative PCR was performed in 5 μl total volume using a LightCycler.RTM. 480 Real-Time PCR System (Roche Diagnostics) and the following primer pair: LUC1/2: 5′-TATCCGCTGGAAGATGGAAC-3′ (SEQ ID NO: 109): 5′-TCCACCTCGATATGTGCATC-3′. (SEQ ID NO: 110) Primers were designed with annealing temperatures of 60° C. using Primer3 (Rozen and Skaletsky, 2000, Methods Mol Biol. 132, 365. The luciferase primer pair amplifies regions from the 5′-end of the luciferase open reading frame. Reaction components (using LightCycler 480.RTM. SYBR Green I Master Mix) were as follows: 2 μM each primer, 1.25 μl diluted cDNA preparation. The standard cycling protocol with a Tm of 60° C. was used and relative quantification analysis normalized to Renilla transcripts was performed using The LightCycler.RTM. 480 Software (Roche Diagnostics).

### Control Tests of the System Used

As the control gene promoter TT8 expressed about 10 times higher than the GGP promoter we considered the possibility that the TT8 promoter might have saturated the ability of the tobacco cell to express the transcript or translate the LUC and thus any inhibition would not be seen. To check this, we titrated the amount of Agrobacterium containing the TT8-LUC construct over a 200-fold range. The ratio of LUC/REN and the slope of the relationship between LUC and REN was unchanged by the amount of Agrobacterium injected (Table 2) showing little sign of any saturation of expression of the reporter genes. The TT8 promoter driven LUC values overlapped with the LUC values expressed using the GGP promoter. We also tested whether several different alternative promoters were inhibited by ascorbate. These included EF1.alpha., Act2P, and Act7P. None of these promoters were negatively affected by ascorbate (Table 3) although in this experiment, the TT8 promoter strength was actual increased by ascorbate. In a third test we verified that the effect of ascorbate on the GGP promoter strength was not restricted to the kiwifruit GGP gene promoter by testing the same promoter from Arabidopsis GGP (At4g26850). The response of the LUC/REN ratio to ascorbate was essentially identical for the two promoters from different species. In a final test, we checked whether expressing a gene to raise ascorbate (kiwifruit GGP) might in itself affect the results. Consequently, we added an extra control gene in the form of a methyl transferase. While we got a small reduction in this experiment in the LUC/REN ratio of the added control gene (Table 4), this did not change the conclusion that increased ascorbate reduced the strength of the GGP promoter but had little effect of other control promoters (Liang et al. U.S. Pat. No. 9,648,813).

### Example 2. Testing the Effect of Ascorbate on Other 5′UTR Sequences in the LUC/REN Reporter Assay

### Methods

A 35S driven-LUC construct was derived from pGreen 0800 LUC (Hellens et al., 2000, Plant Mol. Biol. 42, 819), where a second copy of the 35S promoter without its 5′UTR was cloned into the multiple cloning site in front of the LUC coding sequence, and 5′UTRs from apple, potato, and tomato (SEQ ID NO: 126, 127 and 128 respectively) were inserted between this 35S promoter and the beginning of the LUC coding sequence replacing the 35S 5′UTR. The 5′UTR sequences appear in CAPITAL letters within SEQ ID Nos: 126-128.

Tomato GGP 5′ UTR
(SEQ ID NO: 126)
atttgttcggtatactgtaaccccctgtttgcgattggccttgtagccccgttttacatcttccagagactccatttgtatcggttcacata
cagtagcaaagcgccattatcttactctaccccattggcaaacccacagccacaattttccaatcctccattatcccttctacaattttcta
tataaatacccacatctctctgctctactcccttattatcaacaacaaccaccaaatttcttcttttttttcttcgatagtagcaatctatc
aacaaaaacagagaccccatcacaagaatcttggaattttagtgttgggtttaagaggaaaaggggttattgtattttgcagttttgagggt
aaagcccagtttaacaagttgtagacatACGGCTATACACAAAGTAAACCGCCGACCACTTTTACATGTTCCAGCAGTACGTCGTAAGGGTT
GTGTAACAGCTACTAACCCTGCGCCGCACGGTGGACGTGGCGCTTTGCCTTCTGAAGGTGGTAGTC
CTTCCGACCTCCTCTTCCTTGCCGGCGGCGGTTCTTTCCTCTCCTTCTCCTACtagatatagtta
tacttactatagatctctagcttattacgtacagttgtatctagtattctattgattattcgaagaaaacacacaaaaagaagtaaagcc
Potato GGP 5′ UTR
(SEQ ID NO: 127)
taagggggtgcttatataaagttggggagtctaccaatgagacgaactcattgaccaaatacgtctgcaggagaaagaccaccggagcacca
aacgccacccaacaaccacccattaaattcttccagaaaaaaacatcttcctcaaaattatcgatgaaggatcgttccttagtagttg180t
tcgttgatcctacaaattcaatcACGGCTCTTCTTGGATCTTTCGTTTGTATTCTCACAATTCATCATCACCGCAAAGT
GTTGACCCTTAATCCAACTCTTCTGGTGGACGATAAGCACCGGACCCCTTCCCCTCAC
GGAGGTAGGGGTGCCTCACCCGCTGAAGGCGGTTGCCCCTCCGATCTCCTCTTCCTCG
CCGGCGGCGGTCCAATTCTTCCTTTCTCTTTCTCCTTCTCCtaatttttcgtgtaagaattgtatttttgattatcc
atccaagaacaggaccgcc
Apple GGP 5′ UTR
(SEQ ID NO: 128)
ccacggtacaccctcagccacgaacaccccttcttctccccacacctataaatccaccccctcatctcctccccacacccccactcacttca
gttegaaacaggcgatcctcgcctttctgggttgtttcctattttatctgagggagaagaaaggaaggtgtttgatcaattttttggtatat
ttttaggggtaagacccaggttcgacgagttgtagacatcACGGCTATACACGGAGCTCCTCGGCCGCTCATTCATGTCCGGGCTGTC
CGACGAAAGGGTTGTGTAATTGAGAGCAACCCTTCGCCGCACGGCGGGCGTGGCGCT
TTGCCTTCCGAAGGCGGTAGCCCCTCCGACCTGCTCTTCCTCGCTGGTGGCGGTTCTG
CATCCTCTGTTTTTCTCTTCTGCTTATATtagcttttttagactttcttggttagattcttaggagattttagagattttttttcttcta
taaagcgcacgagtagatcgtattgttgttttcggggggttttgggtttggtggtgtttgattttactgagaattaagaaaaaataaaagga
aaaaaaagagagagagaaagaaggggagggagcatgcc

These constructs were tested as described for other GGP promoter constructs in Example 1 above.

### Results

The LUC genes, each driven by different GGP 5′UTR inserts, were all down regulated by ascorbate (FIGS. 16, 17 and 18, Table 7). In each case both the LUC/REN ratios and the slope of the relationship between LUC and REN were reduced by ascorbate. In these experiments, the ascorbate increased less than shown in Example 1 (Table 7), possibly due to varying growth conditions (lower light and higher temperature) but the increased ascorbate still reduced the LUC values significantly. Also shown are corresponding data for a typical non-responsive promoter-5′UTR (EF1alpha) and for the standard GGP promoter-5′UTR. The further variant 5′UTR sequences disclosed can of course be tested in the same way (Liang et al. U.S. Pat. No. 9,648,813).

TABLE 7

| | | | | | | Mean | |
|---|---|---|---|---|---|---|---|
| | Mean | | | | | ascorbate | |
| | LUC/ | Std | | | | mg/100 g | Std |
| 5′UTR | REN | error | p | Slope | R² | FW | error |
| Tomato low ascorbate | 1.88 | 0.21 | 0.000 | 0.77 | 0.30 | 19.1 | 1.7 |
| Tomato high ascorbate | 0.69 | 0.22 | | 0.04 | 0.00 | 36.6 | 1.5 |
| Potato low ascorbate | 2.03 | 0.22 | 0.000 | 1.55 | 0.57 | 21.5 | 1.4 |
| Potato high ascorbate | 0.30 | 0.04 | | 0.13 | 0.63 | 40.3 | 3.4 |
| Apple low ascorbate | 1.77 | 0.24 | 0.024 | 2.31 | 0.78 | 18.4 | 1.1 |
| Apple high ascorbate | 0.89 | 0.26 | | 0.41 | 0.34 | 33.6 | 2.9 |
| EF1alpha promoter | 1.821 | 0.15 | 0.028 | 1.51 | 0.71 | 19.1 | 0.6 |
| low ascorbate | | | | | | | |
| EF1alpha promoter | 2.278 | 0.13 | | 2.21 | 0.85 | 34.9 | 2.0 |
| high ascorbate | | | | | | | |
| Kiwifruit GGP-5′UTR | 0.236 | 0.02 | 0.000 | 0.27 | 0.77 | 20.5 | 0.8 |
| low ascorbate | | | | | | | |
| Kiwifruit GGP-5′UTR. | 0.079 | 0.01 | | −0.03 | 0.08 | 40.3 | 1.9 |
| high ascorbate | | | | | | | |

Tabulation of LUC/REN ratios and slopes for plots of LUC against REN for various 5′UTR from GGPs from three different species. We also include a control promoter and its own 5′UTR and the original GGP promoter and 5′UTR used previously as controls. The p value is the statistical significance for the difference between high and low ascorbate for each construct.

### Example 3. Identifying uORFs in Orthologous Proteins by BLAST Analysis Using the Main ORF Encoded Polypeptide

Sequence similarity between mORFs of different species can be used to identify uORFs in heterologous genes that encode polypeptides that are homologs. As an example, AT1G01060.1 (LHY) encodes a MYB-related putative transcription factor involved in circadian rhythm and was identified as a new uORF-containing gene candidate. The protein sequence of LHY from Arabidopsis was used to identify the LHY orthologs in Brassica oleraceae. The ATIG01060.1 sequence was then used in a sequence homology alignment search of the genome Brassica oleracea using BLAST (tblastn) at genomevolution.org/coge/CoGeBlast.pl as well as in a range of other species Results are shown in Table 8. Similarly, in this application, we have used mORFs encoding GGP from one species to identify GGP proteins from other species.

TABLE 8
Putative orthologs of LHY/CCA1 from sugar beet (Beta vulgaris) and Eucalyptus (Eucalyptus grandis),
barrel medic (Medicago truncatula) and brassica sp. (Brassica oleracea) through BLAST analysis

| | | | | | | Closest |
|---|---|---|---|---|---|---|
| | | | | | | Genomic |
| | Chr | Position | HSP# | E−value | Quality | Feature |
| Beta vulgaris (Andrew Funk | 1_EL10.1 | 2029057 | 1 | 2.00E−38 | 21.80% | E10Ac1g00171.1: 8 |
| FASTA vEL10 1.0) | | | | | | |
| Beta vulgaris (Andrew Funk | 4_EL10.1 | 36341401 | 2 | 2.00E−19 | 7.40% | none |
| FASTA vEL10_1.0) | | | | | | |
| Beta vulgaris (Andrew Funk | 1_EL10.1 | 2027891 | 3 | 2.00E−38 | 9.30% | E10Ac1g00170.1: 13 |
| FASTA VEL10_1.0) | | | | | | |
| Beta vulgaris (Andrew Funk | 1_EL10.1 | 2040547 | 4 | 7.00E−15 | 5.30% | E10Ac1g00171.1: 4 |
| FASTA vEL10_1.0) | | | | | | |
| Beta vulgaris (Andrew Funk | 7_EL10.1 | 29147606 | 5 | 1.00E−06 | 4.00% | none |
| FASTA vEL10_1.0) | | | | | | |
| Eucalyptus grandis BRASUZ1 | 4 | 40431588 | 1 | 7.00E−47 | 0.329 | Eucgr.D02651.2.v2.0 |
| Eucalyptus grandis BRASUZ1 | 6 | 41227391 | 2 | 5.00E−19 | 0.048 | Eucgr.F02906.1.v2.0 |
| Eucalyptus grandis BRASUZ1 | 4 | 40426987 | 3 | 1.00E−12 | 0.053 | Eucgr.D02651.1.v2.0 |
| Eucalyptus grandis BRASUZ1 | 11 | 30919289 | 4 | 7.00E−19 | 0.043 | Eucgr.K02302.1.v2.0 |
| Eucalyptus grandis BRASUZ1 | 1 | 42597650 | 5 | 0.000004 | 0.037 | Eucgr.A02733.1.v2.0 |
| Eucalyptus grandis BRASUZ1 | 11 | 30919483 | 6 | 7.00E−19 | 0.031 | Eucgr.K02302.1.v2.0 |
| Eucalyptus grandis BRASUZ1 | 6 | 41227587 | 7 | 5.00E−19 | 0.029 | Eucgr.F02906.1.v2.0 |
| Eucalyptus grandis BRASUZ1 | 4 | 40426881 | 8 | 1.00E−12 | 0.012 | Eucgr.D02651.1.v2.0 |
| Medicago truncatula (barrel | 7 | 49122314 | 1 | 2.00E−42 | 32.60% | Medtr7g118330.1.JCVIMt4.0v1 |
| medic) | | | | | | |
| Medicago truncatula (barrel | 6 | 28640709 | 2 | 3.00E−18 | 7.00% | Medtr6g477860.2.JCVIMt4.0v1 |
| medic) | | | | | | |
| Medicago truncatula (barrel | 7 | 49115772 | 3 | 2.00E−21 | 5.40% | Medtr7g118330.1.JCVIMt4.0v1 |
| medic) | | | | | | |
| Medicago truncatula (barrel | 5 | 32830655 | 4 | 1.00E−12 | 4.50% | Medtr5g076960.2.JCVIMt4.0v1 |
| medic) | | | | | | |
| Medicago truncatula (barrel | 3 | 29065130 | 5 | 1.00E−18 | 4.80% | Medtr3g064500.1.JCVIMt4.0v1 |
| medic) | | | | | | |
| Medicago truncatula (barrel | 7 | 49115962 | 6 | 2.00E−21 | 3.10% | Medtr7g118330.1.JCVIMt4.0v1 |
| medic) | | | | | | |
| Medicago truncatula (barrel | 3 | 29065360 | 7 | 1.00E−18 | 2.80% | Medtr3g064500.1.JCVIMt4.0v1 |
| medic) | | | | | | |
| Brassica oleracea (Ensembl | C5 | 409337 | 1 | 2.00E−126 | 40.30% | CDS: Bo5g002760.1 |
| Plants Masked by source v2.1) | | | | | | |
| Brassica oleracea (Ensembl | C5 | 410562 | 3 | 2.00E−126 | 14.70% | CDS: Bo5g002760.1 |
| Plants Masked by source v2.1) | | | | | | |
| Brassica oleracea (Ensembl | C3 | 37760 | 4 | 5.00E−71 | 26.00% | CDS: Bo3g001090.1 |
| Plants Masked by source v2.1) | | | | | | |
| Brassica oleracea (Ensembl | C3 | 38526 | 6 | 5.00E−71 | 13.30% | CDS: Bo3g001090.1 |
| Plants Masked by source v2.1) | | | | | | |
| Brassica oleracea (Ensembl | C3 | 4200012 | 8 | 2.00E−21 | 6.70% | CDS: Bo3g012470.1 |
| Plants Masked by source v2.1) | | | | | | |

### Example 4. Improvement of Traits in Rice Through Elevated Expression of a GGP Protein

A rice plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding a rice GGP protein or its homolog is fused to a heterologous promoter (such as, but not limited to, rice actin, tubulin. 35S, a drought inducible promoter, RD29A, RD29B, or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into rice cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 5. Improvement of Traits in Rice Through Elevated Expression of a GGP Protein, Obtained by Generation of a Non-Naturally Occurring Allele Comprising a Mutation in the uORF of a GGP Gene

A rice plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding a rice GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the rice genome that produces a lower level of luciferase activity than the deletion mutation but a higher level than the construct carrying an unmutated 5′UTR of the GGP gene. The selected mutation is then engineered by means of a guide RNA which is introduced into rice cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 6. Improvement of Traits in Rice Through Elevated Expression of a GGP Protein

A wheat plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding a wheat GGP protein or its homolog is fused to a heterologous promoter (such as, but not limited to, rice actin, tubulin. 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into wheat cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 7. Improvement of Traits in Wheat Through Elevated Expression of a GGP Protein, Obtained by Generation of a Non-Naturally Occurring Allele Comprising a Mutation in the uORF of a GGP Gene

A wheat plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding a wheat GGP protein (Seq ID), which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the wheat genome that produces a lower level of luciferase activity than the deletion mutation but a higher level than the construct carrying an unmutated 5′UTR of the GGP gene. The selected mutation is then engineered by means of a guide RNA which is introduced into wheat cells to produce the selected gene edit, or an allele of comparable strength. Cells carrying the targeted mutation engineered into their genomes arc then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 8. Improvement of Traits in Corn Through Elevated Expression of a GGP Protein

A corn plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding a corn GGP protein (SEQ ID NO: 19) or its homolog is fused to a heterologous promoter (such as, but not limited to, rice actin, tubulin, 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into corn cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 9. Improvement of Traits in Corn Through Elevated Expression of a GGP Protein, Obtained by Generation of a Non-Naturally Occurring Allele Comprising a Mutation in the uORF of a GGP Gene

A corn plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding a corn GGP protein (SEQ ID NO: 99), which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the corn genome that produces a lower level of luciferase activity than the deletion mutation but a higher level than the construct carrying an unmutated 5′UTR of the GGP gene. The selected mutation is then engineered by means of a guide RNA which is introduced into corn cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 10. Improvement of Traits in Soybean Through Elevated Expression of a GGP Protein

A soy bean plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding a soybean GGP protein (SEQ ID NO: 3) or its homolog is fused to a heterologous promoter (such as, but not limited to, rice actin, tubulin, 35S, a drought inducible promoter, RD29A, RD29B, or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into soy bean cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 11. Improvement of Traits in Soybean Through Elevated Expression of a GGP Protein, Obtained by Generation of a Non-Naturally Occurring Allele Comprising a Mutation in the uORF of a GGP Gene

A soy bean plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding a soy bean GGP protein (SEQ ID NO: 83), which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the soy bean genome that produces a lower level of luciferase activity than the deletion mutation but a higher level than the construct carrying an unmutated 5′UTR of the GGP gene. The selected mutation is then engineered by means of a guide RNA which is introduced into soybean cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 12. Improvement of Traits in Alfalfa Through Elevated Expression of a GGP Protein

An alfalfa plant that exhibits the phenotype of biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding an alfalfa GGP protein or its homolog is fused to a heterologous promoter (such as, but not limited to, rice actin, tubulin, 35S, a drought inducible promoter, RD29A, RD29B, or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into alfalfa cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 13. Improvement of Traits in Alfalfa Through Elevated Expression of a GGP Protein, Obtained by Generation of a Non-Naturally Occurring Allele Comprising a Mutation in the uORF of a GGP Gene

An alfalfa plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding an alfalfa GGP protein which have sequence changes in a uORF are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the alfalfa genome that produces a lower level of luciferase activity than the deletion mutation but a higher level than the construct carrying an unmutated 5′UTR of the GGP gene. The selected mutation is then engineered by means of a guide RNA which is introduced into alfalfa cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 14. Improvement of Traits in Sugarcane Through Elevated Expression of a GGP Protein

A sugarcane plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene (for example. SEQ ID NO: 153) encoding a sugarcane GGP protein or its homolog is fused to a heterologous promoter (such as, but not limited to, rice actin, tubulin, 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into sugarcane cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 15. Improvement of Traits in Sugarcane Through Elevated Expression of a GGP Protein, Obtained by Generation of a Non-Naturally Occurring Allele Comprising a Mutation in the uORF of a GGP Gene

A sugarcane plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene (for example. SEQ ID NO: 153) encoding a sugarcane GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the sugarcane genome that produces a lower level of luciferase activity than the deletion mutation but a higher level than the construct carrying an unmutated 5′UTR of the GGP gene. The selected mutation is then engineered by means of a guide RNA which is introduced into sugarcane cells to produce the selected gene edit, or an allele of comparable strength. Cells carrying the targeted mutation engineered into their genomes are then selected and regenerated into plant lines. Each regenerated plant line, or a descendant of it (produced through selfing. crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 16. Improvement of Traits in Miscanthus Through Elevated Expression of a GGP Protein

A Miscanthus plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding a Miscanthus GGP protein (SEQ ID NO: 151) or its homolog is fused to a heterologous promoter (such as, but not limited to, rice actin, tubulin, 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into Miscanthus cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 17. Improvement of Traits in Miscanthus Through Elevated Expression of a GGP Protein, Obtained by Generation of a Non-Naturally Occurring Allele Comprising a Mutation in the uORF of a GGP Gene

A Miscanthus plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding a Miscanthus GGP protein (SEQ ID NO: 150), which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the Miscanthus genome that produces a lower level of luciferase activity than the deletion mutation but a higher level than the construct carrying an unmutated 5′UTR of the GGP gene. The selected mutation is then engineered by means of a guide RNA which is introduced into Miscanthus cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 18. Improvement of Traits in Almond Through Elevated Expression of a GGP Protein

An almond plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding an almond GGP protein or its homolog is fused to a heterologous promoter (such as, but not limited to, rice actin, tubulin, 35S, a drought inducible promoter, RD29A, RD29B, or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into almond cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 19. Improvement of Traits in Almond Through Elevated Expression of a GGP Protein, Obtained by Generation of a Non-Naturally Occurring Allele Comprising a Mutation in the uORF of GGP Gene

An almond plant that exhibits the phenotype of increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding an almond GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the almond genome that produces a lower level of luciferase activity than the deletion mutation but a higher level than the construct carrying an unmutated 5′UTR of the GGP gene. The selected mutation is then engineered by means of a guide RNA which is introduced into almond cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 20. Improvement of Traits in Eucalyptus Through Elevated Expression of a GGP Protein

A Eucalyptus plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. The gene encoding a Eucalyptus GGP protein or its homolog is fused to a heterologous promoter (such as, but not limited to, rice actin, tubulin, 35S, a drought inducible promoter, RD29A, RD29B, or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into almond cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 21. Improvement of Traits in Eucalyptus Through Elevated Expression of a GGP Protein, Obtained by Generation of a Non-Naturally Occurring Allele Comprising a Mutation in the uORF of a GGP Gene

A Eucalyptus plant that exhibits the phenotype of increased biomass, and/or increased shoot number and/or increased nutrient use efficiency and/or increased drought tolerance and/or increased tolerance to other abiotic stress and/or increased disease tolerance and/or tolerance to oxidative stress and/or tolerance to high cellular levels of free radicals is created as follows. Mutated variants of the 5′UTR from a gene encoding a Eucalyptus GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the almond genome that produces a lower level of luciferase activity than the deletion mutation but a higher level than the construct carrying an unmutated 5′UTR of the GGP gene. The selected mutation is then engineered by means of a guide RNA which is introduced into Eucalyptus cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 22. Improvement of Traits in Tomato Through Elevated Expression of a GGP Protein

A tomato plant that exhibits the phenotype of increased vitamin C content and/or increased biomass and/or increased shoot number and/or increased nutrient use efficiency and/or increased drought tolerance and/or increased disease tolerance and/or tolerance to oxidative stress and/or tolerance to high cellular levels of free radicals is created as follows. The gene encoding a tomato GGP protein or its homolog is fused to a heterologous promoter (such as, but not limited to, rice actin, tubulin, 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into tomato cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 23. Improvement of Traits in Tomato Through Elevated Expression of a GGP Protein, Obtained by Generation of a Non-Naturally Occurring Allele Comprising a Mutation in the uORF of a GGP Gene

A tomato plant that exhibits the phenotype of increased vitamin C content and/or increased biomass and/or increased shoot number and/or increased nutrient use efficiency and/or increased drought tolerance and/or increased disease tolerance and/or tolerance to oxidative stress and/or tolerance to high cellular levels of free radicals is created as follows. Mutated variants of the 5′UTR from a gene encoding a tomato GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the tomato genome that produces a lower level of luciferase activity than the deletion mutation but a higher level than the construct carrying an unmutated 5 ′UTR of the GGP gene. The selected mutation is then engineered by means of a guide RNA which is introduced into tomato cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 24. Improvement of Traits in Potato Through Elevated Expression of a GGP Protein

A potato plant that exhibits the phenotype of increased biomass and/or increased shoot number and/or increased nutrient use efficiency and/or increased drought tolerance and/or increased disease tolerance and/or tolerance to oxidative stress and/or tolerance to high cellular levels of free radicals is created as follows. The gene encoding a potato GGP protein or its homolog is fused to a heterologous promoter (such as, but not limited to, rice actin, tubulin, 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into wheat cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 25. Improvement of Traits in Potato Through Elevated Expression of a GGP Protein, Obtained by Generation of a Non-Naturally Occurring Allele Comprising a Mutation in the uORF of a GGP Gene

A potato plant that exhibits the phenotype of increased biomass and/or increased shoot number and/or increased nutrient use efficiency and/or increased drought tolerance and/or increased disease tolerance and/or tolerance to oxidative stress and/or tolerance to high cellular levels of free radicals is created as follows. Mutated variants of the 5′UTR from a gene encoding a potato GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the potato genome that produces a lower level of luciferase activity than the deletion mutation but a higher level than the construct carrying an unmutated 5′UTR of the GGP gene. The selected mutation is then engineered by means of a guide RNA which is introduced into potato cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 26. Improvement of Traits in Avocado Through Elevated Expression of a GGP Protein

An avocado plant that exhibits the phenotype of increased biomass and/or increased shoot number and/or increased nutrient use efficiency and/or increased drought tolerance and/or increased disease tolerance and/or tolerance to oxidative stress and/or tolerance to high cellular levels of free radicals is created as follows. The gene encoding an avocado GGP protein or its homolog is fused to a heterologous promoter (such as, but not limited to, rice actin, tubulin, 35S, a drought inducible promoter, RD29A, RD29B or the promoter from Arabidopsis gene AT5G43840, or a disease inducible promoter such as the promoter from Arabidopsis gene AT1G35230 or disease inducible promoters disclosed in U.S. Pat. No. 7,994,394) and transformed into avocado cells through use of an expression vector. Different transformant lines (aka events) harboring the transgene insertion are then regenerated and lines are tested in an oxidative stress assay, a disease stress assay, a heat stress assay, a salt stress assay, or a dry down assay whereby water is withheld from the plants until a population of control plants that lack the transgene show visible stress symptoms such as wilting. Individual lines are selected which show a lower level of stress symptoms than the control.

### Example 27. Improvement of Traits in Avocado Through Elevated Expression of a GGP Protein, Obtained by Generation of a Non-Naturally Occurring Allele Comprising a Mutation in the uORF of GGP Gene

An avocado plant that exhibits the phenotype of increased biomass and/or increased shoot number and/or increased nutrient use efficiency and/or increased drought tolerance and/or increased disease tolerance and/or tolerance to oxidative stress and/or tolerance to high cellular levels of free radicals is created as follows. Mutated variants of the 5′UTR from a gene encoding an avocado GGP protein, which have sequence changes in a uORF (or putative uORF), are each fused to a reporter gene encoding luciferase. A control construct is created in which the 5′ UTR from a GGP has its entire uORF deleted. The constructs are introduced into Nicotiana benthamiana cells or other eukaryotic cells and levels of expression of the reporter are compared. A mutation is selected for introduction into the avocado genome that produces a lower level of luciferase activity than the deletion mutation but a higher level than the construct carrying an unmutated 5′UTR of the GGP gene. The selected mutation is then engineered by means of a guide RNA which is introduced into avocado cells to produce the selected gene edit, or an allele of comparable strength. Cells carrying the targeted mutation engineered into their genomes are then selected and regenerated into plant lines. Each regenerated plant line, or a descendant of it (produced through selfing, crossing or cloning) is tested in a salt tolerance assay, or an oxidative stress assay, or a heat tolerance assay, or a dry down assay whereby water is withheld from the plants until a population of control plants that lack the gene edited mutation show visible stress symptoms such as wilting. Individual lines are selected which show a lower level of stress symptoms than the control.

### Example 28. Use of an Allele Comprising a Mutated uORF in a Tomato GGP Gene in the Heterozygous Condition to Obtained Desired Traits Without Negative Effects on Growth or Organ Development

A tomato plant with an elevated level of GGP protein is obtained through means of it containing a non-naturally occurring allele comprising a mutation in the uORF of a tomato GGP gene, whereby the tomato plant exhibits dwarfing and/or floral organ abnormalities (such as aberrant anthers, fused floral organs, and/or failure to produce pollen and/or reductions in pollen levels). The aforementioned plant is used as the female parent and pollinated to a cross to a wild-type tomato, or a heterozygote for the allele, to produce F1 seed. When grown the F1 seed develop into F1 plants that appear wild-type, or essentially wild-type, with few if any abnormalities, but with elevated levels of ascorbate in the fruit as compared to wild-type tomatoes.

### Example 29. A Method for Selecting a Target Mutation to Be Introduced Into the 5′UTR Upstream of a Gene of Interest That Produces a Desirable Trait in a Plant

The method includes introducing a mutation into uORF within the 5′UTR and fusing the mutated 5′UTR to a polynucleotide that encodes a reporter protein to create a polynucleotide fusion,
introducing the polynucleotide fusion into a test plant or plant cell, wherein the 5′UTR comprises at least one of:
(a) a sequence with at least 70% identity to any one of SEQ ID NOs: 41-100, 111-131, 144-145, 147, 149-150, and 153-155; or
(b) a sequence encoding a polypeptide with at least 70% identity to any one of SEQ ID NOs: 1-40, 108, 132-137, 146, 148, 151-152, and 156; wherein the mutation disrupts the function of an upstream open reading frame (uORF) encoded by the 5′UTR; and wherein said modification is at least one of a deletion, an addition, or a substitution of at least one nucleotide in the 5′UTR; and wherein the mutation results in the level of a product of the reporter gene in the test plant or plant cell being about 10%, about 20%, about 30%, about 40%, about 50%, about 60%, about 70%, about 80%, or about 90% of the level of reporter product produced in a control plant comprising a control 5′ UTR:reporter polynucleotide in which the uORF has been rendered inoperable by partial or complete deletion from the 5′ UTR fused to the reporter gene; and
selecting the mutation that resulted in said level of reporter gene product, or a comparable mutation, for introduction into the 5′UTR by gene editing at its endogenous locus in the plant genome.

### Example 30. A Selected Plant Exhibiting a Desirable Trait

In this example the selected plant comprises:
a nucleic acid comprising a 5′ UTR (untranslated region) upstream of a gene of interest that directly or indirectly controls the desirable trait;
wherein the 5′ UTR comprises a mutation in an upstream open reading frame (uORF), created by a targeted gene edit and wherein the sequence change to be produced through the edit was selected through a process whereby the mutated 5′UTR was fused to polynucleotide encoding a reporter protein and introduced into a test plant or plant cell and wherein the 5′UTR comprises at least one of:
   i) a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID NOs: 41-100, 111-131, 144-145, 147, 149-150, or 153-155; or
   ii) ii) a sequence encoding a polypeptide with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID NOs: 1-40, 108, 132-137, 146, 148, 151-152, or 156, wherein the mutation disrupts the function of an upstream open reading frame (uORF) encoded by the 5′UTR; and wherein said modification is at least one of a deletion, an addition, or a substitution of at least one nucleotide in the 5′UTR;
wherein the level of a product of the reporter gene in the test plant or plant cell is about 10%, about 20%, about 30%, about 40%, about 50%, about 60%, about 70%, about 80%, or about 90% of the level of a level of reporter product produced in a control plant comprising a control 5′ UTR::reporter polynucleotide in which the uORF has been rendered inoperable by partial or complete deletion from the 5′ UTR fused to the reporter gene;
wherein the plant has been selected for the desirable trait from a pool of plants that comprise the nucleic acid; and
wherein the desirable trait is selected from the group consisting of increased shoot number, decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and increased oxidative stress tolerance.

EXAMPLE 31. The selected plant of example 30, wherein the level of the product in the test plant or plant cell is about 10%, about 20%, about 30%, about 40%, about 50%, about 60%, about 70%, about 80%, or about 90%, or less than 100% of the level of the reporter product produced in the control plant cell

EXAMPLE 32. The selected plant of Example 30, wherein the desirable trait is selected from the group consisting of increased decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and increased oxidative stress tolerance.

### Example 33. A Method for Producing a Selected Plant Cell or Plant Selected for a Desirable Trait

In this method, the desirable trait is selected from the group consisting of: increased ascorbate level, increased biomass, increased shoot number, decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and increased oxidative stress tolerance as compared to a control plant. The method steps comprise:
introducing a mutation into the 5′-UTR of a GGP gene in the plant cell or plant, wherein the 5′UTR comprises at least one of:
(i) a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID NOs: 41-100, 111-131, 144-145, 147, 149-150, or 153-155; or
(ii) a sequence encoding a polypeptide with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID NOs: 1-40, 108, 132-137, 146, 148, 151-152, or 156; wherein the mutation disrupts the function of an upstream open reading frame (uORF) encoded by the 5′UTR; and wherein said mutation is at least one of a deletion, an addition, or a substitution of at least one nucleotide in the 5′UTR.

EXAMPLE 34. The method of Example 33 wherein the mutation is in the uORF sequence in the 5′UTR.

EXAMPLE 35. The method of Example 33 wherein the uORF has a sequence selected from any one of SEQ ID NO: 41 to 60 and 129 to 131 or a variant thereof with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID NO: 41 to 60 and 129 and 131.

### Example 36. A Method for Selecting a Plant With One or More Desirable Traits

In this example, the one or more desirable traits are selected from the group consisting of: increased ascorbate level, increased shoot number, increased biomass, decreased free radical damage, decreased free radical level, increased stress tolerance, and increased oxidative stress tolerance; as compared to a control plant. The method steps comprise:
selecting a plant for the presence of a first polymorphism in a polynucleotide comprising a sequence encoding a polypeptide with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to an amino acid sequence selected from SEQ ID NO:1 to 20, and 132 to 134 in the plant, or a further polymorphism linked to the first polymorphism;
wherein the first polymorphism disrupts expression of the polypeptide, thus producing the selected plant with the one or more desirable traits.

EXAMPLE 37. The method of Example 36, wherein the selected plant is further selected for increased biomass, increased shoot number, decreased free radical damage, decreased free radical level, increased stress tolerance, increased abiotic stress tolerance, increased biotic stress tolerance, increased disease tolerance, increased discase resistance, increased nutrient use efficiency, increased nitrogen use efficiency, or increased oxidative stress tolerance as compared to the control plant.

EXAMPLE 38. The method of Example 36, wherein the method includes the step of separating the selected plant from one or more of non-selected plants.

TABLE 9
SUMMARY OF SEQUENCES

| | Molecule | | | |
|---|---|---|---|---|
| SEQ ID NO. | type | Species | Classification | Reference |
| 1 | polypeptide | Actinidia eriantha | Dicot | uORF peptide |
| 2 | polypeptide | Cucumis sativus | Dicot | uORF peptide |
| 3 | polypeptide | Glycine max | Dicot | uORF peptide |
| 4 | polypeptide | Solanum lycopersicum | Dicot | uORF peptide |
| 5 | polypeptide | Vitis vinifera | Dicot | uORF peptide |
| 6 | polypeptide | Arabidopsis thaliana | Dicot | uORF peptide |
| 7 | polypeptide | Arabidopsis thaliana | Dicot | uORF peptide at5g55120 |
| 8 | polypeptide | Malus × domesticus | Dicot | uORF peptide |
| 9 | polypeptide | Medicago truncatula | Dicot | uORF peptide |
| 10 | polypeptide | Populus trichocarpa | Dicot | uORF peptide |
| 11 | polypeptide | Picea sitchensis | Pinophyta | uORF peptide |
| 12 | polypeptide | Physcomitrella patens | Bryophyta | uORF peptide |
| 13 | polypeptide | Actinidia arguta | Dicot | uORF peptide |
| 14 | polypeptide | Actinidia eriantha | Dicot | uORF peptide |
| 15 | polypeptide | Actinidia chinensis | Dicot | uORF peptide |
| 16 | polypeptide | Fragaria vulgaris | Dicot | uORF peptide |
| 17 | polypeptide | Solanum tuberosum | Dicot | uORF peptide |
| 18 | polypeptide | Chlamydomonas | Protista | uORF peptide |
| | | reinhardtii | | |
| 19 | polypeptide | Zea mays | Monocot | uORF peptide |
| 20 | polypeptide | Selaginella | Lycopodiophyta | uORF peptide |
| | | moellendorffii | | |
| 21 | polypeptide | Actinidia eriantha | Dicot | uORF peptide conserved region |
| 22 | polypeptide | Cucumis sativus | Dicot | uORF peptide conserved region 2 |
| 23 | polypeptide | Glycine max | Dicot | uORF peptide conserved region |
| 24 | polypeptide | Solanum lycopersicum | Dicot | uORF peptide conserved region |
| 25 | polypeptide | Vitis vinifera | Dicot | uORF peptide conserved region |
| 26 | polypeptide | Arabidopsis thaliana | Dicot | uORF peptide conserved region |
| | | | | at4g26850 |
| 27 | polypeptide | Arabidopsis thaliana | Dicot | uORF peptide conserved region |
| | | | | at5g55120 |
| 28 | polypeptide | Malus × domesticus | Dicot | uORF peptide conserved region |
| 29 | polypeptide | Medicago truncatula | Dicot | uORF peptide conserved region |
| 30 | polypeptide | Populus trichocarpa | Dicot | uORF peptide conserved region |
| 31 | polypeptide | Picea sitchensis | Pinophyta | uORF peptide conserved region |
| 32 | polypeptide | Physcomitrella | Bryophyta | uORF peptide patens conserved |
| | | | | region |
| 33 | polypeptide | Actinidia arguta | Dicot | uORF peptide conserved region |
| 34 | polypeptide | Actinidia eriantha | Dicot | uORF peptide conserved region |
| 35 | polypeptide | Actinidia chinensis | Dicot | uORF peptide conserved region |
| 36 | polypeptide | Fragaria vulgaris | Dicot | uORF peptide conserved region |
| 37 | polypeptide | Solanum tuberosum | Dicot | uORF peptide conserved region |
| 38 | polypeptide | Chlamydomonas | Protista | uORF peptide conserved region |
| | | reinhardtii | | |
| 39 | polypeptide | Zea mays | Monocot | uORF peptide conserved region |
| 40 | polypeptide | Selaginella | Lycopodiophyta | uORF peptide conserved region |
| | | moellendorffii | | |
| 41 | polynucleotide | Actinidia eriantha | Dicot | uORF DNA |
| 42 | polynucleotide | Cucumis sativus | Dicot | uORF DNA |
| 43 | polynucleotide | Glycine max | Dicot | uORF DNA |
| 44 | polynucleotide | Solanum lycopersicum | Dicot | uORF DNA |
| 45 | polynucleotide | Vitis vinifera | Dicot | uORF DNA |
| 46 | polynucleotide | Arabidopsis thaliana | Dicot | uORF DNA at4g26850 |
| 47 | polynucleotide | Arabidopsis thaliana | Dicot | uORF DNA at5g55120 |
| 48 | polynucleotide | Malus × domesticus | Dicot | uORF DNA |
| 49 | polynucleotide | Medicago truncatula | Dicot | uORF DNA |
| 50 | polynucleotide | Populus trichocarpa | Dicot | uORF DNA |
| 51 | polynucleotide | Picea sitchensis | Pinophyta | uORF DNA |
| 52 | polynucleotide | Physcomitrella patens | Bryophyta | uORF DNA |
| 53 | polynucleotide | Actinidia arguta | Dicot | uORF DNA |
| 54 | polynucleotide | Actinidia eriantha | Dicot | uORF DNA |
| 55 | polynucleotide | Actinidia chinensis | Dicot | uORF DNA |
| 56 | polynucleotide | Fragaria vulgaris | Dicot | uORF DNA |
| 57 | polynucleotide | Solanum tuberosum | Dicot | uORF DNA |
| 58 | polynucleotide | Chlamydomonas | Protista | uORF DNA |
| | | reinhardtii | | |
| 59 | polynucleotide | Zea mays | Monocot | uORF DNA |
| 60 | polynucleotide | Selaginella | Lycopodiaphyta | uORF DNA |
| | | moellendorffii | | |
| 61 | polynucleotide | Actinidia eriantha | Dicot | uORF DNA conserved region |
| 62 | polynucleotide | Cucumis sativus | Dicot | uORF DNA conserved region |
| 63 | polynucleotide | Glycine max | Dicot | uORF DNA conserved region |
| 64 | polynucleotide | Solanum lycopersicum | Dicot | uORF DNA conserved region |
| 65 | polynucleotide | Vitis vinifera | Dicot | uORF DNA conserved region |
| 66 | polynucleotide | Arabidopsis thaliana | Dicot | uORF DNA conserved region |
| | | | | at4g26850 |
| 67 | polynucleotide | Arabidopsis thaliana | Dicot | uORF DNA conserved region |
| | | | | at5g55120 |
| 68 | polynucleotide | Malus × domesticus | Dicot | uORF DNA conserved region |
| 69 | polynucleotide | Medicago truncatula | Dicot | uORF DNA conserved region |
| 70 | polynucleotide | Populus trichocarpa | Dicot | uORF DNA conserved region |
| 71 | polynucleotide | Picea sitchensis | Pinophyta | uORF DNA conserved region |
| 72 | polynucleotide | Physcomitrella | Bryophyta | uORF DNA patens conserved |
| | | | | region |
| 73 | polynucleotide | Actinidia arguta | Dicot | uORF DNA conserved region |
| 74 | polynucleotide | Actinidia eriantha | Dicot | uORF DNA conserved region |
| 75 | polynucleotide | Actinidia chinensis | Dicot | uORF DNA conserved region 7 |
| 76 | polynucleotide | Fragaria vulgaris | Dicot | uORF DNA conserved region |
| 77 | polynucleotide | Solanum | Dicot | uORF DNA tuberosum conserved |
| | | | | region |
| 78 | polynucleotide | Chlamydomonas | Protista | uORF DNA reinhardtii conserved |
| | | | | region |
| 79 | polynucleotide | Zea mays | Monocot | uORF DNA conserved region |
| 80 | polynucleotide | Selaginella | Lycopodiophyta | uORF DNA conserved region |
| | | moellendorffii | | |
| 81 | polynucleotide | Actinidia eriantha | Dicot | Whole 5′-UTR |
| 82 | polynucleotide | Cucumis sativus | Dicot | Whole 5′-UTR |
| 83 | polynucleotide | Glycine max | Dicot | Whole 5′-UTR |
| 84 | polynucleotide | Solanum lycopersicum | Dicot | Whole 5′-UTR |
| 85 | polynucleotide | Vitis vinifera | Dicot | Whole 5′-UTR |
| 86 | polynucleotide | Arabidopsis thaliana | Dicot | Whole 5′-UTR at4g26850 |
| 87 | polynucleotide | Arabidopsis thaliana | Dicot | Whole 5′-UTR at5g55120 |
| 88 | polynucleotide | Malus × domesticus | Dicot | Whole 5′-UTR |
| 89 | polynucleotide | Medicago truncatula | Dicot | Whole 5′-UTR |
| 90 | polynucleotide | Populus trichocarpa | Dicot | Whole 5′-UTR |
| 91 | polynucleotide | Picea sitchensis | Pinophyta | Whole 5′-UTR |
| 92 | polynucleotide | Physcomitrella patens | Bryophyta | Whole 5′-UTR |
| 93 | polynucleotide | Actinidia arguta | Dicot | Whole 5′-UTR |
| 94 | polynucleotide | Actinidia eriantha | Dicot | Whole 5′-UTR |
| 95 | polynucleotide | Actinidia chinensis | Dicot | Whole 5′-UTR |
| 96 | polynucleotide | Fragaria vulgaris | Dicot | Whole 5′-UTR |
| 97 | polynucleotide | Solanum tuberosum | Dicot | Whole 5′-UTR |
| 98 | polynucleotide | Chlamydomonas | Protista | Whole 5′-UTR |
| | | reinhardtii | | |
| 99 | polynucleotide | Zea mays | Monocot | Whole 5′-UTR |
| 100 | polynucleotide | Selaginella | Lycopodiophyta | Whole 5′-UTR |
| | | moellendorffii | | |
| 101 | polynucleotide | Actinidia eriantha | Dicot | GGP genomic promoter sequence |
| 102 | polynucleotide | Arabidopsis thaliana | Dicot | GGP genomic promoter sequence |
| | | | | at4g26850 |
| 103 | polynucleotide | Arabidopsis thaliana | Dicot | GGP genomic promoter sequence |
| | | | | at5g55120 |
| 104 | polynucleotide | Malus × domesticus | Dicot | GGP2 genomic promoter sequence |
| | | | | MDP0000288088 |
| 105 | polynucleotide | Malus × domesticus | Dicot | GGP1 genomic promoter sequence |
| | | | | MDP0000172222 |
| 106 | polynucleotide | Malus × domesticus | Dicot | GGP3 genomic promoter sequence |
| | | | | MDP0000191488 |
| 107 | polynucleotide | Actinidia chinensis | Dicot | GGP genomic promoter sequence |
| 108 | polypeptide | Artificial | Consensus | Consensus motif |
| 109 | polynucleotide | Artificial | Primer | LUC1 |
| 110 | polynucleotide | Artificial | Primer | LUC2 |
| 111 | polynucleotide | Cucumis sativus | Dicot | Sub-sequence of 5′-UTR |
| 112 | polynucleotide | Actinidia eriantha | Dicot | Sub-sequence of 5′-UTR |
| 113 | polynucleotide | Actinidia arguta | Dicot | Sub-sequence of 5′-UTR |
| 114 | polynucleotide | Actinidia eriantha | Dicot | Sub-sequence of 5′-UTR |
| 115 | polynucleotide | Actinidia chinensis | Dicot | Sub-sequence of 5′-UTR |
| 116 | polynucleotide | Malus × domesticus | Dicot | Sub-sequence of 5′-UTR |
| 117 | polynucleotide | Fragaria vulgaris | Dicot | Sub-sequence of 5′-UTR |
| 118 | polynucleotide | Arabidopsis thaliana | Dicot | Sub-sequence of 5′-UTR |
| 119 | polynucleotide | Arabidopsis thaliana | Dicot | Sub-sequence of 5′-UTR |
| 120 | polynucleotide | Populus trichocarpa | Dicot | Sub-sequence of 5′-UTR |
| 121 | polynucleotide | Glycine max | Dicot | Sub-sequence of 5′-UTR |
| 122 | polynucleotide | Medicago truncatula | Dicot | Sub-sequence of 5′-UTR |
| 123 | polynucleotide | Solanum lycopersicum | Dicot | Sub-sequence of 5′-UTR |
| 124 | polynucleotide | Solanum tuberosum | Dicot | Sub-sequence of 5′-UTR |
| 125 | polynucleotide | Vitis vinifera | Dicot | Sub-sequence of 5′-UTR |
| 126 | polynucleotide | Solanum lycopersicum | Dicot | Whole 5′-UTR |
| 127 | polynucleotide | Solanum tuberosum | Dicot | Whole 5′-UTR |
| 128 | polynucleotide | Malus × domesticus | Dicot | Whole 5′-UTR |
| 129 | polynucleotide | Solanum lycopersicum | Dicot | uORF DNA |
| 130 | polynucleotide | Solanum tuberosum | Dicot | uORF DNA |
| 131 | polynucleotide | Malus × domesticus | Dicot | uORF DNA |
| 132 | polypeptide | Solanum lycopersicum | Dicot | uORF peptide |
| 133 | polypeptide | Solanum tuberosum | Dicot | uORF peptide |
| 134 | polypeptide | Malus × domesticus | Dicot | uORF peptide |
| 135 | polypeptide | Solanum lycopersicum | Dicot | uORF peptide conserved region |
| 136 | polypeptide | Solanum tuberosum | Dicot | uORF peptide conserved region |
| 137 | polypeptide | Malus × domesticus | Dicot | uORF peptide conserved region |
| 138 | polynucleotide | Solanum lycopersicum | Dicot | uORF DNA conserved region |
| 139 | polynucleotide | Solanum lycopersicum | Dicot | uORF DNA conserved region |
| 140 | polynucleotide | Malus × domesticus | Dicot | uORF DNA conserved region |
| 141 | polynucleotide | Artificial | Consensus | Consensus sequence |
| 142 | polypeptide | Artificial | Consensus | Consensus sequence |
| 143 | polynucleotide | Artificial | Construct | Artificial construct |
| 144 | polynucleotide | Arabidopsis thaliana | Dicot | GGP2 sequence AT4G26850(VTC2) |
| 145 | polynucleotide | Arabidopsis thaliana | Dicot | KCW66953 cdna protein coding |
| 146 | polypeptide | Arabidopsis thaliana | Dicot | KCW66953 peptide KCW66953 |
| | | | | pep: proteincoding |
| 147 | polynucleotide | Arabidopsis thaliana | Dicot | KCW66953 utr5 |
| 148 | polypeptide | Arabidopsis thaliana | Dicot | uORF1 |
| 149 | polynucleotide | Miscanthus sinensis | Monocot | Chr15: 21403555 . . . 21407164 |
| | | | | (+strand) class = mRNA |
| | | | | length = 3610 |
| 150 | polynucleotide | Miscanthus sinensis | Monocot | Chr15: 21403555 . . . 21404063 |
| | | | | (+strand) class = five_prime |
| | | | | UTR length = 509 |
| 151 | polypeptide | Miscanthus sinensis | Monocot | v7.1\|Misin15G081100.1.p |
| 152 | polypeptide | Miscanthus sinensis | Monocot | 1cl\|ORF2 |
| 153 | polynucleotide | Saccharum officinarum | Monocot | Sh_249J15 Sh_249J15: 5122 . . . 8722 |
| | | | | (−strand) class = mRNA length = 3601 |
| 154 | polynucleotide | Medicago truncatula | Dicot | AET00558 cdna: protein coding |
| 155 | polynucleotide | Medicago truncatula | Dicot | AET00558 cds: protein coding |
| 156 | polypeptide | Medicago truncatula | Dicot | AET00558 peptide: AET00558 |
| | | | | pep: protein coding |
| 157 | polypeptide | Arabidopsis thaliana | Dicot | Protein product of Arabidopsis locus |
| | | | | AT4G26850 |
| 158 | polypeptide | Arabidopsis thaliana | Dicot | Protein product of Arabidopsis locus |
| | | | | AT5G55120.1, VTC5 |
| 159 | polypeptide | Solanum lycopersicum | Dicot | GDP-L-galactose phosphorylase |
| | | | | NCBI Ref Seq: NP_001266145.1 |
| 160 | polypeptide | Solanum lycopersicum | Dicot | GDP-L-galactose phosphorylase |
| | | | | GenBank: AFD54988.1 |
| 161 | polypeptide | Solanum lycopersicum | Dicot | GDP-L-galactose phosphorylase |
| | | | | NCBI Ref Seq: NP_001334398.1 |
| 162 | polypeptide | Solanum lycopersicum | Dicot | GDP-L-galactose phosphorylase 1 |
| | | | | GenBank: AZZ09175.1 |
| 163 | polypeptide | Triticum aestivum | Monocot | GDP-L-galactose |
| | | | | phosphorylaseGenBank: |
| | | | | QGQ63671.1 |
| 164 | polypeptide | Triticum aestivum | Monocot | GDP-L-galactose phosphorylase |
| | | | | GenBank: QGQ63670.1 |
| 165 | polypeptide | Triticum aestivum | Monocot | GDP-L-galactose phosphory lase |
| | | | | GenBank: QGQ63669.1 |
| 166 | polypeptide | Triticum aestivum | Monocot | GDP-L-galactose phosphorylase |
| | | | | GenBank: QGQ63668.1 |
| 167 | polypeptide | Triticum aestivum | Monocot | GDP-L-galactose phosphorylase |
| | | | | GenBank: QGQ63667.1 |
| 168 | polypeptide | Triticum aestivum | Monocot | GDP-L-galactose phosphorylase |
| | | | | GenBank: QGQ63666.1 |
| 169 | polypeptide | Triticum aestivum | Monocot | GDP-L-galactose phosphorylase |
| | | | | 2-like NCBI Ref |
| | | | | Seq: XP_044395927.1 |
| 170 | polypeptide | Triticum aestivum | Monocot | GDP-L-galactose phosphorylase |
| | | | | 2-like NCBI Ref |
| | | | | Seq: XP_044387514.1 |
| 171 | polypeptide | Triticum aestivum | Monocot | GDP-L-galactose phosphorylase |
| | | | | 2-like NCBI Ref Seq: |
| | | | | XP_044387513.1 |
| 172 | polypeptide | Triticum aestivum | Monocot | GDP-L-galactose phosphorylase |
| | | | | 2-like NCBI Ref Seq: |
| | | | | XP_044387512.1 |
| 173 | polypeptide | Triticum aestivum | Monocot | GDP-L-galactose phosphorylase |
| | | | | 2-like NCBI Ref Seq: |
| | | | | XP_044387511.1 |
| 174 | polypeptide | Triticum aestivum | Monocot | GDP-L-galactose phosphorylase |
| | | | | 2-like XP_044380127.1 |
| 175 | polypeptide | Triticum aestivum | Monocot | GDP-L-galactose phosphorylase |
| | | | | 2-like NCBI Ref Seq: |
| | | | | XP_044373977.1 |
| 176 | polypeptide | Triticum aestivum | Monocot | GDP-L-galactose phosphorylase |
| | | | | 2-like NCBI Ref Seq: |
| | | | | XP_044368801.1 |
| 177 | polypeptide | Triticum aestivum | Monocot | GDP-L-galactose phosphorylase |
| | | | | 2-like NCBI Ref Seq: |
| | | | | XP_044364227.1 |
| 178 | polypeptide | Triticum aestivum | Monocot | GDP-L-galactose phosphorylase |
| | | | | 1-like NCBI Ref Seq: |
| | | | | XP_044395136.1 |
| 179 | polypeptide | Triticum aestivum | Monocot | GDP-L-galactose phosphorylase |
| | | | | 1-like NCBI Ref Seq: |
| | | | | XP_044395135.1 |
| 180 | polypeptide | Triticum aestivum | Monocot | GDP-L-galactose phosphorylase |
| | | | | 1-like NCBI Ref Seq: |
| | | | | XP_044386803.1 |
| 181 | polypeptide | Triticum aestivum | Monocot | GDP-L-galactose phosphorylase |
| | | | | 1-like NCBI Ref Seq: |
| | | | | XP_044386802.1 |
| 182 | polypeptide | Triticum aestivum | Monocot | GDP-L-galactose phosphorylase |
| | | | | 1-like NCBI Ref Seq: |
| | | | | XP_044379447.1 |
| 183 | polypeptide | Triticum aestivum | Monocot | GDP-L-galactose phosphorylase |
| | | | | 1-like NCBI Ref Seq: |
| | | | | XP_044379445.1 |
| 184 | polypeptide | Triticum aestivum | Monocot | GDP-L-galactose phosphorylase |
| | | | | 1-like NCBI Ref Seq: |
| | | | | XP_044379444.1 |
| 185 | polypeptide | Triticum aestivum | Monocot | GDP-L-galactose phosphorylase |
| | | | | 1-like isoform X7 NCBI Ref Seq: |
| | | | | XP_044351196.1 |
| 186 | polypeptide | Triticum aestivum | Monocot | GDP-L-galactose phosphorylase 1- |
| | | | | like isoform X7 NCBI Ref Seq: |
| | | | | XP_044351195.1 |
| 187 | polypeptide | Triticum aestivum | Monocot | GDP-L-galactose phosphorylase 1- |
| | | | | like isoform X7 NCBI Ref Seq: |
| | | | | XP_044351194.1 |
| 188 | polypeptide | Triticum aestivum | Monocot | GDP-L-galactose phosphorylase 1- |
| | | | | like isoform X6 NCBI Ref Seq: |
| | | | | XP_044351193.1 |
| 189 | polypeptide | Triticum aestivum | Monocot | GDP-L-galactose phosphorylase 1- |
| | | | | like isoform X6 NCBI Ref Seq: |
| | | | | XP_044351192.1 |
| 190 | polypeptide | Triticum aestivum | Monocot | GDP-L-galactose phosphorylase 1- |
| | | | | like isoform X5 NCBI Ref Seq: |
| | | | | XP_044351191.1 |
| 191 | polypeptide | Triticum aestivum | Monocot | GDP-L-galactose phosphorylase 1- |
| | | | | like isoform X5 NCBI Ref Seq: |
| | | | | XP_044351190.1 |
| 192 | polypeptide | Triticum aestivum | Monocot | GDP-L-galactose phosphorylase 1- |
| | | | | like isoform X5 NCBI Ref Seq: |
| | | | | XP_044351189.1 |
| 193 | polypeptide | Triticum aestivum | Monocot | GDP-L-galactose phosphorylase 1- |
| | | | | like isoform X4 NCBI Ref Seq: |
| | | | | XP_044351188.1 |
| 194 | polypeptide | Triticum aestivum | Monocot | GDP-L-galactose phosphorylase 1- |
| | | | | like isoform X4 NCBI Ref Seq: |
| | | | | XP_044351187.1 |
| 195 | polypeptide | Triticum aestivum | Monocot | GDP-L-galactose phosphorylase 1- |
| | | | | like isoform X3 NCBI Ref Seq: |
| | | | | XP_044351186.1 |
| 196 | polypeptide | Triticum aestivum | Monocot | GDP-L-galactose phosphorylase 1- |
| | | | | like isoform X2 NCBI Ref Seq: |
| | | | | XP_044351185.1 |
| 197 | polypeptide | Triticum aestivum | Monocot | GDP-L-galactose phosphorylase 1- |
| | | | | like isoform X1 NCBI Ref Seq: |
| | | | | XP_044351184.1 |
| 198 | polypeptide | Triticum aestivum | Monocot | GDP-L-galactose phosphorylase 1- |
| | | | | like isoform X3 NCBI Ref Seq: |
| | | | | XP_044342439.1 |
| 199 | polypeptide | Triticum aestivum | Monocot | GDP-L-galactose phosphorylase 1- |
| | | | | like isoform X2 NCBI Ref Seq: |
| | | | | XP_044342438.1 |
| 200 | polypeptide | Triticum aestivum | Monocot | GDP-L-galactose phosphorylase 1- |
| | | | | like isoform X1 NCBI Ref Seq: |
| | | | | XP_044342437.1 |
| 201 | polypeptide | Glycine max | Dicot | GDP-L-galactose |
| | | | | phosphorylaseGenBank: |
| | | | | AEO21431.1 |
| 202 | polypeptide | Glycine max | Dicot | GDP-L-galactose phosphorylase 1- |
| | | | | like NCBI Ref Seq: |
| | | | | NP_001240984.1 |
| 203 | polypeptide | Glycine max | Dicot | GDP-L-galactose phosphorylase 1 |
| | | | | GenBank: KAH1263738.1 |
| 204 | polypeptide | Glycine max | Dicot | GDP-L-galactose phosphorylase 1 |
| | | | | GenBank: KAH1239561.1 |
| 205 | polypeptide | Glycine max | Dicot | GDP-L-galactose phosphorylase 1 |
| | | | | GenBank: KAH1211432.1 |
| 206 | polypeptide | Glycine max | Dicot | GDP-L-galactose phosphorylase |
| | | | | 1GenBank: KAH1197362.1 |
| 207 | polypeptide | Glycine max | Dicot | GDP-L-galactose phosphorylase 1 |
| | | | | NCBI Ref Seq: XP_003519588.1 |
| 208 | polypeptide | Glycine max | Dicot | GDP-L-galactose phosphorylase |
| | | | | NCBI Ref Seq: XP_003551872.1 |
| 209 | polypeptide | Solanum tuberosum | Dicot | GDP-L-galactose phosphorylase 1- |
| | | | | like NCBI Ref Seq: |
| | | | | NP_001275193.1 |
| 210 | polypeptide | Solanum tuberosum | Dicot | GDP-L-galactose phosphorylase 1- |
| | | | | like NCBI Ref Seq: |
| | | | | NP_001275300.1 |
| 211 | polypeptide | Zea mays | Monocot | GDP-L-galactose phosphorylase 2- |
| | | | | like NCBI Ref Seq: |
| | | | | NP_001140486.1 |
| 212 | polypeptide | Zea mays | Monocot | GDP-L-galactose phosphorylase 2 |
| | | | | GenBank: PWZ16926.1 |
| 213 | polypeptide | Zea mays | Monocot | GDP-L-galactose phosphorylase 1 |
| | | | | GenBank: AQK78696.1 |
| 214 | polypeptide | Zea mays | Monocot | GDP-L-galactose phosphorylase 1 |
| | | | | GenBank: AQK78695.1 |
| 215 | polypeptide | Zea mays | Monocot | GDP-L-galactose phosphorylase 1 |
| | | | | GenBank: AQK78694.1 |
| 216 | polypeptide | Zea mays | Monocot | GDP-L-galactose phosphorylase 2 |
| | | | | GenBank PWZ34063.1 |
| 217 | polypeptide | Zea mays | Monocot | GDP-L-galactose phosphorylase 1 |
| | | | | GenBank: ONM35503.1 |
| 218 | polypeptide | Zea mays | Monocot | GDP-L-galactose phosphorylase 1 |
| | | | | GenBank: ONM35501.1 |
| 219 | polypeptide | Zea mays | Monocot | VTC2NCBI Ref Seq: |
| | | | | NP_001150222.2 VTC2 |
| 220 | polypeptide | Prunus dulcis | Dicot | GDP-L-galactose phosphorylase 2- |
| | | | | like NCBI Ref Seq: |
| | | | | XP_034211435.1 |
| 221 | polypeptide | Prunus dulcis | Dicot | GDP-L-galactose phosphorylase 1- |
| | | | | like NCBI Ref Seq: |
| | | | | XP_034211105.1 |
| 222 | polypeptide | Prunus dulcis | Dicot | hypothetical protein L3X38_023955 |
| | | | | GenBank: KAI5333823.1 |
| 223 | polypeptide | Prunus dulcis | Dicot | hypothetical protein L3X38_021754 |
| | | | | GenBank: KAI5331628.1 |
| 224 | polypeptide | Solanum lycopersicum | Dicot | GDP-L-galactose phosphorylase |
| | | | | NCBI Ref Seq: NP_001266145.1 |
| 225 | polypeptide | Solanum lycopersicum | Dicot | GDP-L-galactose phosphorylase |
| | | | | NCBI Ref Seq: NP_001334398.1 |
| 226 | polypeptide | Oryza sativa | Monocot | GDP-L-galactose phosphorylase 2 |
| | | | | isoform X2 NCBI Ref Seq: |
| | | | | XP_025878193.1 |
| 227 | polypeptide | Oryza sativa | Monocot | GDP-L-galactose phosphorylase 2 |
| | | | | isoform X1 NCBI Ref Seq: |
| | | | | XP_025878192.1 |
| 228 | polypeptide | Oryza sativa | Monocot | GDP-L-galactose phosphorylase 2 |
| | | | | NCBI Ref Seq: XP_015620659.1 |
| 229 | polypeptide | Oryza sativa | Monocot | GDP-L-galactose phosphorylase 2 |
| | | | | NCBI Ref Seq: XP_015619890.1 |
| 230 | polypeptide | Oryza sativa | Monocot | GDP-L-galactose phosphorylase 2 |
| | | | | isoform X2 NCBI Ref Seq: |
| | | | | XP_015629544.1 |
| 231 | polypeptide | Oryza sativa | Monocot | GDP-L-galactose phosphorylase 2 |
| | | | | isoform X2 NCBI Ref Seq: |
| | | | | XP_015629539.1 |
| 232 | polypeptide | Actinidia chinensis | Dicot | transferase SEQ ID NO: 1 in U.S. Pat. No. |
| | | | | 9,157,088B2 from Actinidia chinensis |
| | | | | Ref EST 319998 Ac |
| 233 | polypeptide | Malus domestica | Dicot | transferase SEQ ID NO: 2 in U.S. Pat. No. |
| | | | | 9,157,088B2 from Malus × domesticus |
| | | | | Ref EST 82552 Md |
| 234 | polypeptide | Actinidia chinensis | Dicot | transferase SEQ ID NO: 3 in U.S. Pat. No. |
| | | | | 9,157,088B2 from Actinidia chinensis |
| | | | | Ref EST 244893 Ac |
| 235 | polypeptide | Actinidia eriantha | Dicot | transferase SEQ ID NO: 4 in U.S. Pat. No. |
| | | | | 9,157,088B2 from Actinidia chinensis |
| | | | | Ref EST 24547 Ae |
| 236 | polypeptide | Actinidia eriantha | Dicot | transferase SEQ ID NO: 5 in U.S. Pat. No. |
| | | | | 9,157,088B2 from Actinidia chinensis |
| | | | | Ref EST 276582 Ae |
| 237 | polypeptide | Malus sieboldii | Dicot | transferase SEQ ID NO: 6 in U.S. Pat. No. |
| | | | | 9,157,088B2 from Malus sieboldii |
| | | | | Ref EST 315905 Ms |
| 238 | polypeptide | Solanum tuberosum | Dicot | transferase SEQ ID NO: 7 in U.S. Pat. No. |
| | | | | 9,157,088B2 from Solanum |
| | | | | tuberosum Ref contig st |
| 239 | polypeptide | Arabidopsis thaliana | Dicot | transferase SEQ ID NO: 8 in U.S. Pat. No. |
| | | | | 9,157,088B2 from Arabidopsis |
| | | | | thaliana Ref At4g26850 |
| 240 | polypeptide | Arabidopsis thaliana | Dicot | transferase SEQ ID NO: 9 in U.S. Pat. No. |
| | | | | 9,157,088B2 from Arabidopsis |
| | | | | thaliana Ref At5g55120 |
| 241 | polypeptide | Solanum lycopersicum | Dicot | transferase SEQ ID NO: 10 in U.S. Pat. No. |
| | | | | 9,157,088B2 from Solanum |
| | | | | lycopersicum Ref BTO13858 Le |
| 242 | polypeptide | Oryza sativa | Monocot | transferase SEQ ID NO: 11 in U.S. Pat. No. |
| | | | | 9,157,088B2 from Oryza sativa |
| | | | | Ref Os12g0190000 |

“Ref Seq” refers to “reference sequence”

What is claimed is:

1. A selected transformed crop plant that comprises a polynucleotide sequence comprising a constitutive, tissue-specific, or inducible promoter fused to DNA coding sequence, wherein the DNA coding sequence encodes a polypeptide that is a Homolog of the Arabidopsis GGP protein or that is at least 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identical to any of SEQ ID NO: 157-242; and wherein the transformed plant has been selected for a desirable trait; and
wherein the selected transformed crop plant exhibits a desirable trait selected from the group consisting of increased ascorbate level, increased biomass, increased shoot number, decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and increased oxidative stress tolerance.

2. The selected transformed crop plant of claim 1, wherein the transformed plant has been selected for the desirable trait from a pool of plants that comprise the polynucleotide sequence.

3. The selected transformed crop plant of claim 1, wherein the crop is tomato or a member of the Solanaceae family.

4. The selected transformed crop of crop of claim 1 wherein the crop is a non-food crop.

5. The selected transformed crop plant of claim 1, wherein the crop is Miscanthus or another perennial grass.

6. The selected transformed crop plant of claim 1, wherein the crop is Eucalyptus, almond, or another tree species.

7. The selected transformed crop plant of claim 1, wherein the promoter sequence has at least 70% identity to a promoter sequence selected from the following list: 35S, rice actin, corn tubulin, RD29A, RD29B, a ubiquitin promoter, a drought inducible promoter (including those drought inducible promoters disclosed in U.S. Pat. No. 8,895,305) or a disease-inducible promoter.

8. The selected transformed crop plant of claim 1, wherein selected transformed crop plant is confirmed to exhibit the desirable trait in the absence of other undesirable traits such as organ abnormalities and/or stunted growth.

9. A selected plant or plant part harboring a non-native allele of a gene of interest that controls a desirable trait wherein the allele comprises a selected sequence change in a uORF within the 5′UTR which results in an increase in the level of the polypeptide produced by the gene of interest compared to the level of a polypeptide in a control plant that lacks the allele grown under comparable conditions, and wherein the polypeptide is an Homolog of the Arabidopsis GGP protein or is a polypeptide that is at least 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identical to any of SEQ ID NO: 157-242; and wherein the plant exhibits a desirable trait selected from the group consisting of increased ascorbate level, increased biomass, increased shoot number, decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and increased oxidative stress tolerance.

10. The selected plant or plant part of claim 9, wherein the plant harboring the non-native allele has been selected for the desirable trait from a pool of plants that harbor sequence changes in the uORF.

11. The selected plant or plant part of claim 9, wherein the plant is tomato or a member of the Solanaceae family.

12. The selected plant or plant part of claim 9, wherein the crop is a non-food crop.

13. The selected plant or plant part of claim 9, wherein the crop is Miscanthus or another perennial grass.

14. The selected plant or plant part of claim 9, wherein the crop is Eucalyptus, almond, or another tree species.

15. The selected plant or plant part of claim 9, wherein selected plant is confirmed to exhibit the desirable trait in the absence of other undesirable traits such as organ abnormalities and/or stunted growth.

16. A method for upregulating a desirable trait in a plant, the method comprising:
a. providing a nucleic acid comprising a 5′ UTR (untranslated region) obtained from a region upstream of a gene of interest that directly or indirectly upregulates the desirable trait, wherein the 5′ UTR comprises an upstream open reading frame (uORF);
wherein the 5′UTR comprises at least one of:
iii) a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID Nos: 41-100, 111-131, 144-145, 147, 149-150, or 153-1551; or
iv) a sequence encoding a polypeptide with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or 100% identity to any one of SEQ ID NOs: 1-40, 108, 132-137, 146, 148, 151-152, or 156;
v) wherein the 5′UTR has a mutation introduced that that putatively disrupts the function of an upstream open reading frame (uORF) encoded by the 5′UTR; and wherein said mutation is at least one of a deletion, an addition, or a substitution of at least one nucleotide in the 5′UTR;
b. fusing the nucleic acid to a reporter gene to create a 5′ UTR::reporter polynucleotide;
c. introducing a mutation into said nucleic acid to create a 5′ UTR::reporter polynucleotide with the mutation in the uORF;
d. introducing the 5′ UTR::reporter polynucleotide into a plant cell;
e. measuring a level of a product from the reporter gene in the plant cell;
f. identifying a mutation that produces less of the product in the plant cell than the level of the product in a control plant cell comprising a control 5′ UTR::reporter polynucleotide in which the uORF has been deleted from the 5′ UTR fused to the reporter gene; and wherein
g. the identified uORF mutation from the reporter analysis, or a comparable mutation, is introduced by gene editing or selection into the uORF at its endogenous locus in the genome of a target plant or target plant cell which is then selected from a population of plants or plants or plant cells and confirmed for the presence of the upregulated desirable trait.

17. The method of claim 16, wherein the level of the product in the plant cell is about 10%, about 20%, about 30%, about 40%, about 50%, about 60%, about 70%, about 80%, or about 90%, or less than 100% of the level of the reporter product produced in the control plant.

18. The method of claim 16, wherein the desirable trait is selected from the group consisting of increased ascorbate level, increased biomass, increased shoot number, decreased free radical damage, decreased free radical level, increased stress tolerance, increased abiotic stress tolerance, increased biotic stress tolerance, increased disease tolerance, increased disease resistance, increased nutrient use efficiency, increased nitrogen use efficiency, and increased oxidative stress tolerance.

19. The method of claim 16, wherein the selected plant or plant cell is confirmed to exhibit the upregulated desirable trait in the absence of other undesirable traits such as organ abnormalities.

20. The method of claim 16, wherein the selected plant or plant cell is or from tomato.
